(19) United States
(12) Patent Application Publication
(10) Pub. No.: US 2022/0130501 A1
(43) Pub. Date: Apr. 28, 2022

(54) CLINICAL DRUG TRIAL DATA ENRICHING USING ACTIVITY AND BEHAVIORAL ANALYTICS CAPTURED WITH PERSONAL DEVICES AND APPS

(71) Applicant: WINKK, INC, Menlo Park, CA (US)

(72) Inventors: Robert O. Keith, Jr., San Jose, CA (US); Sanju Manjunath, San Jose, CA (US)

(21) Appl. No.: 17/573,259

(22) Filed: Jan. 11, 2022

Related U.S. Application Data
(63) Continuation-in-part of application No. 16/868,080, filed on May 6, 2020.
Application No. 16/868,080 is a continuation-in-part of application No. 16/709,683, filed on Dec. 10, 2019.

(51) Int. Cl. G16H 10/20; G16H 40/63; G16H 50/20

(52) U.S. Cl. CPC G16H 10/20; G16H 50/20; G16H 40/63

(57) ABSTRACT

A security platform architecture is described herein. A user identity platform architecture which uses a multitude of biometric analytics to create an identity token unique to an individual human. This token is derived on biometric factors like human behaviors, motion analytics, human physical characteristics like facial patterns, voice recognition prints, usage of device patterns, user location actions and other human behaviors which can derive a token or be used as a dynamic password identifying the unique individual with high calculated confidence. Because of the dynamic nature and the many different factors, this method is extremely difficult to spoof or hack by malicious actors or malware software.

### CROSS-REFERENCE TO RELATED APPLICATION(S)

This application is a continuation-in-part application of co-pending U.S. patent application Ser. No. 16/868,080, filed on May 6, 2020, and titled “USER IDENTIFICATION PROOFING USING A COMBINATION OF USER RESPONSES TO SYSTEM TURING TESTS USING BIOMETRIC METHODS,” which is a continuation-in-part application of co-pending U.S. patent application Ser. No. 16/709,683, filed on Dec. 10, 2019, and titled “SECURITY PLATFORM ARCHITECTURE,” which is hereby incorporated by reference in its entirety for all purposes.

### FIELD OF THE INVENTION

The present invention relates to security. More specifically, the present invention relates to a security architecture.

### BACKGROUND OF THE INVENTION

Although the Internet provides a massive opportunity for shared knowledge, it also enables those with malicious intentions to attack such as by stealing personal data or causing interference with properly functioning mechanisms. The Internet and other networks will continue to grow both in size and functionality, and with such growth, security will be paramount.

### SUMMARY OF THE INVENTION

A security platform architecture is described herein. A user identity platform architecture which uses a multitude of biometric analytics to create an identity token unique to an individual human. This token is derived on biometric factors like human behaviors, motion analytics, human physical characteristics like facial patterns, voice recognition prints, usage of device patterns, user location actions and other human behaviors which can derive a token or be used as a dynamic password identifying the unique individual with high calculated confidence. Because of the dynamic nature and the many different factors, this method is extremely difficult to spoof or hack by malicious actors or malware software.

### BRIEF DESCRIPTION OF THE DRAWINGS

FIG. 1 illustrates a diagram of a security platform architecture according to some embodiments.

FIG. 2 illustrates an exemplary access-hardened API according to some embodiments.

FIG. 3 illustrates a diagram of a secure application architecture according to some embodiments.

FIG. 4 illustrates a diagram of a smart device and a CyberEye multi-factor authentication according to some embodiments.

FIG. 5 illustrates a flowchart of a method of implementing a security platform architecture according to some embodiments.

FIG. 6 illustrates a block diagram of an exemplary computing device configured to implement the security platform architecture according to some embodiments.

FIG. 7 illustrates a diagram of a secure application framework and platform according to some embodiments.

FIG. 8 illustrates a diagram of a secure key exchange through an opti-encryption channel according to some embodiments.

FIG. 9 illustrates a flowchart of a method of utilizing a user device as identification according to some embodiments.

FIG. 10 illustrates a diagram of an optical encryption implementation according to some embodiments.

FIG. 11 illustrates a diagram of an optical encryption implementation on multiple devices according to some embodiments.

FIG. 12 illustrates a diagram of an optical encryption implementation on multiple devices according to some embodiments.

FIG. 13 illustrates a diagram of multiple embedded electronic devices and/or other devices according to some embodiments.

FIG. 14 illustrates a diagram of a system for electronic transactions using personal computing devices and proxy services according to some embodiments.

FIG. 15 illustrates a flowchart of a method of device hand off identification proofing using behavioral analytics according to some embodiments.

FIG. 16 illustrates a flowchart of a method of an automated transparent login without saved credentials or passwords according to some embodiments.

FIG. 17 illustrates a diagram of a system configured for implementing a method of an automated transparent login without saved credentials or passwords according to some embodiments.

FIG. 18 illustrates a flowchart of a method of implementing automated identification proofing using a random multitude of real-time behavioral biometric samplings according to some embodiments.

FIG. 19 illustrates a flowchart of a method of implementing user identification proofing using a combination of user responses to system Turing tests using biometric methods according to some embodiments.

FIG. 20 illustrates a diagram of an aggregated trust framework according to some embodiments.

FIG. 21 illustrates a diagram of mobile trust framework functions according to some embodiments.

FIG. 22 illustrates a diagram of a weighted analytics graph according to some embodiments.

FIG. 23 illustrates diagrams of exemplary scenarios according to some embodiments.

FIG. 24 illustrates a representative diagram of an aggregated trust system including a bus according to some embodiments.

FIG. 25 illustrates a flowchart of a method of using the user as a password according to some embodiments.

FIG. 26 illustrates a diagram of an architectural overview of the ID trust library according to some embodiments.

FIG. 27 illustrates a selection of modules chosen for a given policy according to some embodiments.

FIG. 28 illustrates the logical flow according to some embodiments.

FIG. 29 illustrates a diagram of analytics with shared traits according to some embodiments.

FIG. 30 illustrates a flowchart of a method of implementing analytics with shared traits according to some embodiments.

FIG. 31 illustrates a diagram of a user shaking a user device according to some embodiments.

FIG. 32 illustrates a flowchart of a method of implementing a shake challenge according to some embodiments.

FIG. 33 illustrates a flowchart of a method of implementing device behavior analytics according to some embodiments.

FIG. 34 illustrates a diagram of a device implementing behavior analytics according to some embodiments.

FIG. 35 illustrates a flowchart of a method of utilizing homomorphic encryption according to some embodiments.

FIG. 36 illustrates a flowchart of a method of implementing user identification using voice analytics according to some embodiments.

FIG. 37 illustrates a flowchart of a method of using a multitude of human activities for user identity according to some embodiments.

FIG. 38 illustrates a diagram of an exemplary motion and condition data structure according to some embodiments.

FIG. 39 illustrates a flowchart of a method of implementing a roaming user password based on human identity analytic data according to some embodiments.

FIG. 40 illustrates a diagram of a system implementing a roaming user password based on human identity analytic data according to some embodiments.

FIG. 41 illustrates a flowchart of a method of implementing document signing with the human as the password according to some embodiments.

FIG. 42 illustrates a diagram of a system for document signing with digital signatures with the human as the password.

FIG. 43 illustrates a flowchart of a method of implementing breath pattern analytics according to some embodiments.

FIG. 44 illustrates a diagram of performing breath pattern analytics according to some embodiments.

FIG. 45 illustrates a flowchart of a method of performing passive analytics or active challenges prior to starting a new process or initiating a specific transaction according to some embodiments.

FIG. 46 illustrates a diagram of data points of a sensor plotted on a graph according to some embodiments.

FIG. 47 illustrates a diagram of a set of data points to be used to calculate a baseline according to some embodiments.

FIG. 48 illustrates a diagram of a calculated baseline according to some embodiments.

FIG. 49 illustrates a diagram of clusters of data points of location information according to some embodiments.

FIG. 50 illustrates a flowchart of a method of implementing a modified version of machine learning according to some embodiments.

FIG. 51 illustrates a flowchart of a method of implementing user movement and behavior tracking for security and suspicious activities according to some embodiments.

FIG. 52 illustrates a diagram of a system implementing user movement and behavior tracking for security and suspicious activities according to some embodiments.

FIG. 53 illustrates a flowchart of a method of implementing a bedside user device according to some embodiments.

FIG. 54 illustrates a diagram of a bedside user device and system according to some embodiments.

FIG. 55 illustrates a flowchart of a method of implementing an immediate health and mood monitoring system according to some embodiments.

FIG. 56 illustrates a flowchart of a method of implementing a long-term health and mood monitoring system according to some embodiments.

FIG. 57 illustrates a flowchart of a method of utilizing activity and behavioral analytics to enrich clinical drug trial data according to some embodiments.

### DETAILED DESCRIPTION OF THE PREFERRED EMBODIMENT

A security platform architecture is described herein. The security platform architecture includes multiple layers and utilizes a combination of encryption and other security features to generate a secure environment.

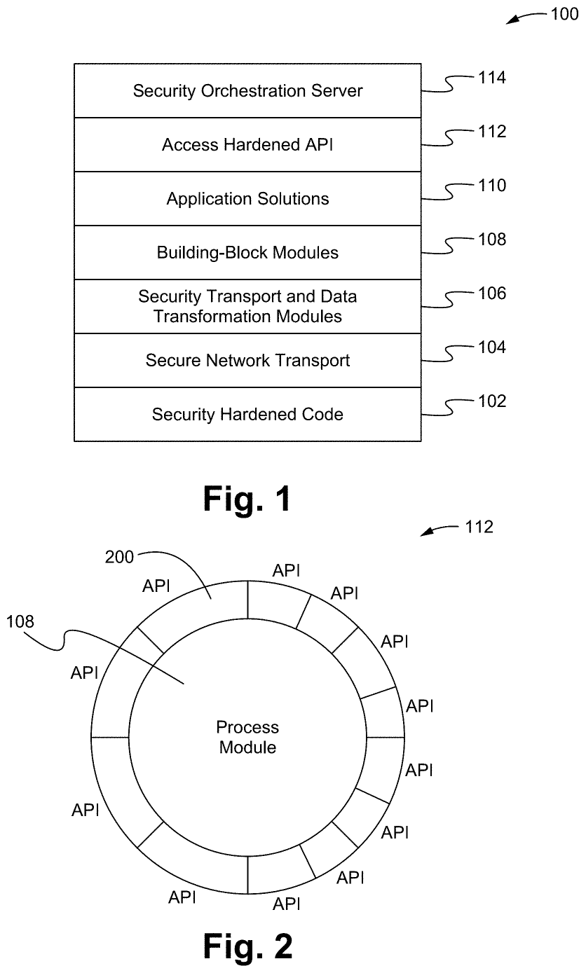

FIG. 1 illustrates a diagram of a security platform architecture according to some embodiments. The security platform 100 includes security-hardened code 102, secure network transport 104, security transport and data transformation modules 106, building block modules 108, application solutions/modules 110, access-hardened API/SDK 112, and a security orchestration server 114. In some embodiments, fewer or additional layers are implemented.

The security-hardened code 102 is able to include open or proprietary software security hardening. The security-hardened code 102 includes software libraries, executables, scripts, modules, drivers, and/or any other executable, accessible or callable data.

In some embodiments, the security-hardened code 102 is encrypted. For example, each library, executable and other data is encrypted. Furthering the example, an “encryption at rest” or “data at rest” encryption implementation is utilized. Data at rest encryption means data that is not in transit in a network or data that is not being executed is encrypted. Any data at rest encryption is able to be implemented including quantum encryption.

In some embodiments, the security-hardened code 102 is signed. For example, a digitally signed driver is associated with a digital certificate which enables identification of the publisher/owner of the driver.

In some embodiments, open or proprietary verification is based on encryption/decryption (e.g., the software modules/executables are inside an encrypted container), and is performed at installation and prior to each access. The security-hardened code 102 is fully tamper-proof. To be able to access the security-hardened code 102, a caller (e.g., calling module/procedure) should be part of the security domain.

In some embodiments, runtime verification of each executable, library, driver and/or data is implemented. Runtime verification is able to include any type of analysis of activity such as determining and learning keystrokes per user, or other mannerisms of computer interaction by each user.

In some embodiments, a security callback implementation is utilized. Before data is accessed or executed, the security callback calls to a master/server from the client, and if the hash or other verification implementation on the master/server does not match the hash/verification on the client, then access to the security-hardened code 102 is restricted/denied. For example, if a hash match fails, a software module will not be able to be executed, launched, moved or another action. The hash/verification comparison/analysis occurs before access of the security-hardened code 102. The security callback implementation is able to protect against instances where a virus or other malicious code has infiltrated a client device (e.g., mobile phone, personal computer).

The security-hardened code 102 is able to use any individual security technology or any combination of security technologies.

The security-hardened code 102 is able to be stored in a secure vault. The contents of the vault are encrypted using the data at rest encryption scheme. The contents of the vault are also signed. In some embodiments, white noise encryption is implemented which involves the use of white noise in the encryption. For example, white noise is generated using shift registers and randomizers, and the white noise is incorporated in the encryption such that if someone were to decrypt the content, they would obtain white noise.

The secure network transport 104 is able to be a high-speed, low-overhead, encrypted channel. In some embodiments, the secure network transport 104 uses quantum encryption (or post-quantum encryption). Quantum encryption is based on real keys (e.g., real numbers instead of integers) such that the encryption may not be hackable. Quantum encryption such as described in U.S. Provisional Patent Application No. 62/698,644, filed on Jul. 16, 2018, titled: “SECRET MATERIAL EXCHANGE AND AUTHENTICATION CRYPTOGRAPHY OPERATIONS,” and PCT Application No. PCT/US2019/041871, filed on Jul. 15, 2019, titled: “SECRET MATERIAL EXCHANGE AND AUTHENTICATION CRYPTOGRAPHY OPERATIONS,” which are both incorporated by reference herein in their entireties for all purposes, is able to be utilized herein.

In some embodiments, everything that communicates uses the secure network transport 104. For example, when a software module communicates with another software module, information is sent using the secure network transport 104.

The secure network transport 104 is able to utilize a proprietary or open Internet key exchange, Trusted Platform Module (TPM) key processing and storage, IoT key exchange, and/or optical/sonic/infrared/Bluetooth® key exchange.

The security transport and data transformation modules 106 implement “data in motion” encryption and “data at rest” encryption. In some embodiments, encryption is implemented while the data is being accessed/executed. The security transport and data transformation modules 110 include a tunneling module to tunnel the implementation inside Secure Sockets Layer (SSL)/Transport Layer Security (TLS) to enable the data to be utilized on any platform/browser/software/hardware/standard. The tunneling is able to be TLS quantum tunneling. The security transport and data transformation modules 106 include Application Programming Interfaces (APIs), keys, Public Key Infrastructure (PKI) modules, and/or other modules/structures.

The building block modules 108 include processes, services, microservices such as: AUTH, TRANS, LOG, ETRANS, BLUETOOTH, ULTRASONIC, and/or RF, which are implemented using objects (including functions or sub-routines). The building block modules 108 come from the software code/libraries and are able to communicate via the secure network transport 104.

The building block modules 108 are able to communicate between each other. In some embodiments, the module to module communications utilize Qrist encryption transport (or another encryption scheme) which isolates the modules from threats of hacks, viruses and other malicious entities. Qrist transport is high performance and low latency which requires almost no overhead. Since the building block modules 108 are pulled from the encrypted code/libraries, they are not typically visible in memory.

The building block modules 108 also have layered APIs (e.g., a specific API to communicate amongst each other). The APIs enable additional flexibility and extendability as well as providing a firewall (or micro-firewall) between every service to ensure transactions are coming from the right place (e.g., no man in the middle), the correct data is involved, and so on. The communications between the building block modules 108 are also able to be over HTTP. For example, a Web Application Firewall (WAF) is utilized, which applies specific rules for HTTP application communications.

The building block modules 108 are able to include executables (.exe), dynamic link libraries (.dll), configuration information, or other types of data/files (e.g., .so). The building block modules 108 are able to run in the background as background processes. The building block modules 108 are able to communicate through encrypted communications. The encrypted communications go through a transport such as Internet Protocol (IP), encrypted pipes in memory, Bluetooth® or another implementation. As described herein, the services are wrapped in APIs. The APIs implement REST (e.g., a very thin web server/client).

The application solutions/modules 110 are able to be developed using the building block modules 108. Exemplary applications include: encrypted email attachments, CyberEye multi-factor authentication, ID proofing, secure document signing (e.g., Docusign), secure electronic transactions, smart machines (e.g., autonomous vehicles), SAAS login, OpenVPN, blockchain login, blockchain support, high performance transaction services, electronic locks and E-notary. For example, since Docusign is relatively unsecure (e.g., anyone can sign the document), by combining Docusign with a CyberEye multi-factor authentication or another identification technology, it is possible to increase the security such that only the intended person is able to sign the document. More specifically, data at rest encryption is utilized to ensure the document is secure while stored, and the multi-factor authentication is used to ensure that the person signing the document is the desired target, and data in motion encryption is used to ensure the signed document is not tampered with and is received at the correct location.

The application solutions/modules 110 are able to be run/executed on any computing device such as a smart phone, a personal computer, a laptop, a tablet computer, a server, a dedicated smart device, a computer workstation, a server, a mainframe computer, a handheld computer, a personal digital assistant, a cellular/mobile telephone, a smart appliance, a gaming console, a digital camera, a digital camcorder, a camera phone, a portable music player, a mobile device, a video player, a video disc writer/player (e.g., DVD writer/player, high definition disc writer/player, ultra high definition disc writer/player), a television, a home entertainment system, an augmented reality device, a virtual reality device, smart jewelry (e.g., smart watch), a vehicle (e.g., a self-driving vehicle), IoT devices or any other suitable computing device.

The access-hardened API/SDK 112 includes similar security (e.g., encryption) as in the other modules. The access-hardened API/SDK 112 is able to utilize REST or another API (e.g., RPC). By implementing the access-hardened API/SDK 112, communication with the outside world is facilitated. For example, using a scripting language (e.g., javascript), an external application is able to communicate with the system.

The security orchestration server 114 is/includes a scripting language where when a call is received, the process goes down through the stacks starting at the top until the software library/code is reached (e.g., 114 through 102), and then the process goes up through the stacks out through the top (e.g., 102 through 114). Although the language is exposed to the outside world, it is based on the hardened code 102, so it is still secure.

The security orchestration server 114 accesses the security-hardened code 102 in the secure vault. The security orchestration server 114 includes keys and other information used for accessing the security-hardened code 102. The security orchestration server 114 deploys the services, builds keys, assigns commands/tasks and performs other control features. In some embodiments, the security orchestration server 114 organizes the building block modules 108 such that they are able to communicate with each other and function as an application 110.

When the security orchestration server 114 launches an application 110 (comprised of the building block modules 108), the security orchestration server 114 retrieves .dlls or other data and executes/communicates with the application 110 through the APIs of the building block modules 108.

The security orchestration server 114 controls deployment, policies and app structure. The app structure is also referred to as the application solutions/modules 110 which includes the code, the different modules/objects, and any data involved. The policies are able to be any policies such as for the firewall—what ports are open, which APIs are able to run in/with the application, who/what/when/where, well-structure calls (size of packets, and more), ports/ACL, and partners (which partners have access).

The secure orchestration server 114 implements a secure language such as python with extensions, java, and/or javascript.

In an example, a copy program is implemented by sending a copy command via the API which triggers a copy module which uses the transport scheme including data at rest encryption and data in motion encryption, and then goes to the transport layer and performs encryption/decryption, handles key exchanges and the copying using the code modules for copying.

FIG. 2 illustrates an exemplary access-hardened API according to some embodiments. The building block modules 108 enable communications and actions which are handled via RESTful APIs. Additionally, APIs 200 include Web Application Firewall (WAF) features to ensure that any communication between the building block modules 108 is secure/protected.

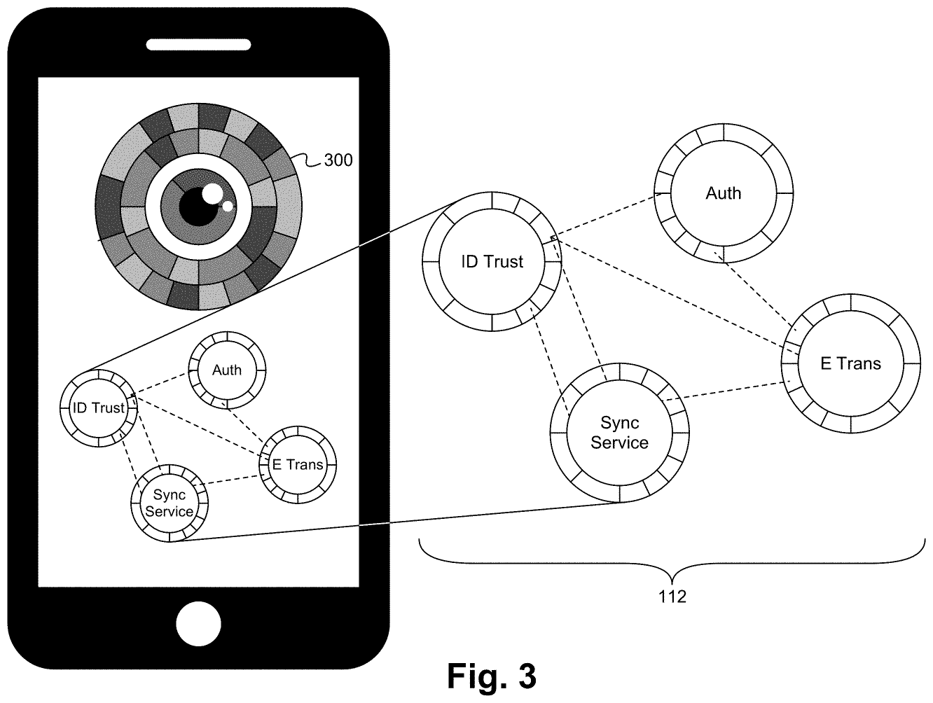

FIG. 3 illustrates a diagram of a secure application architecture according to some embodiments. An exemplary CyberEye implementation is able to be used to perform opti-crypto wireless airgap access (somewhat similar to a QR code). The building block modules 108 hardened by APIs 200 form the hardened APIs 112 which enable a modular services design, where each module is generalized for use in multiple application solutions. As described, the modules communicate with each other using encrypted communications (e.g., HTTP secure protocol). An API/WAF firewall is embedded in each module.

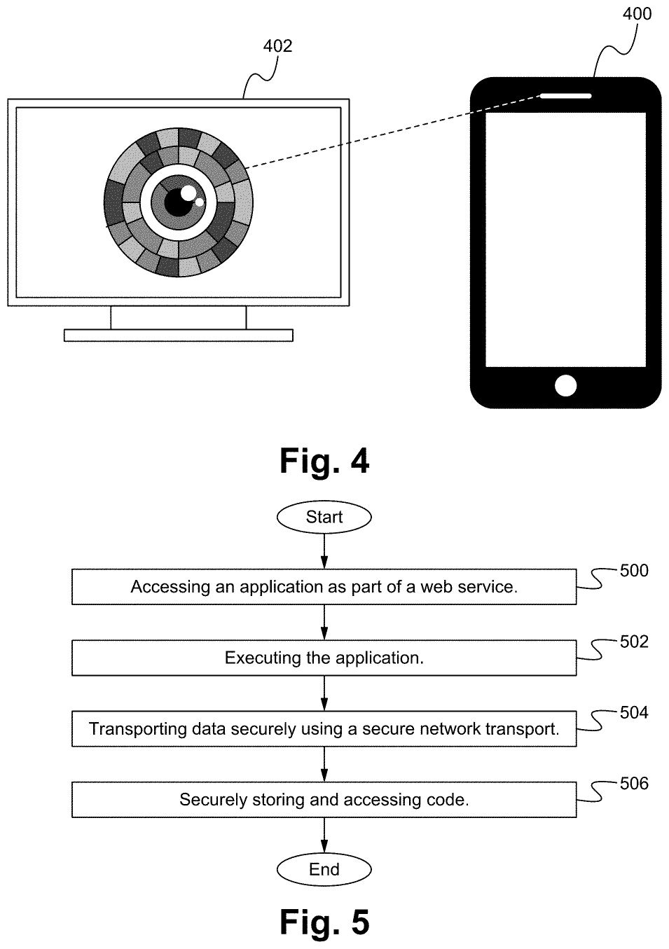

FIG. 4 illustrates a diagram of a smart device and a CyberEye multi-factor authentication according to some embodiments. As described in U.S. patent application Ser. No. 15/147,786, filed on May 5, 2016, titled: “Palette-based Optical Recognition Code Generators and Decoders” and U.S. patent application Ser. No. 15/721,899, filed on Sep. 30, 2017, titled: “AUTHENTICATION AND PERSONAL DATA SHARING FOR PARTNER SERVICES USING OUT-OF-BAND OPTICAL MARK RECOGNITION,” which are incorporated by reference herein in their entireties for all purposes, a smart device 400 (e.g., smart phone) is able to utilize an application (and camera) on the smart device 400 to scan a CyberEye optical recognition code mark displayed on another device 402 (e.g., personal computer or second smart device) to perform multi-factor authentication. As described herein, the CyberEye multi-factor authentication is an application module which is composed of building block modules which transport data securely using a secure network transport, where the building block modules are composed of software code which is securely stored and accessed on the smart device 400. The CyberEye multi-factor authentication is an example of an application executable using the security platform architecture.

FIG. 5 illustrates a flowchart of a method of implementing a security platform architecture according to some embodiments. In the step 500, an application is accessed as part of a web service such that a security orchestration server or access-hardened API is used to access the application. In the step 502, the application is executed. The application is composed of building block modules which transport data securely using a secure network transport, in the step 504. The building block modules are composed of software code which is securely stored and accessed on a device, in the step 506. Secure access involves data at rest encryption/decryption as well as data in motion encryption/decryption. In some embodiments, encryption/decryption involves quantum encryption/decryption using real numbers. In some embodiments, transporting the data includes utilizing tunneling such that the data is secure but also able to be transmitted over standard protocols. In some embodiments, fewer or additional steps are implemented. For example, in some embodiments, the application is a standalone application not accessed as part of a web service. In some embodiments, the order of the steps is modified.

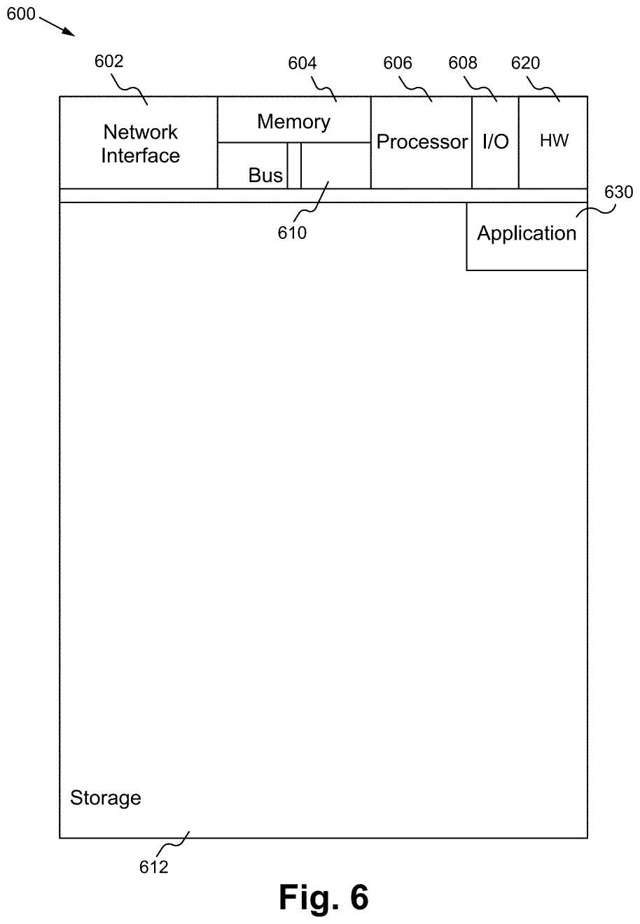

FIG. 6 illustrates a block diagram of an exemplary computing device configured to implement the security platform architecture according to some embodiments. The computing device 600 is able to be used to acquire, store, compute, process, communicate and/or display information. The computing device 600 is able to implement any of the security platform architecture aspects. In general, a hardware structure suitable for implementing the computing device 600 includes a network interface 602, a memory 604, a processor 606, I/O device(s) 608, a bus 610 and a storage device 612. The choice of processor is not critical as long as a suitable processor with sufficient speed is chosen. The memory 604 is able to be any conventional computer memory known in the art. The storage device 612 is able to include a hard drive, CDROM, CDRW, DVD, DVDRW, High Definition disc/drive, ultra-HD drive, flash memory card or any other storage device. The computing device 600 is able to include one or more network interfaces 602. An example of a network interface includes a network card connected to an Ethernet or other type of LAN. The I/O device(s) 608 are able to include one or more of the following: keyboard, mouse, monitor, screen, printer, modem, touchscreen, button interface and other devices. Security platform architecture application(s) 630 used to implement the security platform architecture are likely to be stored in the storage device 612 and memory 604 and processed as applications are typically processed. More or fewer components shown in FIG. 6 are able to be included in the computing device 600. In some embodiments, security platform architecture hardware 620 is included. Although the computing device 600 in FIG. 6 includes applications 630 and hardware 620 for the security platform architecture, the security platform architecture is able to be implemented on a computing device in hardware, firmware, software or any combination thereof. For example, in some embodiments, the security platform architecture applications 630 are programmed in a memory and executed using a processor. In another example, in some embodiments, the security platform architecture hardware 620 is programmed hardware logic including gates specifically designed to implement the security platform architecture.

In some embodiments, the security platform architecture application(s) 630 include several applications and/or modules. In some embodiments, modules include one or more sub-modules as well. In some embodiments, fewer or additional modules are able to be included.

In some embodiments, the security platform architecture hardware 620 includes camera components such as a lens, an image sensor, and/or any other camera components.

Examples of suitable computing devices include a personal computer, a laptop computer, a computer workstation, a server, a mainframe computer, a handheld computer, a personal digital assistant, a cellular/mobile telephone, a smart appliance, a gaming console, a digital camera, a digital camcorder, a camera phone, a smart phone, a portable music player, a tablet computer, a mobile device, a video player, a video disc writer/player (e.g., DVD writer/player, high definition disc writer/player, ultra high definition disc writer/player), a television, a home entertainment system, an augmented reality device, a virtual reality device, smart jewelry (e.g., smart watch), a vehicle (e.g., a self-driving vehicle), IoT devices or any other suitable computing device.

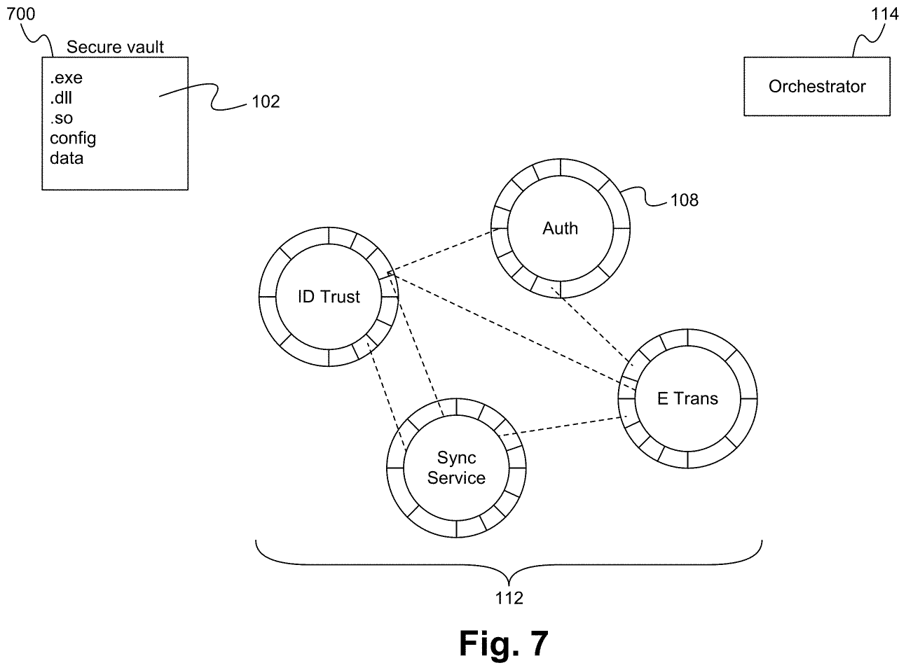

FIG. 7 illustrates a diagram of a secure application framework and platform according to some embodiments. The secure application framework and platform includes: a secure vault 700, a secure orchestration server 114 (also referred to as an orchestrator), and a set of building block modules 108 which form an application implemented via an access-hardened API 112. As described herein, the secure vault 700 stores the code 102 using encryption (e.g., white noise encryption) and signing, where the code 102 is used to generate/form the building block modules 108 which when organized form an application. The secure orchestration server 114 is able to control access to the code, deploy services, control one or more policies, and organize the one or more building block modules. Additional or fewer components are able to be included in the secure application framework and platform.

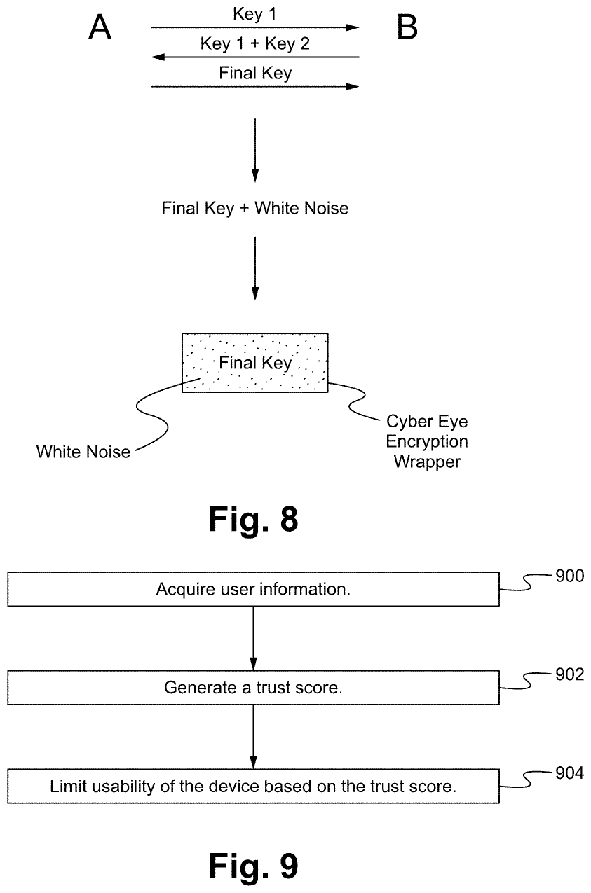

FIG. 8 illustrates a diagram of a secure key exchange through an opti-encryption channel according to some embodiments. Device A sends a first key to Device B, and Device B sends the first key and a second key back to Device A. Then Device A sends a final key to Device B, where the final key is based on the first key and the second key. In some embodiments, the final key is computed using the first key and the second key and one or more equations (e.g., linear equations). In some embodiments, white noise is inserted into the final key, or the final key is wrapped in white noise. In some embodiments, the keys are real numbers instead of integers.

In some embodiments, the final key is protected by optical encryption. As described herein, a user uses a camera device such as a camera on a mobile phone or tablet to scan/acquire a dynamic optical mark (e.g., CyberEye mark). The CyberEye result is wrapped around the final key. In some embodiments, the final key (with white noise) is encrypted/wrapped using the CyberEye encryption (or other opti-crypto wireless airgap encryption) information. In some embodiments, the opti-crypto key wrapper is a key encapsulation algorithm. In some embodiments, the optical encryption is used to generate the key. For example, the CyberEye result is a key or the final key which is combined with white noise.

Once the keys are passed, an encrypted communication/channel is able to be established (e.g., AES). In some embodiments, the encryption used is polymorphic, meaning the keys for the packets continuously change. In some embodiments, the encryption utilized with the encrypted communication/channel is post quantum encryption which enables quantum resistant encryption.

In some embodiments, a user's computing device is able to be used as a secure identification (e.g., ID proofing). The computing device is able to have a TPM or similar device/implementation for securing certificates. The TPM or similar implementation has break-in detection and other security measures. The computing device also includes machine learning implementations (processors/microchips). The computing device is able to include other standard components such as a CPU, one or more cameras, a screen, communication modules (e.g., Bluetooth,® WiFi, 5G, xG), and others.

ID proofing is able to prove/guarantee a user is who they claim to be. Instead of or in addition to biometric identification (e.g., fingerprint matching) and facial/voice recognition, other aspects of a user or a user's actions are able to be analyzed (e.g., behavior analysis). For example, a user's gate/stride, how the user uses his device, how the user types/swipes, and other motions/actions/transactions are able to be analyzed, compared and matched to determine if the user is the expected/appropriate user. Furthering the example, if a user typically takes short strides while using the phone and uses two thumbs to input text, then when a second user attempts to use the phone but has longer strides and uses a single finger input, then the device is able to detect that the person using the device is not the expected user (e.g., owner of the mobile phone).

A trust score is able to be generated based on the analysis. For example, as more matches are made (e.g., valid biometric input, matching stride, and matching typing performance, the trust score increases). Policies are able to implemented based on the trust score. For example, one or more thresholds are able to be utilized such that if the trust score is below a threshold, then options are limited for that user. Furthering the example, if a user has a 100% trust score, then there are no limitations on the user's use of the device, but if the user has a 50% trust score, below a money threshold, then the user is not able to perform any transactions involving money with the device, and if the user has a 5% trust score, the user is not able to access any applications of the device. Any number of thresholds are able to be used, and any limitations/consequences are able to be implemented based on the thresholds/trust score. The orchestrator described herein is able to implement these policies. In some embodiments, a risk score is implemented which is similar but inverse of the trust score.

In some embodiments, a transaction proxy is implemented. The transaction proxy is able to utilize the trust score to determine which transactions are allowed. The transactions are able to include any transactions such as logging in to a web site/social media, accessing an application (local/online), purchasing goods/services, transferring money, opening a door, starting a car, signing a document or any other transaction. In some embodiments, if a user's trust score is currently below a threshold, the device is able to perform additional tests of the user to increase their trust score (e.g., ask the user to say a word to determine a voice match). Passwords and personal information are able to be stored locally on the device (or on the Internet/cloud) for retrieval for access/comparison purposes. As described herein, the data (e.g., passwords and personal information) are able to be encrypted and backed up. For example, if the device is lost, the backup enables a user to purchase another device and retrieve all of the passwords/personal information.

In some embodiments, the implementation is or includes an extensible transaction method. For example, the device includes an application with a list of transactions (e.g., plug-ins). Once a transaction is initiated (e.g., Facebook login where Facebook password is pulled from the TPM), the transaction with all of the required information is stored as an encrypted file which is sent to a secure server proxy which is able to decrypt the file and then make the transaction. Since the transaction is able to occur using a proxy, the user is able to remain anonymous. In some embodiments, the opti-encryption implementation is able to be utilized with the secure identification implementation.

FIG. 9 illustrates a flowchart of a method of utilizing a user device as identification according to some embodiments. In the step 900, user information is acquired. The user information is able to be acquired in any manner such as receiving and logging keystrokes/touches from a keyboard/digital keypad/touch screen, measuring movement using an accelerometer or other device in a mobile device, acquiring imaging information using a camera (e.g., camera phone), acquiring voice information using a microphone, and/or any other implementation described herein.

In the step 902, a trust score is generated. The trust score is generated by analyzing the acquired user information. For example, an application records (and learns) how a user types, and compares how the current input with previous input to determine similarities. Similarly, the application is able to analyze a user's stride (long, short, fast, slow) by capturing the data over periods of time for comparison purposes. The trust score is also able to be based on other information such as location, time, device information and other personal information. For example, if the device is determined to be in Mexico, and the user has never visited Mexico previously, the trust score is able to be decreased. Or if the device is being used at 3 a, when the user does not use the device after 10 p or before 6 a, then the trust score is decreased.

In the step 904, usability of the device is limited based on the trust score. For example, if the trust score is below a minimum threshold, the user may be prevented from doing anything on the device. In another example, if the user's trust score is determined to be below an upper threshold, the user may be permitted to utilize apps such as gaming apps, but is not able to use the device to make purchases, sign documents or login to social media accounts. In some embodiments, actions/transactions are classified into classes or levels, and the classes/levels correspond to ranges of trust scores or being above or below specified thresholds. For example, purchases of $10 or more and signing documents are in Class 1, and Class 1 actions are only available when a trust score is 99% or above, and purchases below $10 and social media logins are in Class 2, and Class 2 actions are available when a trust score is 80% or above.

In some embodiments, fewer or additional steps are implemented. For example, if a user's trust score is below a threshold for an action that the user wants to take, the device is able to request additional proof by the user (e.g., provide a fingerprint and/or input a secret code) to increase the user's trust score. In some embodiments, the order of the steps is modified.

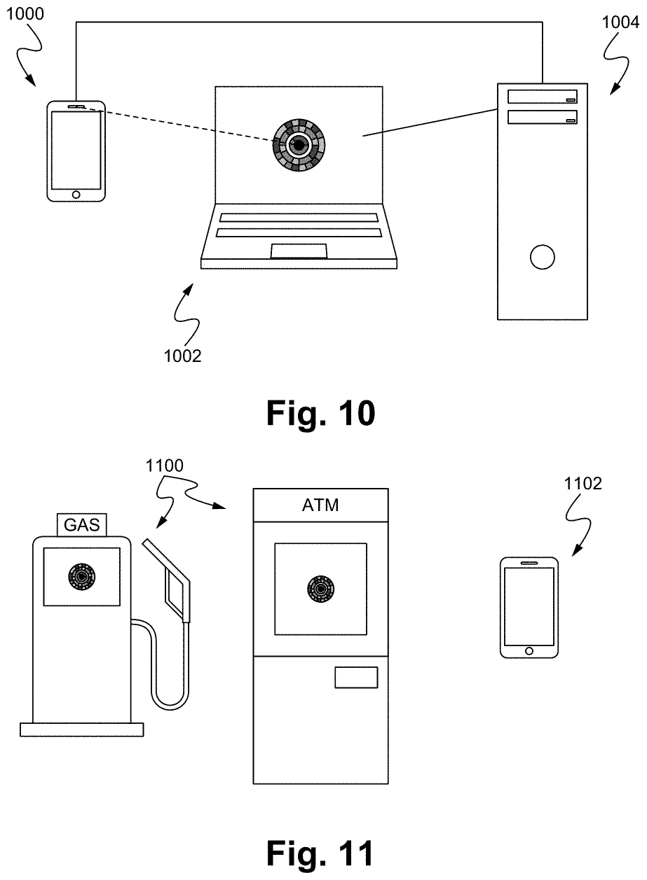

FIG. 10 illustrates a diagram of an optical encryption implementation according to some embodiments. As described herein, a device 1000 (e.g., smart phone) includes a camera which is able to acquire an image of a CyberEye implementation (e.g., repeating pattern) displayed in a web browser on another device 1002 (e.g., personal computer). The web browser is able to come from a server 1004 (e.g., local server). The server is able to provide authentication. There is also a back channel from the server to the device 1000. As described herein, the device 1000 is able to be used as a user's ID.

FIG. 11 illustrates a diagram of an optical encryption implementation on multiple devices according to some embodiments. The CyberEye implementation (or other optical multi-factor authentication) is able to be implemented on a gas station pump, Automated Teller Machine (ATM) machine, or any other device capable of displaying a multi-factor authentication implementation. For example, the gas station pump or ATM includes a display which is capable of displaying a web browser with a CyberEye implementation. The user is then able to use his mobile device to scan/acquire an image of the CyberEye, and then based on the ID proofing described herein, the user's device is able to authenticate payment or perform other transactions with the gas station pump, ATM or other device.

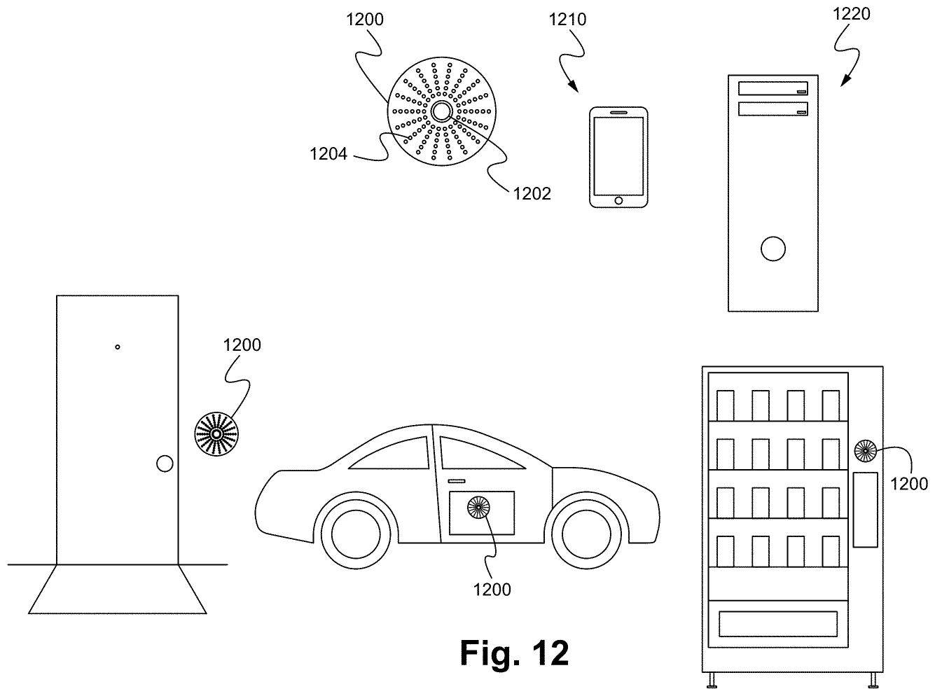

FIG. 12 illustrates a diagram of an optical encryption implementation on multiple devices according to some embodiments. In some embodiments, instead of or in addition to implementing a display with a CyberEye (or similar) implementation an embedded electronic device 1200 is utilized. The embedded electronic device 1200 includes a camera 1202 and lights 1204 (e.g., LEDs). In addition, other standard or specialized computing components are able to be included such as a processor, memory and a communication device (e.g., to communicate with WiFi).

In some embodiments, the embedded electronic device 1200 illuminates/flashes the lights 1204 in a specific pattern which a user device 1210 (e.g., smart phone) is able to scan/capture (similar to the CyberEye implementation). For example, upon the user device 1210 scanning the pattern provided by the embedded electronic device 1200, the user device 1210 (or the embedded electronic device 1200) sends an encrypted communication to perform a transaction. In some embodiments, a server 1220 determines (based on stored policies as described herein) whether the user's trust score is above a threshold to perform the transaction. For example, the user device 1210 is able to be used to unlock a house door, open a car door or purchase items at a vending machine. Furthering the example, in an encrypted communication to the server 1220 based on the scan of the embedded electronic device 1200, a transaction request to open the front door is sent to the server 1220 (either by the embedded electronic device 1200 or the user device 1210). The server 1220 compares the trust score with policies (e.g., if trust score is 99% or above, then unlock the lock; otherwise, no operation), and performs or rejects the requested transaction. For example, the server 1220 sends a communication to the embedded electronic device 1200 to unlock the lock of the door. The communication is able to be sent to a local or remote server for authentication which then communicates to the specific device (e.g., house door lock), or the communication is sent directly to the specific device (e.g., peer-to-peer communication). In some embodiments, the embedded electronic device 1200 sends the communication to a local or remote server for authentication, and then upon receiving authentication, the embedded electronic device 1200 performs the transaction. In some embodiments, the embedded electronic device 1200 communicates with the server (e.g., communicates the transaction request), and the user device 1210 communicates with the server (e.g., the user ID/trust score), and the server uses the information received from both devices to perform an action or to send a communication to perform an action, as described herein.

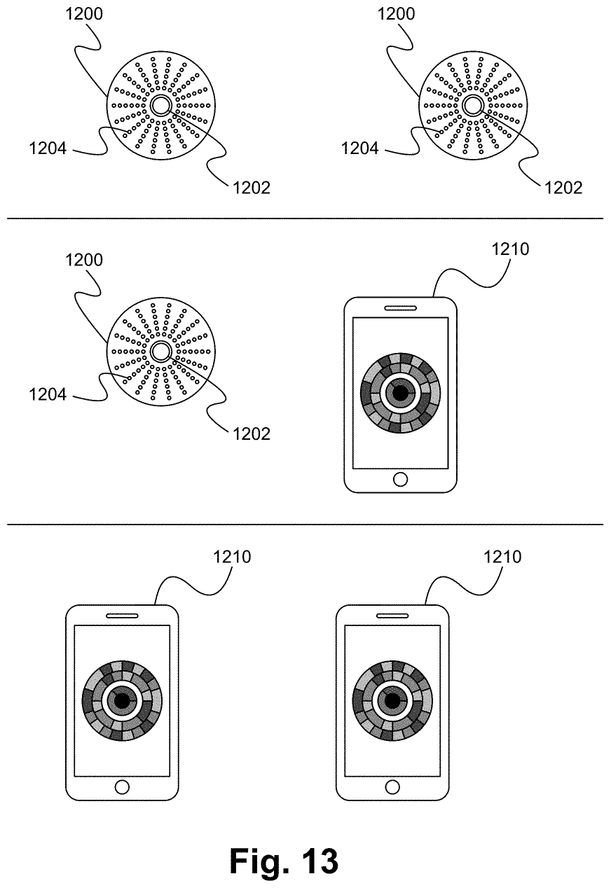

FIG. 13 illustrates a diagram of multiple embedded electronic devices and/or other devices according to some embodiments. In some embodiments, an embedded electronic device 1200 is able to communicate with one or more embedded electronic devices 1200. In some embodiments, an embedded electronic device 1200 is able to communicate with one or more other devices (e.g., user device 1210). In some embodiments, a user device 1210 is able to communicate with one or more other devices (e.g., user device 1210).

Since the embedded electronic device 1200 includes a camera 1202 and LEDs 1204, and a user device 1210 (e.g., mobile phone) includes a camera and a display to display a CyberEye (or similar) implementation, each is able to be used to display and acquire a unique code.

The multiple devices are able to communicate with each other and/or with a server. For example, a first user device is able to communicate with a second user device, and the second user device communicates with a server, and then provides the data received from the server to the first user device. Therefore, in some embodiments, the first user device (or embedded electronic device) does not need a connection with the server.

In some embodiments, the user device is able to replace a car key fob, since the user device is able to perform ID proofing as described herein, and is able to communicate with an embedded electronic device (e.g., a vehicle door lock/other vehicle controls). Similarly, with minimal modification, a car key fob is able to implement the technology described herein.

In some embodiments, instead of using optics for encryption (e.g., scanning a CyberEye implementation), other schemes are used such as infra-red, Bluetooth®, RFID, sonic, ultrasonics, laser, or RF/WiFi.

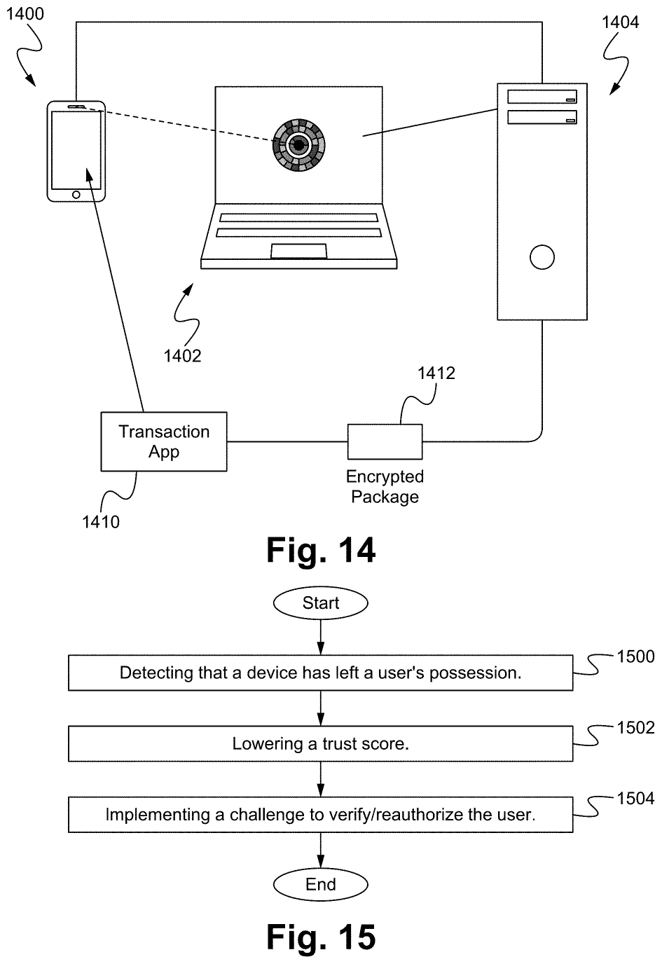

FIG. 14 illustrates a diagram of a system for electronic transactions using personal computing devices and proxy services according to some embodiments. A user device 1400 (e.g., smart phone) scans a CyberEye or similar implementation on a second device 1402 (e.g., personal computer or mobile device). The user device 1400 and/or the second device 1402 are able to communicate with a server 1404.

In some embodiments, the user device 1400 includes a transaction application 1410 programmed in memory. The transaction application 1410 is configured to send an encrypted package 1412 to the server 1404 based on the scan of the CyberEye or similar implementation (e.g., dynamic optical mark/code). The transaction application 1410 is able to trigger actions such as log in to a social media site, log in to a bank account, perform a monetary transfer, and/or any other transaction.

The server 1404 implements a proxy to perform the electronic transactions such as authentication, unlock door, moving money, e-signature and/or any other transaction. The transactions available through the transaction application 1410 are also added to the server 1404, such that the number of transactions is extensible. As described herein, the transactions are able to be accompanied by a trust or risk score such that if the trust/risk score is above or below a threshold (depending on how implemented), then the transaction request may be denied. By using the proxy to perform the electronic transactions, a user's anonymity and security is able to be maintained. With a transaction directly from a user device 1400, there is still potential for eavesdropping. However, as mentioned above, the transaction application 1410 sends an encrypted package/packet (e.g., token), which includes the transaction information (e.g., transaction ID, phone ID, trust score, specific transaction details such as how much money to transfer) to the server, where the proxy performs the transaction. The proxy server has secure connections to banks, Paypal, social networking sites, and other cloud servers/services. Furthermore, in some embodiments, the proxy server communication does not specify details about the user. In some embodiments, after the proxy server performs the transaction, information is sent to the user device. In some embodiments, the information sent to the user device is encrypted. For example, after the proxy server logs in to Facebook, the Facebook user page is opened on the user device.

In an example, a user receives a document to sign on the second device 1402. The user clicks the document icon to open the document, which then causes a CyberEye mark to appear. The user then scans the CyberEye mark with the user device 1400 which performs the ID proofing/authentication as described herein. The document is then opened, and it is known that the person who opened the document is the correct person. Similarly, the document is able to be signed using the CyberEye mark or a similar implementation to ensure the person signing the document is the correct person.

As described herein, a user device (e.g., mobile phone) is able to be used for ID proofing, where the user device recognizes a user based on various actions/input/behavioral/usage patterns (e.g., voice/facial recognition, stride/gate, location, typing technique, and so on). In some embodiments, potential user changes are detected. For example, if a user logs in, but then puts the device down, another user may pick up the phone, and is not the original user. Therefore, actions/situations such as putting the phone down, handing the phone to someone else, leaving the phone somewhere are able to be detected. Detecting the actions/situations is able to be implemented in any manner such as using an accelerometer to determine that the phone is no longer moving which would indicate that it was put down. Similarly, sensors on the phone are able to determine that multiple hands are holding the phone which would indicate that the phone is being handed to someone else. In some embodiments, the user device is configured to determine if a user is under duress, and if the user is under duress, the trust score is able to be affected. For example, an accelerometer of the user device is able to be used to determine shaking/trembling, and a microphone of the device (in conjunction with a voice analysis application) is able to determine if the user's voice is different (e.g., shaky/trembling). In another example, the camera of the user device is able to detect additional people near the user and/or user device, and if the people are unrecognized or recognized as criminals (e.g., face analysis with cross-comparison of a criminal database), then the trust score drops significantly (e.g., to zero).

As discussed herein, when a user attempts to perform an action/transaction where the user's trust score is below a threshold, the user is able to be challenged which will raise the user's trust score. The challenge is able to be a behavioral challenge such as walking 10 feet so the user device is able to analyze the user's gate; typing a sentence to analyze the user's typing technique; or talking for 10 seconds or repeating a specific phrase. In some embodiments, the user device includes proximity detection, fingerprint analysis, and/or any other analysis.

In some embodiments, an intuition engine is developed and implemented. The intuition engine continuously monitors a user's behavior and analyzes aspects of the user as described herein. The intuition engine uses the learning to be able to identify the user and generate a trust score.

With 5G and future generation cellular networks, user devices and other devices are able to be connected and accessible at all times, to acquire and receive significant amounts of information. For example, user device locations, actions, purchases, autonomous vehicle movements, health information, and any other information are able to be tracked, analyzed and used for machine learning to generate a behavioral fingerprint/pattern for a user.

In some embodiments, when a user utilizes multiple user devices, the user devices are linked together such that the data collected is all organized for the user. For example, if a has a smart phone, a smart watch (including health monitor), and an autonomous vehicle, the data collected from each is able to be stored under the user's name, so that the user's heart beat and driving routes and stride are able to be used to develop a trust score for when the user uses any of these devices.

To utilize the security platform architecture, a device executes an application which is composed of building block modules which transport data securely using a secure network transport, where the building block modules are composed of software code which is securely stored and accessed on the device. In some embodiments, the application is accessed as part of a web service such that a security orchestration server or access-hardened API are used to access the application. The security platform architecture is able to be implemented with user assistance or automatically without user involvement.

In operation, the security platform architecture provides an extremely secure system capable of providing virtually tamper-proof applications.

The security platform architecture implements/enables: a unique Opti-crypto wireless airgap transport, a personal smart device—intelligent ID proofing, secure extensible electronic transaction framework, blockchain integration and functionality, anonymous authentication and transaction technology, post quantum encryption at rest and in motion, secure private key exchange technology, secure encryption tunneled in TLS, high-throughput, low-latency transport performance, low overhead transport for low power FOG computing applications such as IOT, RFID, and others.

The security platform architecture is able to be utilized with:
Consumer applications such as games, communications, personal applications;
Public Cloud Infrastructure such as SAAS front-end security, VM-VM, container-container security intercommunications;
Private Cloud/Data Centers such as enhanced firewall, router, edge security systems; Telco Infrastructures such as CPE security, SDN encrypted tunnels, MEC edge security and transports, secure encrypted network slicing; and
5G New Market Smart Technologies such as smart machine security (sobots, autonomous vehicles, medical equipment).

The security platform includes infrastructure building blocks:
Client devices:
smart personal devices, IoT devices, RFID sensors, embedded hardware, smart machines;
Client functions:
ID proofing (trust analysis), CyberEye wireless transport, extensible electronic transaction clients, content and data loss security management, authorization client;
Transport functions:
Post-quantum data encryption technology, data-in-motion transport, data-at rest encryption, quantum tunnel through SSL/TLS, private-private secure key exchange, high-performance, low latency, low compute transport, TPM key management, SSL inspection;
Central server functions:
AAA services, federation gateway, electronic transactions server, adaptive authentication services, ID proofing services, user registration services, CyberEye transport server.

The security platform architecture is able to be used in business:
5G encrypted network slicing, electronic stock trading, vending machine purchasing interface, vehicle lock and security interfaces, anonymous access applications, Fog computing security transport (IoT to IoT device communications), SSL inspection security (decryption zones), generic web site/web services login services, MEC (mobile/multi-access edge gateway transport and security), cloud network backbone security firewalls (rack to rack FW), Office 365 secure login, low power IoT sensors, password management with single sign-on, high-security infrastructures requiring out-of-band or air gap enhanced access, or VM-to-VM (or containers) secure communications transport.

In some embodiments, device hand off identification proofing using behavioral analytics is implemented. For example, a device (e.g., mobile phone) detects when the device leaves a user's possession (e.g., put down on table, handed to another person). Based on the detection, when the device is accessed again, determination/confirmation that the user is the correct user is performed. In some embodiments, even if the device has not been placed in a locked mode (e.g., by a timeout or by the user), the device automatically enters a locked mode upon detecting leaving the user's possession.

FIG. 15 illustrates a flowchart of a method of device hand off identification proofing using behavioral analytics according to some embodiments. In the step 1500, a device detects that the device has left a user's possession. The device is able to be any device described herein (e.g., a mobile phone). Detecting that the device is no longer in the user's possession is able to be performed in any manner such as detecting that the device has been set down or handed off to another user. Other causes of a change in the user's possession are able to be detected as well such as a dropped device. In some embodiments, continuous monitoring of the device's sensors is implemented for detection, and in some embodiments, the sensors provide information only when triggered, or a combination thereof

Detecting the device has been set down is able to be performed using a sensor to detect that the device is stationary, using a proximity sensor, or any other mechanism. For example, one or more accelerometers in the device are able to detect that the device is in a horizontal position and is not moving (e.g., for a period of time above a threshold), so it is determined to have been set down. Determining the device has been set down is able to be learned using artificial intelligence and neural network training. For example, if a user typically props up his device when he sets it down, the general angle at which the device sits is able to be calculated/determined and recorded and then used for comparison purposes. In another example, the device includes one or more proximity sensors which determine the proximity of the device to another object. For example, if the proximity sensors detect that the object is immediately proximate to a flat surface, then the device has been determined to have been set down. In some embodiments, multiple sets of sensors work together to determine that the device has been set down. For example, the accelerometers are used to determine that the device is lying horizontally, the proximity sensors are used to determine that the device is proximate to an object, and one or more motion sensors detect that the device has not moved for 3 seconds. The cameras and/or screen of the device are able to be used as proximity sensors to determine an orientation and/or proximity of the device to other objects. The microphone of the device is able to be used as well (e.g., to determine the distance of the user's voice and the changes of the distances, in addition to possibly the distance and/or changes of distance of another person's voice). For example, if the user's voice is determined to be from a distance above a threshold (e.g., based on acoustic analysis), then it is able to be determined that the user has set the device down.

The process of setting a device down is able to be broken up and analyzed separately. For example, some users may place a device down in a certain way, while other users may make certain motions before putting the device down. Furthering the example, the steps of setting the phone down are able to include: retrieving the device, holding the device, moving the device toward an object, placing the device on the object, and others. Each of these steps are able to be performed differently, so breaking down the process of setting down the device in many steps may be helpful in performing the analysis/learning/recognition of the process. In some embodiments, the steps are, or the process as a whole is, able to be classified for computer learning. For example, one class of setting the phone down is labeled “toss,” where users throw/toss their device down which is different from “gentle” where users gently/slowly place their device down. The “toss” versus “gentle” classifications are able to be determined as described herein such as based on the accelerometer and/or gyroscope information. In another example, some users hold the device vertically before placing it down, while others hold it horizontally, or with one hand versus two hands. The classifications are able to be used for analysis/comparison/matching purposes. Any data is able to be used to determine the device being set down (e.g., movement, proximity, sound, scanning/video, shaking, touch, pressure, orientation and others) using any of the device components such as the camera, screen, microphone, accelerometers, gyroscopes, sensors and others.

Detecting the device has been handed off is able to be performed in any manner. For example, sensors on/in the device are able to detect multiple points of contact (e.g., 4 points of contact indicating two points from one user's hand and two points from a second user's hand, or a number of points above a threshold). In another example, the accelerometers and/or other sensors (e.g., proximity sensors) are able to analyze and recognize a handoff motion (e.g., the device moving from a first position and moving/swinging outward to a second position, or side-to-side proximity detection). In some embodiments, a jarring motion is also able to be detected (e.g., the grab by one person of the device from another person). The handoff motion/pattern is able to be learned using artificial intelligence and neural network training. In some embodiments, motions/movements from many different users are collected and analyzed to determine what movements are included in a handoff. Furthermore, each user's movements are able to be analyzed separately to determine a specific handoff for that user. For example, User A may hand off a device to another user in an upright position after moving the device from his pocket to an outreached position, while User B hands off a device in a horizontal position after moving the device in an upward motion from the user's belt.

Each separate aspect of the movement is able to be recorded and analyzed as described herein to compile motion information for further pattern matching and analysis. For example, the hand off motion is able to be broken down into separate steps such as retrieval of the device by a first person, holding of the device, movement of the device, release of the device, and acquisition of the device by the second person. Each of the separate steps are able to be recorded and/or analyzed separately. Each of the separate steps are, or the process as a whole is, able to be classified/grouped which may be utilized with computer learning and/or matching. Any data is able to be used to determine a handoff (e.g., movement, proximity, sound, scanning/video, shaking, touch, pressure, orientation and others) using any of the device components such as the camera, screen, microphone, accelerometers, gyroscopes, sensors and others.

Similarly, other changes of a user's possession are able to be detected such as the device being dropped. For example, the accelerometers are able to detect rapid movement followed by a sudden stop or slight reversal of movement. Similar to the hand off and set down, dropping and other changes of possession are able to be analyzed and learned.

In the step 1502, a trust score drops/lowers (e.g., to 0) after detection of a loss of possession. As described herein, the trust score of the user determines how confident the device is that the person using the device is the owner of the device (e.g., is the user actually User A). In some embodiments, factors are analyzed to determine the amount the trust score drops. For example, if the device is set down for a limited amount of time (e.g., less than 1 second), then the trust score is halved (or another amount of reduction). If the device is set down for a longer amount of time (e.g., above a threshold), then the trust score drops by a larger amount (or to 0). In another example, if the device is handed off, the trust score drops (e.g., to 0). In some embodiments, in addition to the trust score dropping, the device enters a locked/sleep mode.

In some embodiments, a device has different trust scores for multiple users. For example, if a family uses the same mobile phone—Mom, Dad, Son and Daughter each have different recognizable behaviors (e.g., motion/typing style) to determine who is currently using the phone. Each user has an associated trust score as well. For example, a device may have a trust score of 0 after being set down, but then after the device is picked up, it is determined that Mom is using the device, so her trust score is elevated (e.g., 100), but after a handoff, the trust score goes to 0, until it is determined that Dad is using the device, and his trust score is elevated (e.g., 100). In some embodiments, certain users have certain capabilities/access/rights on a device. For example, if the device detects Mom or Dad, then purchases are allowed using the device, but if Son or Daughter are detected, the purchasing feature is disabled.

In the step 1504, a challenge is implemented to verify/re-authorize the user. The challenge is able to include biometrics, a password request, a question challenge, favorite image selection, facial recognition, 3D facial recognition and/or voice recognition. In some embodiments, the device performs behavioral analytics as described herein to determine if the user is the owner/designated user of the device. For example, analysis is performed on the user's movements of the device, touch/typing techniques, gait, and any other behaviors. Based on the behavioral analytics, the trust score may rise. For example, if the behavioral analytics match the user's behaviors, then the trust score will go up, but if they do not match, it is determined that the device is being used by someone other than the user, and the trust score stays low or goes down. In some embodiments, the challenge enables initial access to the device, but the user's trust score starts low initially (e.g., 50 out of 100), and then based on behavioral analytics, the trust score rises.

In some embodiments, fewer or additional steps are implemented. In some embodiments, the order of the steps is modified.

In some embodiments, an automated transparent login without saved credentials or passwords is implemented. In the past, a device's browser could save a user's login and password information. However, this is a very vulnerable implementation, and once a hacker or other malicious person acquires the user's login and password information, the hacker is able to perform tasks with the user's account just as the user could, and potentially steal from an online bank account or make purchases on an online shopping site. Using a trust score and behavioral analytics, logging in to websites and other portals is able to be implemented automatically.

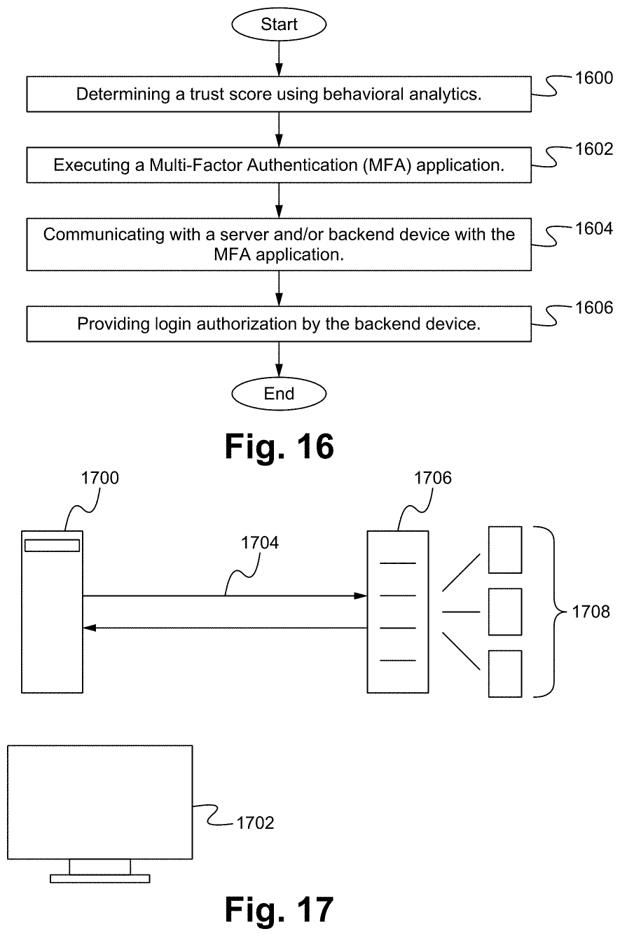

FIG. 16 illustrates a flowchart of a method of an automated transparent login without saved credentials or passwords according to some embodiments. In the step 1600, a trust score is determined using behavioral analytics as described herein. For example, based on user movement, typing style, gait, device possession, and so on, a trust score is able to be determined. Furthering the example, the closer each analyzed aspect of the user (e.g., gait) is to the stored user information, the higher the trust score. In another example, if the user typically types on his device using his thumbs, and the current person using the device is using his index finger, then the trust score is adjusted (e.g., lowered). In contrast, if the user has a distinct gait (e.g., typically walks with the device in his hand, while he swings his arms moderately), and the device detects that the current person walking with the device in his hand while swinging his arms moderately, the trust score increases.

In some embodiments, in addition to a trust score, a confidence score is determined for the user/device. In some embodiments, the confidence score for a user is based on the trust score and a risk score. In some embodiments, the risk score is based on environmental factors, and the trust score is based on behavioral factors. In some embodiments, the confidence score goes up when the trust score goes up, and the confidence score goes down when the risk score goes up. Any equation for the confidence score is possible, but in general as the trust increases, the confidence increases, but as the risk increases the confidence decreases.

In the step 1602, a multi-factor authentication (MFA) application is executed. The MFA application is able to be running in the foreground or the background. The MFA application is able to be implemented in a secure, isolated space as described herein to prevent it from being compromised/hacked. In some embodiments, the MFA application includes aspects (e.g., operations) to acquire information to determine the trust, risk and confidence scores. For example, the trust score and risk scores each have multiple factors which go into determining their respective scores which are used to determine the confidence score which is further used for authenticating a user.

In some embodiments, the MFA application utilizes the confidence score analysis and additional user verification implementations. For example, CyberEye (also referred to as CypherEye) application/technology is able to be executed with the device. In some embodiments, the MFA application and/or CypherEye application is used as a login authority. The MFA login or CypherEye login looks like a local login, but instead a hash (or other information) is sent to a backend mechanism. In some embodiments, the MFA application uses the CypherEye information in conjunction with the confidence score. In some embodiments, a challenge is implemented (e.g., a request for the user to perform a CypherEye operation) for additional verification/qualification. For example, if a user's confidence score is below a threshold, then the user is challenged with a CypherEye request to acquire a CypherEye mark with his device. In another example, a user is able to log in using the MFA application which gives the user access to basic phone functions (e.g., using Facebook), but to access banking/trading applications or web sites, the user is presented a challenge (e.g., security question, password, CypherEye acquisition using camera) for further verification.

In some embodiments, the challenge is only presented if the confidence score is not above a threshold. For example, if the user has a confidence score of 99 out of 100 on the device, then the user is not requested to perform additional authentication measures to gain access to web sites or applications. However, if the user has a confidence score of 50 out of 100, then additional authentication measures are utilized before access is given to certain web sites or applications. For example, although the user logged in using the MFA application, the device or system determined that the same user logged in (or attempted to) using a different device 500 miles away. The risk score is elevated since one of the log in attempts was likely not from a valid user, so the confidence score was lowered. A challenge may be presented in this situation.

In some embodiments, the MFA application is used in conjunction with a login/password. For example, a browser presents a web page for a user to input login information and a corresponding password as well as MFA information (e.g., a scanned CypherEye code/mark).

In some embodiments, the MFA application is a plugin for the browser.

In the step 1604, the MFA application (or plugin) contacts a server and/or backend device (e.g., Visa or PayPal) based on the MFA information (e.g., behavioral information or other acquired information). For example, the MFA application sends the confidence score as determined. In another example, the MFA application sends the acquired information to the server for the server to determine the confidence score. In some embodiments, the confidence score is utilized by the server such that if the confidence score is above a threshold, the server contacts the backend device with the user login information. Furthering the example, the server stores user login/password information to the backend device, and once the user is verified by the server based on the MFA information, then the server communicates the login/password information with the backend device to gain access for the user device. The MFA application and/or the server are able to implement a proxy authentication or other implementation to gain access to the backend device. In some embodiments, the MFA application acts as a proxy server, if the confidence score of the user is above a threshold (e.g., 90 out of 100).

In the step 1606, login authorization is provided by a backend device (e.g., allow the user to access a web page populated with the user's specific information (e.g., bank account information)). For example, the server (or proxy server) provides a login request with the appropriate credentials, and the backend device accepts the request and allows access to the service, or rejects the request and denies access to the service. In some embodiments, the server sends a hash or other code which identifies the user and indicates the user has been validated/authorized by the server to the backend device, and in some embodiments, the server sends identification information and verification information to the backend device, and the backend device performs the verification/authentication. In some embodiments, fewer or additional steps are implemented. In some embodiments, the order of the steps is modified.

FIG. 17 illustrates a diagram of a system configured for implementing a method of an automated transparent login without saved credentials or passwords according to some embodiments. A device 1700 utilizes an authentication implementation (e.g., MFA) to ensure a confidence score of the user is above a threshold (e.g., the device is confident that the user is who he says he is). In some embodiments, the authentication information is based on the confidence score, and if the confidence score is above a threshold, no further information is needed, meaning the user does not need to enter login/password information or additional MFA information (e.g., satisfy a challenge). As described herein, the user's device with a confidence score above a threshold identifies the user as the correct user.

In some embodiments, MFA includes behavioral analytics, where the device continuously analyzes the user's behavior as described herein to determine a trust score for the user. The device (or system) determines a risk score for the user based on environmental factors such as where the device currently is, previous logins/locations, and more, and the risk score affects the user's confidence score. In some embodiments, the scan of a dynamic optical mark is only implemented if the user's trust score (or confidence score) is below a threshold. For example, if a user has been continuously using his device as he normally does, his gait matches the stored information, and his resulting trust score is 100 (out of 100) and there have been no anomalies with the user's device (e.g., the risk score is 0 out of 100), then there may be no need for further authentication/verification of the user.

In some embodiments, the authentication implementation utilizes additional MFA information. For example, for additional MFA information, the user utilizes the device's camera to scan a dynamic optical code/mark which is displayed on a secondary device 1702. In another example, a challenge requests the user to input a login and password for a site (e.g., a bank site).

After a user attempts to log in (e.g., clicks a link/button to log into a banking web page), the device 1700 sends a communication (e.g., an access/login request) via a quantum resistant encryption transport 1704 (or another transport) to a server device 1706. The server device 1706 then communicates the request/authentication information to a backend device 1708 (e.g., company device) which provides access to the desired services/information (e.g., log in to a web page with bank account information). Depending on the implementation, different information may be sent from the device 1700 to the server device 1706, and from the server device 1706 to the backend device 1708. For example, the device 1700 may send the acquired MFA information and/or a confidence score to the server device 1706. In another example, the server device 1706 may send a hash for access for a specific user login. The server device 1706 may send the login information and an associated request possibly accompanied by the confidence score. The server device 1706 may send any other data to trigger an access request for a specific user, including or not, an indication that the user should gain access to the backend service/device. The server device 1706 and the backend device 1708 are able to communicate in any manner, using any standard, and via any APIs.

The backend device 1708 is able to utilize standard login/access protocols such as OATH2, SAML, Kerberos and others. The backend device 1708 provides the login authorization (or not) back to the server device 1706 depending on the authentication information. The server device 1706 provides the authorization acceptance to the device 1700 enabling access to the web page. In some embodiments, the server device 1706 acts as a proxy server as described herein. In some embodiments, the server device 1706 performs the authentication verification and does not send the request to the backend device 1708 unless the authentication verification is determined to be true (e.g., user is verified as authentic). In some embodiments, the backend device 1708 communicates the authorization directly with the device 1700. In some embodiments, the implementation described herein is a single sign-on mechanism. By utilizing MFA as described herein, a user will no longer need to store login and password information in his browser.

In some embodiments, automated identification proofing using a random multitude of real-time behavioral biometric samplings is implemented. Single behavioral analysis is susceptible to hacking or spoofing with pre-recorded or eavesdropped data. For example, human speech may be recorded surreptitiously; or human motions (e.g., gait) may be recorded from a compromised personal device or hacked if stored on a central source. Using multiple behavioral biometric mechanisms, sampled randomly, is much more difficult to spoof The larger number of biometric sensors and analytics employed greatly increases the security for authentication against either human hacking or robotic threats.

As described herein, Multi-Factor Authentication (MFA) is able to be based on possession factors, inheritance factors, and knowledge factors.

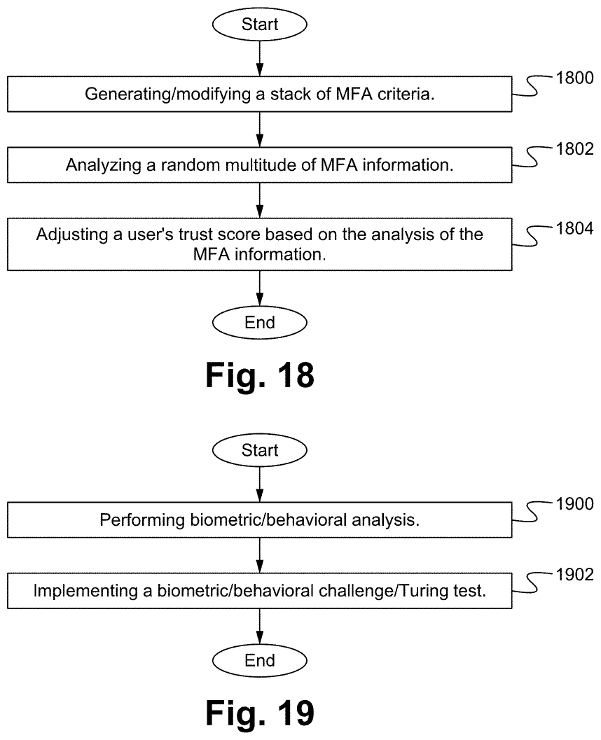

FIG. 18 illustrates a flowchart of a method of implementing automated identification proofing using a random multitude of real-time behavioral biometric samplings according to some embodiments. In the step 1800, a stack (or other structure) of MFA criteria is generated or modified. MFA information is able to be stored in a stack-type structure such that additional MFA criteria are able to be added to the stack. For example, initially, MFA analysis utilizes voice recognition, facial recognition, gait and typing style. Then, fingerprints and vein patterns are added to the stack so that more criteria are utilized for determining a trust score of a user. In some embodiments, a user selects the MFA criteria, and in some embodiments, a third party (e.g., phone maker such as Samsung, Apple, Google, or a software company or another company) selects the MFA criteria. The stack of MFA criteria is able to be modified by removing criteria. For example, if it has been determined that a user's fingerprint has been compromised, then that criterion may be removed and/or replaced with another criterion for that user.

In the step 1802, a random multitude of MFA information is analyzed. The MFA information is able to be based on: possession factors, inheritance factors, and knowledge factors. Possession factors are based on what the user possesses (e.g., key card, key FOB, credit/debit card, RFID, and personal smart devices such as smart phones, smart watches, smart jewelry, and other wearable devices). The personal smart devices are able to be used to perform additional tasks such as scanning/acquiring a dynamic optical mark/code using a camera. Inheritance factors are based on who the user is (e.g., biometrics such as fingerprints, hand scans, vein patterns, iris scans, facial scans, 3D facial scans, heart rhythm, and ear identification, and behavioral information such as voice tenor and patterns, gait, typing style, web page selection/usage). Knowledge factors are based on what a user knows (e.g., passwords, relatives' names, favorite image, previous addresses and so on).

Analysis of the MFA criteria is as described herein. For example, to analyze a user's gait, the user's gait information is stored, and the stored data points are compared with the current user's gait using motion analysis or video analysis. Similarly, a user's typing style is able to be captured initially during setup of the device, and then that typing style is compared with the current user's typing style. The analysis of the MFA criteria is able to occur at any time. For example, while the user is utilizing his device, the device may be analyzing his typing style or another criterion (possibly without the user knowing). Additionally, there are particular instances which trigger when the MFA criteria is analyzed, as described herein. For example, when it is detected that the device has left the user's possession, MFA analysis is performed upon device use resumption.

In some embodiments, the stack includes many criteria, but only some of the criteria are used in the analysis. For example, although 6 criteria are listed in a stack, the user has not provided a fingerprint, so that criterion is not checked when doing the analysis.

The MFA analysis is able to include challenges based on the trust score and/or an access request. Multiple thresholds are able to be implemented. For example, if a user's trust score is below 50%, then to perform any activities using the device, the user must solve a challenge (e.g., input a password, select a previously chosen favorite image, provide/answer another personal information question). Answering/selecting correctly boosts the user's trust score (the boost is able to be a percent increase or to a specific amount). In another example, if the user's trust score is above 50% but below 90%, the user is able to access lower priority applications/sites, but would be required to answer one or more challenges to raise the trust score above 90% to access high priority applications/sites such as a bank web site. In some embodiments, the trust score is part of a confidence score, and if the confidence score is below a threshold, then a challenge may be implemented.

In some embodiments, the analysis includes randomly sampling the MFA criteria. For example, although the MFA criteria stack may include eight criteria, each criterion is sampled in a random order. Furthering the example, when a user accesses his device, the user may be asked to provide a fingerprint, but then the next time he accesses his device, the user's gait is analyzed, and the next time, the user's typing style is analyzed, and so on. Any randomization is possible. In some embodiments, multiple criteria are analyzed together (e.g., typing style and fingerprints). In some embodiments, all of the criteria in a stack are utilized but are analyzed in a random fashion/order. For example, when a user accesses a device, he is required to input a password/PIN, then while the user is typing, his typing style is analyzed, and while the user is walking his gait is analyzed, but if the user starts typing again, his typing style is analyzed, and every once in a while a retina scan is requested/performed. The analysis of the criteria is able to be performed in any random order. In another example, sometimes when a user attempts to gain access to a device, he is prompted to provide a fingerprint, other times a password or PIN is requested, and sometimes a retinal scan is implemented. By changing the criteria being analyzed, even if a hacker has the user's password, if the hacker does not have the user's fingerprint or retina scan, their attempt to gain access will be thwarted. As described herein, in some embodiments, multiple criteria are utilized in combination at the same time or at different times.

In the step 1804, a user's trust score is adjusted based on the analysis of the MFA information. As described herein, the user's trust score goes up, down or stays the same based on the MFA information analysis. For example, if a current user's gait matches the stored information of the correct user's gait, then the user's trust score goes up (e.g., is increased). If the current user's typing style is different than the stored information of the correct user, then the user's trust score goes down (e.g., is decreased).

The amount that the trust score is adjusted is able to depend on the implementation. In some embodiments, the effect on the user's trust score is able to be absolute or proportional. For example, in some embodiments, if one criterion out of eight criteria is not a match, then the user's trust score drops significantly (e.g., by 50% or to 0). In another example, in some embodiments, if one criterion of eight is missed, then the trust score drops proportionately (e.g., by 1/8^{th}). In another example, the amount of the drop may depend on how close the currently acquired information is when compared to the stored information. For example, using comparative analysis, a user's gait is a 97% match with the stored information, so the trust score may drop slightly or not at all since the match is very close, whereas a match of 50% may cause a significant drop in the trust score (e.g., by 50% or another amount). When utilizing MFA criteria, if a user's current analysis results in a mismatch (e.g., the user has a different gait), then the user's trust score is lowered, even if the other criteria are matches. For example, seven of eight criteria are matches, but one of the criterion is a mismatch. In some embodiments, one mismatch significantly affects the user's trust score, and in some embodiments, the device/system is able to account for the fact that seven of eight criteria were matches, so the drop in the trust score may be minimal or proportionate. For example, one mismatch out of seven reduces the trust score by less than one mismatch out of two. In some embodiments, if there is one mismatch out of many criteria, the user may be prompted as to why there was a mismatch (e.g., an injury could cause the user to change his gait), and/or another criterion may be utilized.

As described herein, the trust score of the user for a device is able to be used as part of a confidence score (e.g., the confidence score is based on the trust score and a risk score). The confidence score is then used to determine whether the device or system has confidence that the user is who he says he is and what applications/sites the user has access to. A mismatch in the analysis criteria affects the confidence score, and based on the confidence score, additional factors/criteria may be analyzed and/or additional challenges may be utilized. In some embodiments, fewer or additional steps are implemented. In some embodiments, the order of the steps is modified.

In some embodiments, user identification proofing is implemented using a combination of user responses to system Turing tests using biometric methods. For example, device and/or system determines if the user is the correct user (e.g., the user is who he says he is) and is the user a human (and not a bot).

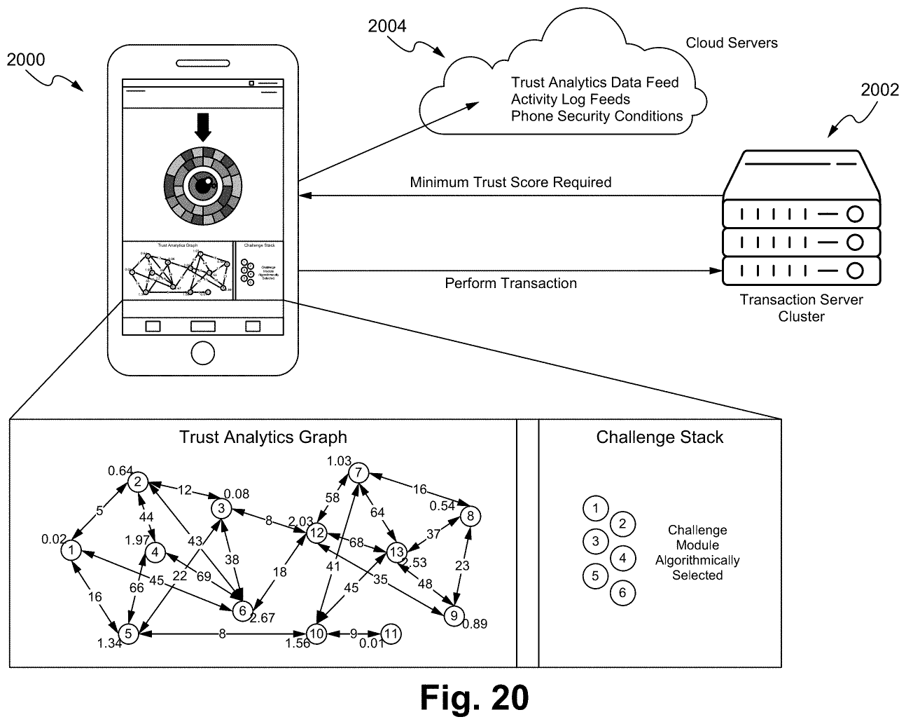

FIG. 19 illustrates a flowchart of a method of implementing user identification proofing using a combination of user responses to system Turing tests using biometric methods according to some embodiments.

In the step 1900, biometric/behavioral analysis is performed. Biometric analysis is able to be implemented as described herein and include analyzing: fingerprints, hand scans, vein patterns, iris scans, facial scans, 3D facial scans, heart rhythm, ear identification and others, and behavioral analysis is able to include analysis of information such as voice tenor and patterns, gait, typing style, web page selection/usage and others. For example, the device utilizes sensors, cameras, and/or other devices/information to scan/acquire/capture biometric and/or behavioral information for/from the user. The biometric/behavioral analysis is able to include comparing acquired information (e.g., fingerprints) with stored information (e.g., previously acquired fingerprints) and determining how close the information is and whether there is a match. Any implementation of comparison/matching is able to be implemented.

In the step 1902, a biometric/behavioral challenge/Turing test is implemented. For example, a user is requested to turn his head a certain direction or look a certain direction. Furthering the example, the user is prompted by the device to look up and then look right, and the camera of the device captures the user's motions and analyzes the user's motions using video processing implementations to determine if the user looked in the correct directions. In another example, voice recognition is able to be implemented including asking a user to repeat a specific, random phrase (e.g., a random set of word combinations such as “kangaroo, hopscotch, automobile”). The vocal fingerprint and the pattern of how a user talks are able to be analyzed. For example, the device/system is able to detect computer synthesized phrases by detecting changes in pitch, odd gaps (or a lack of gaps) between words, and other noticeable distinctions. Other actions are able to be requested and analyzed as well such as requesting the user to skip, jump, walk a certain way, and so on.

In some embodiments, the biometric/behavioral challenge/Turing test is related to the biometric/behavioral analysis (e.g., in the same class/classification). For example, if the biometric/behavioral test involves facial recognition, then then the biometric/behavioral challenge/Turing test is related to facial recognition such as requesting the user to turn his head in one or more specific directions. In some embodiments, the challenge/test is unrelated to the biometric/behavioral analysis (e.g., in a different class/classification). For example, if there is a concern that a user's facial recognition information has been compromised (e.g., detection of the same facial information within a few minutes in two different parts of the world), then the challenge/test is something unrelated to that specific biometric/behavioral analysis. Furthering the example, instead of asking the user to look a specific direction, the user is requested to speak a randomly generated phrase/sequence of words or to perform an action (e.g., jump, specific exercise). Exemplary classes/classifications include a facial/head class, a gait class, a speech/voice class, a typing class, and others.

The device utilizes sensors, cameras, and/or other devices/information to scan/acquire/capture biometric and/or behavioral information for/from the user to perform the challenge/Turing test. For example, the sensors/cameras capture user information and compare the user information with stored user information to determine if there is a match. In some embodiments, computer learning is able to be implemented to perform the analysis. For example, using computer learning, the analysis/matching is able to be implemented on possible iterations that were not specifically captured but are able to be estimated or extrapolated based on the captured information. In some embodiments, the challenge/Turing test is only implemented if the user passes the biometric/behavioral analysis. In some embodiments, the device (e.g., mobile phone) implements the analysis and challenge/test steps, and in some embodiments, one or more of the steps (or part of the steps) are implemented on a server device. For example, the device acquires the biometric and/or behavioral information which is sent to a server device to perform the analysis of the acquired biometric/behavioral information. Similarly, a response by a user to the challenge/Turing test is able to be acquired by a user device, but the acquired information is able to be analyzed on the server device.

In some embodiments, fewer or additional steps are implemented. For example, after a user is verified using the analysis and challenge/Turing test, the user is able to access the device and/or specific apps/sites using the device. In another example, after a user is verified using the analysis and challenge/Turing test, the trust score, and in conjunction, the confidence score of the user increases. In some embodiments, the order of the steps is modified.

Within an aggregated trust framework, there are analytics and challenges. The analytics are able to include multi-stage analytics including a weighted decision matrix, decision theory, decision tree analytics and/or others. However, scalability is an important factor when implementing the aggregated trust framework. For example, a tree structure is able to be used, but it involves rebalancing as elements are added to the structure. Thus, the structure to be used should be a scalable structure such as a matrix or a weighted table.

Included in the analytics are several steps/phases/modules. There is the base phase which runs in the background. A pre-transaction phase, an external/environmental phase, a device phase, and a hijack phase are also included. The analytics are able to include fewer or additional phases. The challenges are able to be included in the analytics or grouped separately. Each of the analytics and challenges is able to include sub-steps/sub-phases/sub-modules. For example, the base phase module includes a facial recognition sub-module, a voice recognition sub-module and a gait detection sub-module.

The base phase performs many analytical steps in the background (e.g., always running) such as performing an image/video scan of the user's face/body, analyzing the user's gait, and/or other analysis. For example, a device's camera is able to continuously scan the user, the surroundings, objects the user is holding, other objects near the user and/or anything else. In another example, the microphone of the device is able to continuously listen to a user's voice to perform voice analysis and detect changes in the user's voice (e.g., pattern, volume, pitch). In yet another example, the sensors of the device are able to detect specific movements of the user (e.g., gait), hand movements, grip strength, grip positioning, micro-tremors, swiping patterns, touch/typing/texting patterns, and/or others. The base phase is able to implement the various sub-phases simultaneously and switch the focus amount for them when one or more are applicable or inapplicable. For example, if the user has his smart phone in his pocket, the facial recognition aspect is not going to detect a user's face, so the voice recognition and gait detection aspects are continued to be utilized/analyzed.

An aggregate score (e.g., 0 to 100 or 0% to 100%) is able to be computed based on the base phase analytics. For example, the aggregate score is able to increase as correct/matching analytics are detected. For example, if the user's gait, voice, face and swiping movements match previously analyzed information, then the aggregate score may be 100; whereas, if the person detected is walking differently, has a different voice and face, and swipes differently than the previously analyzed information, then the aggregate score may be 0. The previously analyzed information is able to dynamically change as the learning of the user by the device continues. For example, the system does not merely ask the user to take a single scan or image of their face and use that for facial recognition. Rather, the system continuously acquires multiple face scans/images, and using artificial intelligence and machine learning, generates a large body of analytical information to be compared with the user's face. By having a large body of analytical information, if the user wears a hat one day or grows out his beard, then the system is still able to recognize the user as the user.

In some embodiments, if the aggregate score of the base is below a threshold (e.g., 60), then the pre-transaction phase analysis is implemented. The pre-transaction analysis is able to include additional analysis/testing to modify the aggregate score. For example, if the aggregate score is 55 which is below the threshold of 60, then the device performs a facial recognition scan, which if a match is detected, then the aggregate score is increased by 10 such that the aggregate score is above the threshold. With the aggregate score above the threshold, a transaction is able to occur. In some embodiments, the pre-transaction phase includes analytics that are different from the base phase analytics.

The external/environmental phase analyzes external or environmental factors such as the device's location and ambient information (e.g., temperature, lighting, barometer/altimeter information). For example, if the user lives in California, but the phone or communication is determined to be located/coming from China, then the aggregate score would be negatively affected (e.g., dropped to 0 or reduced to below a threshold). In another example, the device determines that the user is using the device at midnight with the lights off, and this is atypical behavior based on previous external/environmental analysis, so the aggregate score is negatively affected.

The device phase analyzes the device information to protect against a computer-based attack. For example, the device is behaving oddly or the system has been spoofed and is being implemented/accessed on a different device than was originally analyzed. Similarly, malware is able to infect a user's device and trigger inappropriate transactions. Therefore, the device phase is able to perform system checks such as a virus scan, a malware scan, a hardware/system check, an OS check, and/or any other device check/analysis. The device phase is also able to affect the aggregate score. For example, if a hardware check is performed, and it is determined that the hardware is different from the original hardware when the app first performed a hardware check, then the aggregate score drops to 0.

The hijack phase analyzes possible change of possession of the device. For example, when a user hands the device to another user, or when the user places the device down, then another user may be in possession of the device. Again, the hijack phase is able to affect the aggregate score. For example, if the user hands the device to another user, the aggregate score drops to 0 because the device is no longer being used by the user.

Challenges are able to be implemented to verify the user which will increase the aggregate score. For example, the user is requested to perform one or more tasks, and if the user's performance is verified, then the aggregate score is able to be increased to an amount above the threshold. For example, the user is requested to shake the device up and down four times, and based on the movement, the speed of the movement, any twists or twitches detected, the device is able to verify if the user is the correct user based on previous analysis of the user. Another example of a challenge involves having the user looking in various directions in front of the device's camera, where the system is able to compare the different poses with stored information or information based on the stored information. Similarly, the challenges are able to implement or incorporate Turing tests to prevent computer-based attacks/breaches.

After going through the analysis and/or challenge, if the aggregate score (e.g., a user's trust score) is above a threshold, then a transaction is authorized. As described herein, the transaction is able to be any transaction such as accessing the device, accessing a website, providing a payment/purchasing an item/service, and/or any other transaction. Different transactions are able to have the same or different thresholds. For example, simply going to a webpage may have a lower threshold than accessing a social media account which may have a lower threshold than authorizing a purchase of an item. The size of the amount/purchase (e.g., $5 vs. $50,000) is able to affect the threshold.

FIG. 20 illustrates a diagram of an aggregated trust framework according to some embodiments. The aggregated trust framework includes a mobile device 2000, one or more backend transaction servers 2002, and one or more dedicated cloud service devices 2004.

The mobile device 2000 includes a trust app configured to perform the analytics and challenges as described herein. The mobile device 2000 is able to include standard hardware or modified hardware (e.g., add-on sensors). The mobile device 2000 is able to be a mobile/smart phone, a smart watch, and/or any other mobile device. Depending on the implementation, results of the analytics and challenges are able to be stored on the mobile device 2000 and/or the one or more dedicated cloud service devices 2004. For example, the mobile device 2000 is able to include an app which performs the analytics and challenges including storing the results of the analytics and challenges, and then provides a transaction authentication (or denial) to the backend transaction servers 2002. In another example, the mobile device 2000 receives analytics queries and challenge requests from the dedicated cloud service devices 2004 and provides the information/results back to the dedicated cloud service devices 2004. The trust app is able to include or communicate with another device, to perform artificial intelligence and/or machine learning capabilities. The ID trust library is an SDK embedded inside the device (trust) app.

The backend transaction servers 2002 define discrete transactions, including a minimum trust score to perform each transaction. For example, the backend transaction servers 2002 communicate with a website server (e.g., social network, bank, online store) to gain access to the website (or other online service). The backend transaction servers 2002 communicate with the mobile device 2000 to receive a trust score (or other authorization signal), and if the trust score is above a threshold, then the transaction is able to be authorized by the backend transaction servers 2002. The transaction servers 2002 interact with an ID trust library, where the transaction servers 2002 provide policies to the ID trust library. In some embodiments, the ID trust library is stored within a device (trust) application. The ID trust library retrieves policies from the transaction server 2002, and then uses the policies and other criteria to generate a trust score. Each server transaction has different requirements for each transaction. As described herein, a task such as opening a bathroom door involves less security and identity confidence than opening a bank vault or entering a military resource. The transaction servers 2002 contain the policies and sends them to the device application. Then, the ID trust library processes a trust report. If the result complies with the given policy, the device app is allowed to perform the specific transaction.

The dedicated cloud service devices 2004 provide resources and services to clients (e.g., mobile devices). The dedicated cloud service devices 2004 include a trust analytics data feed, activity log feeds and phone security conditions. The dedicated cloud service devices 2004 are able to provide updates to the app on the mobile device 2000, communicate with the mobile device 2000 for a cloud-based implementation of the analytics and challenges, and/or for any other purposes.

In an exemplary implementation, a user attempts to perform a financial transaction with his online bank using his mobile device 2000. The online bank system communicates with the transaction servers 2002, where the online bank system waits for an authentication from the transaction servers 2002. The transaction servers 2002 verify that the user is who he says he is based on the mobile device 2000 determining a trust score for the user that is equal to or greater than the minimum trust score (e.g., threshold) for the transaction to be authorized. After the user generates a trust score that is above the threshold via the analytics and/or challenges, an authentication to perform the transaction is sent to the transaction servers 2002 which is able to provide the authentication information to the online banking system to perform the transaction. If the trust score is not above the threshold, then the transaction fails.

FIG. 21 illustrates a diagram of mobile trust framework functions according to some embodiments. As described herein, the mobile trust framework includes two major functions and the supporting framework.

In the step 2100, sensor data is received. Depending on the analytics and/or challenges, the sensor data is able to include movement data such as vibration detection by the sensors, and/or shaking movement, gait motion; input data such as swiping motions and/or keyboard/keypad input; voice/audio input; image/video input; and/or any other sensor/input data.

In the step 2102, trust analytics are implemented. The trust analytics software modules each run independently. In some embodiments, the modules are linked by graphical weighted decision tree algorithms, where multiple trust analytics trust scores are aggregated into a single trust score. The trust scores are dynamic and change from second to second, and are computed prior to any transaction. The trust analytics are able to include: traditional “know,” “have,” and “are” questions; dynamic biometrics including behavioral analysis; external behavioral factors such as location analysis; external factors such as environmental parameters; and/or device hardware/software behavioral analysis. Although a weighted decision tree is described herein, any structure (e.g., matrix) is able to be utilized.

In the step 2104, one or more challenges are implemented. Since each transaction performed has a minimum trust score, on the occasion where the current trust score is lower than the minimum, a challenge is used to prove the user to the mobile trust system. The challenges are able to be stored in a challenge stack, where a challenge module is algorithmically selected. Performing a challenge successfully raises the user trust score above the minimum threshold. Although a stack is described herein, any structure is able to be utilized.

In the step 2106, after the analytics and/or challenges, a resultant trust score is generated. The resultant trust score is used to determine if an authorization is provided. The authorization is able to be provided as a token, a certificate or any other authorization implementation. The authorization enables a transaction to occur.

In an exemplary implementation, a user initiates a transaction on a device app containing the ID trust library. The ID trust library connects to a transaction server and receives transaction policies and minimum trust thresholds. The ID trust library runs through the computational algorithms. The ID trust library computes the current ID trust score. If the resultant current trust score is below the threshold values, the ID trust library uses policies to select a challenge module, and the challenge module is executed, potentially raising the trust score. If the final trust score is above the threshold, the transaction is allowed to continue; otherwise, the transaction is not allowed.

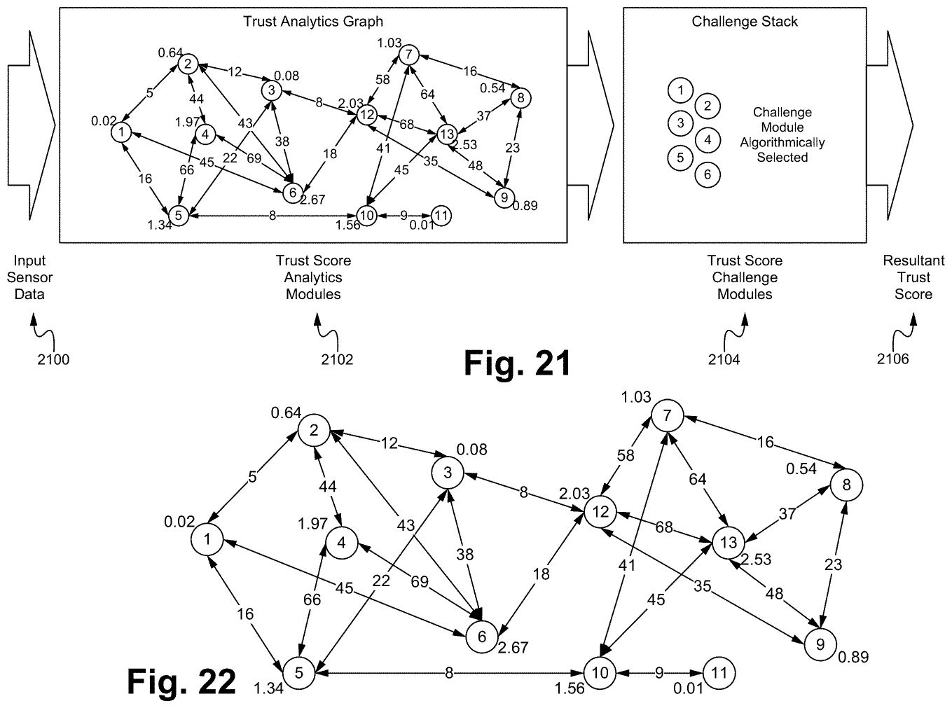

FIG. 22 illustrates a diagram of a weighted analytics graph according to some embodiments. The trust analytics are independent self-contained modules working together to construct a complex structure. The structure includes interrelated modules in a weighted decision tree graph. As more modules are added, the overall accuracy (or trust) increases. The analytics modules work together as a single system using technologies described herein.

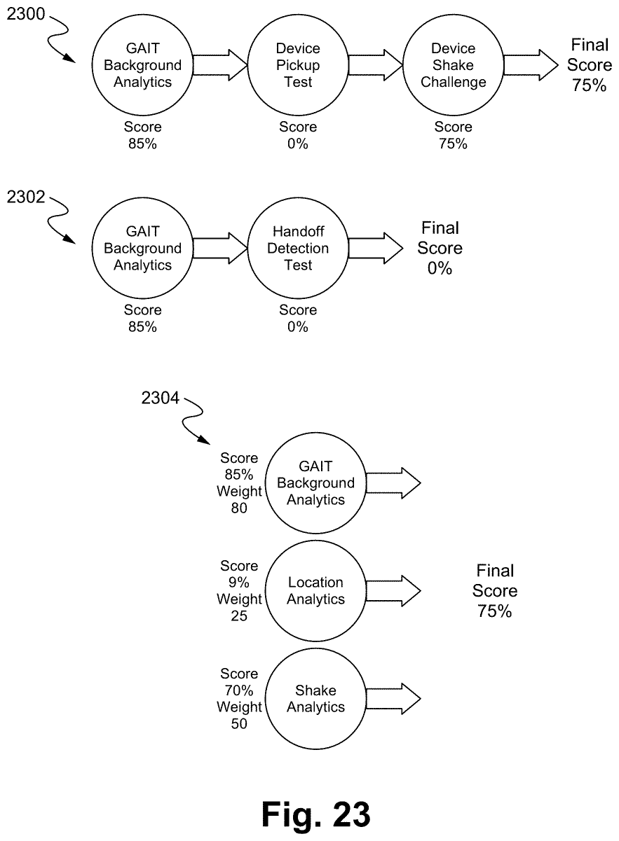

FIG. 23 illustrates diagrams of exemplary scenarios according to some embodiments. Depending on various contexts such as user behaviors, environmental conditions and other factors, the trust score analysis will navigate the decision tree graph with different paths. The analytics computation results are practically infinite.

In scenario 2300, a user's motion is collected in the background with a gait trust score computed continuously (e.g., 85%). Another analytics module with a higher weighting value can override the resulting trust score. In this scenario, a device pickup or device handoff test reduces the overall score drastically since the current user cannot now be verified. To verify the user identity, a challenge module is initiated (e.g., device shake challenge). Challenge modules are used if immediate user actions are desired, such as unlocking a door or logging into an Internet service.

In scenario 2302, after the gait background analytics, the handoff analytics module detected that the phone was handed to another user. This action drastically reduces the overall trust of the identity of the current user holding the phone.

In scenario 2304, tests are able to be run in parallel. Some types of analytics may operate independently at the same time. The combination of these modules can be combined, and using the priority weight values, an overall trust score can be computed. More complex scenarios using weights and other parameters used for decision branching are described herein.

Exemplary modules are able to be categorized such as: human movements, static image analysis, dynamic image analysis, voice print analysis, user location, external factors, device usage, and/or device internals. Human movements include a shake test, a gait test, micro-tremors, a pickup, and/or a handoff. Static image analysis includes facial recognition, ear shape, face with Turing test (e.g., user instructed to look up), and/or face with user ID (e.g., user face while holding up driver license). Dynamic image analysis includes continuous facial analysis and/or lip movement analysis. Voice print analysis includes continuous voice recognition and/or voice with a Turing test (e.g., the device instructs a user to say random words to thwart malware or recordings of the user's voice). User location includes movement vector analysis (e.g., user is on common routes), common locations (e.g., user is at home or work is more trusted than somewhere the user has never visited) and/or speed analysis (e.g., impossible travel scenarios). External factors include ambient light and/or altitude/temperature/barometric pressure. Device usage includes typing/swiping analysis, app usage analysis, and/or device login/startup. Device internals include device hardware anomalies and/or device software anomalies.

A trust challenge is a mechanism where the mobile trust system challenges the smartphone user to perform some predetermined action. This is used when the trust system cannot adequately determine the identity of the user. An example would be a user using the system to unlock an electronic lock. The user has an option to prove their identity and immediately open the door. When the user's current trust score is inadequate, a trust challenge is initiated. At the successful completion of the challenge, the user's trust score is increased adequately to open the door.

Turing tests in this context are used to guarantee the user identity is a human. Malware is an enormous threat today. User identities are commonly compromised by malicious software. Once a user's identity is exposed to malware, the user's identity can be used fraudulently. The trust challenge technologies use any of several biometric factors in combination with an action that can only be performed by a human. Examples of challenges with Turing tests include dynamic human interactions. Examples include: reading from the screen random words or pictures and saying them out loud. Generally, only a human can interpret the messages, and the human voice print identifies the specific user. Another example is identifying a video challenge. Another example is dynamic facial recognition of the user performing actions specified by the mobile technologies. Examples might be look right, look up, stick out your tongue, and more.

Exemplary challenge modules are able to be categorized such as: image analysis, human movements, voice prints, personal information, directed actions, and/or static biometrics. Image analysis includes a face with Turing test (e.g., facial recognition combined with instructions from the device), a face with User ID (e.g.,user's face and holding up driver license) and/or Facial 3D (e.g., user moves the device around his face). Human movements include a shake test. Voice prints include voice recognition and/or voice with Turing (e.g., user says random words instructed by the Trust framework. Personal information includes things the user knows such as mother's height, SSN, passwords/codes, date of special event, and/or many others. Directed actions include swipes, directed touch (e.g., touch areas or images on the screen), directed typing, drag objects, and/or pinch/spread. Static biometrics include fingerprints and/or image recognition.

### Software Bus

Inside the application is a software bus. Inside the software bus is a database, a computation engine, and a policy engine. The computation engine performs the calculations, and the policy engine includes the decision-making information. The computation engine includes a weighted scoring engine which involves a weighted matrix which is able to take a base score and additional scoring information from the multi-stage phases to generate an aggregated score.

The software bus connects to each phase (module) as described herein, and inside each phase (module) are pluggable components for each analytics element. For example, the software bus connects to the base module, the pre-transaction module, the external/environmental module, the device module, the hijack module, and/or the challenge module and/or the pluggable components within the modules. The pluggable components allow analytics elements to be added, removed or modified dynamically. The pluggable components are able to be programmed in an interpretive language.

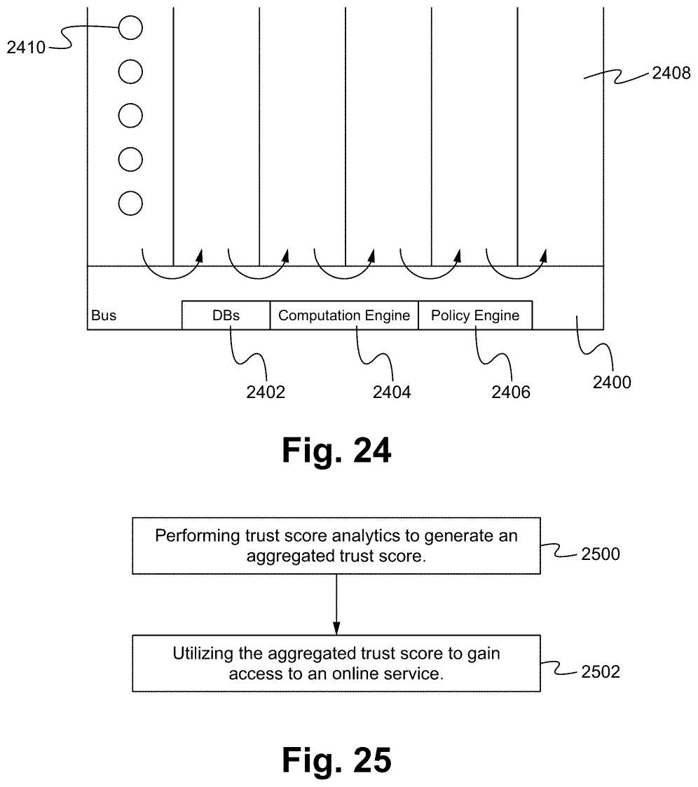

FIG. 24 illustrates a representative diagram of an aggregated trust system including a bus according to some embodiments. The aggregated trust system includes an application bus 2400 which enables modules 2408 (e.g., the base module, pre-transaction module, and so on) to communicate with each other. The application bus 2400 also enables pluggable components 2410 within the modules 2408 to communicate with pluggable components 2410 within other modules 2408. The bus 2400 includes a data structure 2402 (e.g., one or more databases), a computation engine 2404, and a policy engine 2406. The data structure 2402 is able to be used to store acquired information (e.g., from the sensors), calculated results (e.g., trust scores) and any other information. The computation engine 2404 performs the calculations, and the policy engine 2406 includes the decision-making information. The computation engine 2404 includes a weighted scoring engine which involves a weighted matrix which is able to take a base score and additional scoring information from the multi-stage phases to generate an aggregated score.

### User is the Password

Analytics that define a user are able to be used as a password for access to online transactions. As described herein, the analytics are able to include a user's physical attributes, gait, tremors/microtremors, face, ear, voice, behavior, vein patterns, heart beat, device usage, and/or others. The analytics generate a matrix of data, and each analytic is able to be broken down into components. For example, gait includes height, speed, walking, acceleration, gyroscope, and it follows a pattern match which is extrapolated into a pattern information structure. In another example, physical attributes are able to include a user's height, weight, skin color, hair color/style, birthmarks, scars, and/or other identifying physical attributes. Vein patterns are also able to be detected (e.g., using a mobile phone's camera to scan a user's face, arm or leg). Tremors or microtremors are able to be detected in a user's hand based on the accelerometer and/or other components in a mobile phone detecting very slight movements. Facial, ear or other body part recognition is able to be implemented using the camera of the mobile phone. Voice recognition is able to use the microphone of the mobile phone. In some embodiments, the voice recognition occurs without the user specifically focused on passing a voice recognition test. For example, the mobile phone “listens” to nearby voices including detecting the user's voice. The mobile phone is also able to “listen” to the user's voice while the user is talking to another person to analyze the voice and determine if the voice is that of the user. Other behavioral analysis is able to be performed as described herein such as analyzing the locations that the user and the mobile phone go to, how long they are there, which web sites are visited, and/or any other behaviors/actions that the user takes that are repeated and recognizable. Using the mobile phone, a microphone or another sensor of the mobile phone is able to detect a user's heartbeat. For example, the mobile phone is able to be placed against a user's body or a sensor is connected from the mobile phone to the user's body, and the mobile phone is able to detect a user's heartbeat including any specific, unique heart rhythm. In some embodiments, all of the analytics patterns are aggregated into a pattern matrix. The pattern matrix is a multi-variant matrix which is able to account for changes in one or more of the analytics patterns. For example, if a user has a broken nose, his detected face pattern may be off when compared with the stored face pattern information, so the other analytics or additional analytics are used to compensate to ensure the proper user is able to perform transactions while also ensuring that an improper user is blocked from performing transactions. The stored data is continuously, dynamically changing to account for changes in the user (e.g., a user's voice changing, a user's hair changing, and many others). The stored data is able to use artificial intelligence and machine learning to maintain a knowledge base of a user and many possible attributes. For example, not only is the user's normal gait learned and stored, but if the user has a slightly different gait after exercising, and a very different gait when injured, the various gaits are able to be learned and stored, so that the gait analytics are able to be used regardless of the user's current state.

FIG. 25 illustrates a flowchart of a method of using the user as a password according to some embodiments. In the step 2500, trust score analytics are performed to generate an aggregated trust score. As described herein, the trust score analytics utilize sensors and other devices to acquire information about a user to determine if the device is being used by the expected/appropriate user (e.g., owner of the device). The analytics include base information, pre-transaction information, external/environmental information, device information, hijack information, and/or challenge information. In some embodiments, a token or a hash is generated using the trust score analytics. In some embodiments, the token is a Non-Fungible Token (NFT). The token is able to be a user's password, facial scan or other acquired data and/or used as a password or otherwise to gain access to a service (e.g., an online service such as Facebook or a bank account). In some embodiments, the NFT is a unit of data stored on a digital ledger, referred to as a blockchain, that certifies a digital asset to be unique and not interchangeable. The token is able to be generated in any manner, for example, if a user's trust score is above a threshold, then a token is generated to represent that user. In some embodiments, each time a user's identity is confirmed using the trust score analysis, a new token is generated, and the old token is deleted and/or made unusable. The token and/or the trust score are able to continuously evolve as more data is acquired about the user. The token is able to be stored locally and/or remotely. In some embodiments, a private token or certificate and a public token or certificate are used such that the private token is stored locally and the public token is able to be shared, where the public token is used to gain access to a service. For example, the public token merely includes general information that indicates that User A is actually User A; however, the private token includes the specific information such as stored biometric (human characteristic) information and other personal information that has been acquired, tracked and/or analyzed. The public token is able to be based on or linked to the private token. For example, if the private token becomes invalid for some reason, then the public token also becomes invalid. Any public-private key exchange is able to be utilized based on the human characteristic information acquired. A homomorphic data vault is able to be used to maintain data securely, where the data vault is able to be interrogated for information (e.g., queried do you contain this?), but the actually data is not accessible by an external source.

In the step 2502, the aggregated trust score is utilized to gain access to an online service. For example, a mobile device is used to log in to an online service, and if the aggregated trust score is above a threshold, then the mobile device sends an authentication certificate or other information to access the online service (e.g., social network login). If the aggregated trust score is not above the threshold, then the mobile device does not send the authentication certificate or other information. If the aggregated trust score is not above the threshold, then the user is able to be challenged (e.g., prompted to perform a challenge action). Based on the challenge the user may raise their trust score above the threshold (or not), and if the trust score is above the threshold, the authentication certificate is able to be sent to access the online service. The access is not limited to online services. Any access (e.g., open the front door) is able to be implemented using the aggregated trust system including the user is the password aspects. In some embodiments, fewer or additional steps are implemented. For example, if an aggregated trust score is below a threshold, then one or more challenges are provided to affect the aggregated trust score. In some embodiments, the order of the steps is modified. The authorization is able to be used as a password to access any system. For example, the password is able to be used to access the mobile device, web pages/social networking pages, secure devices, online services, and/or any other device/system/service that utilizes a password to gain access. In another example, a user navigates using a web browser to a web page which requires a password or other authentication to access the web page. Instead of providing a password which is able to be stolen or hacked, the user's mobile device authenticates the user based on the aggregated analytics described herein and provides an authentication certificate or other implementation to indicate to the web page that the user is who he says he is (e.g., the accurate user). As described above, a generated token is able to be used as a password to gain access to a service. For example, the user is able to provide the previously generated token to the service which verifies the user as the user. In another example, the service automatically analyzes the token and verifies the user based on the token. In yet another example, a public-private key exchange is implemented with the token generated from the human characteristic information.

### Architectural Overview

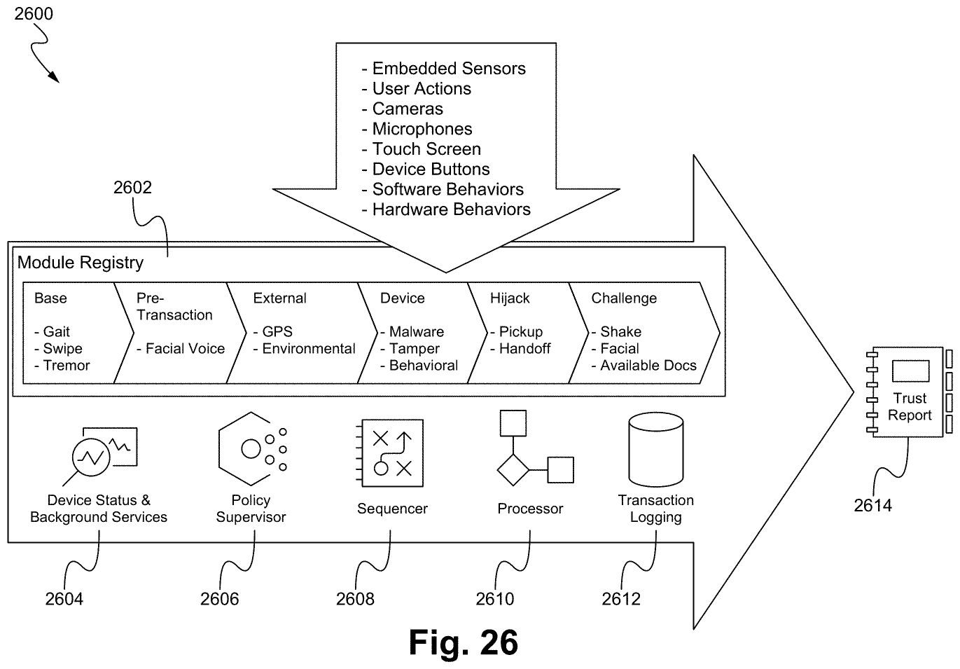

FIG. 26 illustrates a diagram of an architectural overview of the ID trust library according to some embodiments. The ID trust library 2600 includes a module registry 2602, device status and background services 2604, a policy supervisor 2606, a sequencer 2608, a processor 2610 and transaction logging 2612. The ID trust library 2600 is able to be used to generate a trust report 2614. As described herein the module registry 2602 includes a base module, a pre-transaction module, an external module, a device module, a hijack module and a challenge module. The module registry 2602 utilizes embedded sensors, user actions, cameras, microphones, touch screen, device buttons, software behaviors, and hardware behaviors to perform identification analysis.

Data is collected about the user, the device and external conditions. Each of the ID trust modules is responsible for the collection and processing for their respective functions. Modules are grouped by classes and processed in stages. Collected data is stored in the device's local storage or on a remote server. In each stage, the analytics are processed by a rules engine. The intermediate trust scores for each stage are processed using a graphical decision tree algorithm and produce a final score. The history of all transactions and score are able to be analyzed to produce a trust report 2614.

### ID Trust Module

In some embodiments, the modules serve a single purpose. A module is isolated from all other modules. A module can only perform its designed action to generate its results. It does not communicate with any other module nor does it access any other part of the ID trust environment. Modules conduct their intended ID analysis or challenge upon request, then return its result. The output is two pieces of data: 1) a resulting score and 2) a confidence level of that score.

A module may perform its action on demand, or it may be given background time to collect data. The module can maintain a history and then produce the resulting score based on that history.

A score is the result of the module's security action. Values are within the range of 0-100. Confidence is a high, medium, or low level of the quality or reliability of the resulting score. For example, if there is a test that normally takes several iterations to complete, and if that number of iterations was done, then the resulting score could be given a high level of confidence. But if the challenge was only completed once or done quickly, then it would have a low level of confidence.

### Module Class

There are six different classes of ID trust modules. Each module is defined to be of only one class. Each class conducts a certain type of challenge described below. There are two types of modules. An analytic module performs its function without user interaction. The other is the challenge module, which interacts with the user. Some modules may run in the background. Others can only execute on-demand and may involve user interaction. Examples of analytics modules that may run in the background include gait (a person's pattern of walking), device usage like typing patterns, micro-tremors generated by the users, and others.

As described herein, the base class is a group of modules are executed to perform a base set of analytics, continuously monitoring the behaviors of the user. This produces a near real-time continuous score. Behaviors which are consistent with the historical behaviors of the user are analyzed. Consistent behaviors may be extremely accurate and can identify a user with fingerprint accuracy.

The pre-transaction class is a group of analytic modules which are executed to identify the user performing the transaction. An example would be to have the camera “look” at the person holding the phone at the time of the transaction. This would provide a sanity check and is possibly only performed if the base trust score is low, and the system is suspicious.

The external class is a group of analytics that performs tests of external factors such as GPS, common routes, barometric pressures, altitudes, time/date, ambient light and others. The module is only used in certain scenarios. Examples include: financial transaction but a user is outside his normal location; or unlocking a door, but the user's GPS location is not near the door. The module will commonly test for suspicious conditions such as: impossible travel—for example, the GPS location history shows the user was in Europe, but 5 minutes later another transaction is performed in Borneo. Suspicious location—for example, the transaction is to unlock a door, but the phone GPS is nowhere near the door. Unusual locations—for example, the user performs a transaction and is not home, at work, or at a common location. For critical transactions, if the user is somewhere unusual, the transaction will involve a higher trust score threshold.

The device class is a group of analytics that tests for the health or security condition of the device itself. These modules analyze the condition of the device hardware and/or operating environment. Any detection of suspicious device health or functionality will drastically reduce the current trust score. These tests are monitoring for conditions such as: hardware tampering, the device has been spoofed by another device, or the device operating system has potential malware.

The hijack class is a group of analytics which monitors for conditions where the device is not in position of the registered user. Any form of hijack detection will drastically lower the current trust score. Examples of hijacks include: pickup detection—the device was set down, then picked up. The device may have been picked up by the owner, but this could be anyone; or handoff detection—the device monitors for when the phone is handed from one person to another. Once this condition is detected, the person holding the phone is suspect, and the trust score is reduced drastically.

A challenge module interacts directly with the user and challenges the user with some action which: tries to guarantee the transaction being performed is done by a human and not some form of malicious software. Malicious software examples are bots, viruses or trojans. Old fashioned versions of this type of challenge include requesting personal information about the user, such as “mother's maiden name.” Due to the amount of personal information having been stolen and shared by bad actors, such challenges are no longer secure. Biometric versions of this challenge include having the user identify themselves by the hardware fingerprint detector. These challenge modules request users to perform actions and can be a nuisance and are only called upon as a last resort when the analytics cannot appropriately identify the current user.

### Sequencer

ID trust modules are chosen by a defined order determined by their class. Once the set of modules has been chosen, they are called to perform their challenge. The first module is called, and its result is stored. Then the next module is called, the result is stored, and so on until the last stage has completed.

The sequencer 2608 performs the following: building the proper chain of modules to calculate the trust score receiving policies from the transaction server for each given transaction, and calling modules that involve periodic execution time for monitoring activity in the background. An exemplary sequence of the modules implemented by the sequencer 2608 is Base→Pre-transaction→External→Device→Hijack→Challenge.

The sequencer 2608 determines which module classes are used based on the following criteria: which module class to choose is based on the given policy. The policy is given to the sequencer 2608, which then determines the class of modules to produce the ID trust score. The determination of which class to use for a given policy is complex. If there is more than one module within the chosen class, then module priority is used in the selection of a module. In the case where there are multiple modules selected at the same priority, the resultant trust scores are combined mathematically into a single score. Module priority values are high, med, or low. The value is determined by the security admin user within the admin console of the transaction server.

Once the classes are chosen, constructing the sequence of modules is relatively simple.
1. Select the modules with the highest priority within its class for the specific stage.
2. Add the next module to meet the policy criteria.
3. Repeat until the last module has been added.

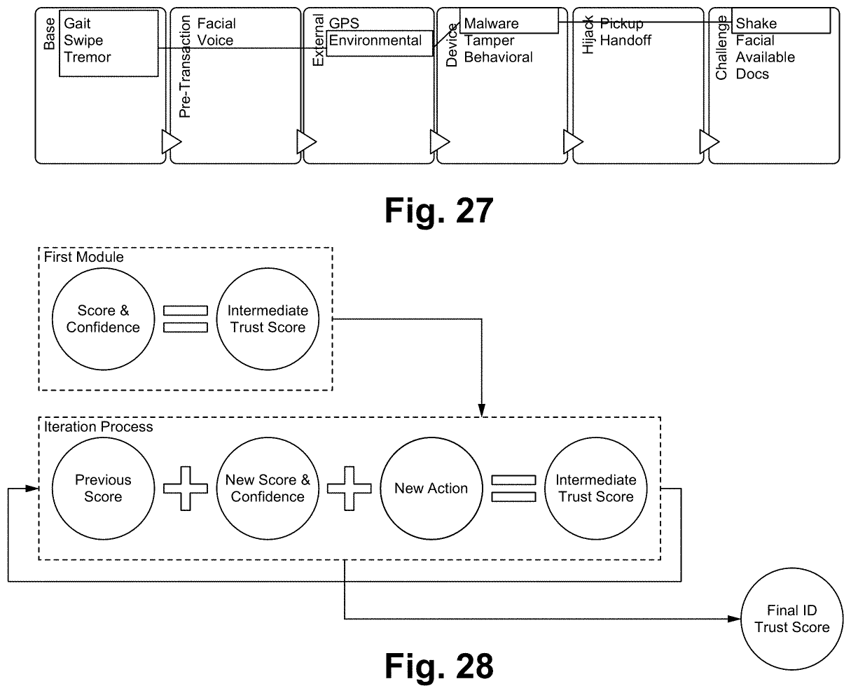

FIG. 27 illustrates a selection of modules chosen for a given policy according to some embodiments. In the example, gait, swipe and tremor are selected from the base class, followed by environmental factors from the external class, then malware from the device class, and finally a shake challenge.

### Processor

The sequencer calls each of the chosen modules and stores their results (score and confidence). It is the processor 2610 that evaluates all of the stored results to determine the final ID trust score. The processor 2610 logs the details used to process the trust score.

There are two key attributes used by the processor 2610: module score and confidence. These two results are provided by the module. Confidence values are high, med, or low and determine if the module's result should affect the computation. Module action—the action to perform is defined by the class of module. The base class establishes a base score and has no action. The other classes have the action to raise or lower the score. Modules produce an intermediate score, and their results are processed in a specific sequence. For example, a base module's result can be overridden by a later hijack module. There are currently six classes of modules, one for each stage. This process performs combined computations and algorithms to derive a final trust score. The following defines the action to perform on the given result of the ID trust module based on its class. The base class generates a base score, a pre-transaction raises the score, and the external, device, hijack classes lower the score. The challenge class is used to raise the score.

The steps below outline the process for obtaining the final ID trust score. FIG. 28 illustrates the logical flow according to some embodiments.
1. First module's results are obtained from the storage and saved as an intermediate result.
2. Next module's results are obtained from the storage.
3. Results of the first intermediate result and the new result are compared according to their confidence and action.
4. The intermediate result may be kept or replaced by the new result.
5. Repeat the process until the last module's results are computed.

### Policy Supervisor

The policy supervisor 2606 controls all the logic, calling on the sequencer 2608 and processor 2610 to perform their tasks. Each transaction has different policies including a minimum trust score. If the policy requirements are not met, the transaction is blocked until the trust score increases to an acceptable level (e.g., above a threshold). The policies are defined at the transaction server.

This logic happens during each transaction and does not impact the user experience.
1. Obtain the policy from the transaction server.
2. Call the sequencer to build the chain of modules for the given policy.
3. Pass the chain of modules to the processor.
4. Compare the final ID trust score with the policy.
5. If the score is above the threshold, then the process is complete.
6. If the score is below the threshold, then repeat steps 2-4 adding a challenge module to force a user interaction.

A policy is a set of criteria provided by the transaction server. The server sends the policy to the ID trust library during a transaction. The policy supervisor obtains the trust score based on that criteria. The following are some of the criteria: transaction description, transaction minimum score threshold: minimal acceptable score, location: use the device's location, transaction code, transaction weight, factor priorities, routes, speed, ambient light, temperature/humidity/barometric and the challenge array.

### Modules Registry

Each module is registered when added to the ID trust library. Adding the module to the ID trust library is simple by including the module at build time by static linking the module inside the SDK project. Registering the module to the ID trust library is accomplished by inserting a record in the module database in the modules registry. The fields in the modules registry include: module name, module description, module class, module priority (determined by the transaction server), and background (activity to perform and rule when to be called).

### Logging

Logging is a function performed by the processor. As the processor obtains each of the module's results, it logs the module's name and results. The processor also logs the intermediate results as it is processing the chain of all modules.

The system keeps records of all transactions and the results used to calculate all trust scores such as: the analytic descriptive fields, analytic resultant input fields, data storage, policies storage, and default server policies.

### Security

The SDK is able to be compiled and distributed in binary for security and competitive reasons. Malicious people should not be able to view the software sources and thereby be allowed to inspect and detect security vulnerabilities.

### API Specifications

The specific API definitions are documented in a separate ID trust library technical specification: ID trust library for client and server, ID trust module API, the APIs listed map to software methods and exposed to host app environments, and this product feature is written in a language such as C/C++ which is used in common with many host environments.

### Packaging and Delivery

The app and/or system is packaged as an SDK, which includes the ID trust functionality, the analytic modules, the challenge modules and adapter modules to support the host environments.

### Compatibility

The SDK is available for client apps on iOS and Android devices, in their native format. SDK is available for servers in Node.

### User Interface & User Experience

The GUI framework is developed as part of the resulting analytics and challenge modules. These GUI templates are skinned to match the host app appearances. If the user is asked to perform a task, such as shake the device, instructions are simple and clear. Only a few words and ideally images are used to explain how to perform the task.

### Performance

Prior to each transaction, the analytics system performs the trust score analysis. When the trust score is inadequate, the transaction is blocked. This operation is completed quickly to maintain a good user experience.

Any delay in the trust analysis degrades the user experience, so this system performs at sub-second levels. This performance includes strategies such as caching, performing analysis in background processes, using a central database to aggregate analytic module resultant values, and others.

The exception is if the user is asked to perform a task, such as shaking the device. That obviously interrupts the authentication process.

### Multi-Stage Scoring

The following examples show processing for each stage, producing a final resultant score.

| Stage | Module | Action | Module Score | Intermediate Score |
|---|---|---|---|---|
| 1 | Base | Base | 80 | 80 |
| 2 | Pre-Transaction | Raise | 90 | 90 |
| 3 | Environmental | Lower | 80 | 80 |
| 4 | Device | Lower | 60 | 60 |
| 5 | Hijack | Lower | 0 | 0 |
| 6 | Challenge | Raise | 80 | 80 |

Resultant Score 80

Good Base Score, Pickup/Handoff detected

In this example, the phone monitoring the user behavior with the base modules, but one of the hijack modules detected suspicious behavior and reduced the trust score to 0. This causes a challenge to be performed to raise the trust score to an acceptable level.

| Stage | Module | Action | Module Score | Intermediate Score |
|---|---|---|---|---|
| 1 | Base | Base | 80 | 80 |
| 5 | Hijack | Lower | 0 | 0 |
| 6 | Challenge | Raise | 70 | 70 |

Resultant Score 70

### Good Base Score, Device Tampering Detected

In this example, the phone monitoring the user behavior with the Base modules, but one of the Device modules detected suspicious behavior or tampering and reduced the trust score to 60.

### Good Base Score, Device Tampering Detected

| Stage | Module | Action | Module Score | Intermediate Score |
|---|---|---|---|---|
| 1 | Base | Base | 80 | 80 |
| 4 | Device | Lower | 60 | 60 |

Resultant Score 60

### Good Base Score, Suspicious Environmental Conditions

In this example, the phone monitoring the user behavior with the base modules, but one of the environmental modules detected a condition which the specific transaction has specific environmental requirements.

| Stage | Module | Action | Module Score | Intermediate Score |
|---|---|---|---|---|
| 1 | Base | Base | 80 | 80 |
| 3 | Environmental | Lower | 30 | 30 |

Resultant Score 30

This specific transaction had specific location requirements. The environmental module common locations detected that the user/device was located where the user has never been detected and reduced the trust score, subtracting 50 points.

As described herein, analytics are able to be used to identify a user of a device. Examples of analytics include tremor, gait, vehicle motion, and facial recognition. The analytics are able to be grouped into related and unrelated analytics. For example, tremor, gait and car motion are able to be considered related analytics, and they are unrelated to facial recognition. The determination of related and unrelated is able to be performed in any manner. For example, if the analytics share common elements such as being related to motion or being determined using an accelerometer, then they are related. By using related analytics, analysis and feedback are able to be shared among the analytics modules to improve machine learning for user identification.

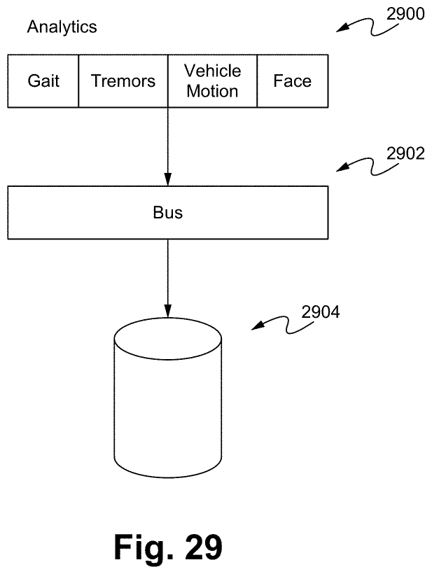

FIG. 29 illustrates a diagram of analytics with shared traits according to some embodiments. The analytics 2900 include tremor, gait, vehicle motion, facial recognition, and many others described herein. Each of the analytics 2900 is trained. In some embodiments, the training of the analytics 2900 only occurs when the confidence that the current user is the authorized user is high or very high (e.g., confidence of the user is above a threshold such as 95% or 99%). The training involves detecting user activity/features (e.g., motion) and providing feedback as to whether the detected activity/features are true/correct or false/incorrect. Instead of the training and feedback applying to a single analytic, the training and feedback are able to apply to related/grouped analytics. For example, analytics that involve motion or the use of the accelerometer to detect motion are able to be considered related analytics; whereas, facial recognition uses a camera to scan a user's face. The related analytics are able to be trained simultaneously because they have shared traits. For example, as a user is walking with a mobile device in his hand, microtremors are able to be detected/analyzed for the tremor/microtremor analytics, and the user's gait is able to be detected/analyzed for the gait analytics. The detection/analysis is able to be used for machine learning of the analytics. In another example, while a user is walking with a mobile device in his hand, the gait is able to be detected/analyzed, and the user's hand motions are able to be detected/analyzed, where the same information is received but used for two separate analytics (gait, hand motion) since the analytics share a trait. In some embodiments, the analytics share a single trait, and in some embodiments, multiple traits are shared.

As the analytics receive and analyze user information, the received information, any appropriate analysis information such as links to classes, and any other learning information is sent to a bus 2902 to direct the information to be stored in a data store 2904. The stored data and learned information are used by the analytics to determine whether a current user of the device is the correct, authorized user (e.g., owner).

Training, feedback and data filtering are able to be performed and received for each of the analytics (including multiple analytics simultaneously). For example, if a user is riding in a car, the vehicle motion analytics are able to detect/analyze the situation, but also, the mobile device may detect tremors/microtremors. However, these tremors/microtremors may be from the vehicle and/or at least change the detected tremors when compared to a user simply standing an holding a mobile device. Therefore, the situational information (e.g., feedback from the vehicle motion analytics) is able to be communicated to the tremor analytics, so that the acquired information is processed correctly (e.g., ignored while in a vehicle, classified as tremor while in a vehicle versus tremor when not in a vehicle, or adjusted based on the vehicle vibrations). In another example, the gait and tremor analytics share information (e.g., feedback). Furthering the example, a user's heartbeat is typically different when he his calmly standing still versus when he is walking, and the user's heartbeat could affect the microtremors detected, so the gait analytics is able to share the information that the user is walking and/or that the user's heartbeat is elevated, so that the microtremor analytics module is able to account for the fact that the user is walking (e.g., the microtremor analytics module distinguishes/classifies data based on the other actions the user is taking at the time such as walking versus sitting versus standing). It is also important to filter out extraneous information that could cause improper learning. For example, if a user is on an escalator, is running a marathon, or dropped his phone, all of these external vibrations are able to confuse the device and lead to poor input data and incorrect analysis by the analytics. Therefore, the analytics are able to use the shared information to better determine what is going on with the user and whether the information is valid, correct and useful data to acquire and use for learning. In some embodiments, if the data is determined to be corrupted in that there are extraneous factors that are affecting the data such that it is not useful for learning, then the acquired data is ignored/deleted. In some embodiments, the data is classified/grouped in a manner such that a first set of data under a first set of circumstances does not affect a second set of data under a second set of circumstances. For example, if a user is a marathon runner, then acquiring the user's tremor information while the user is running is still useful information (potentially many hours per week running), but it will likely be different than the user's tremor information while at rest.

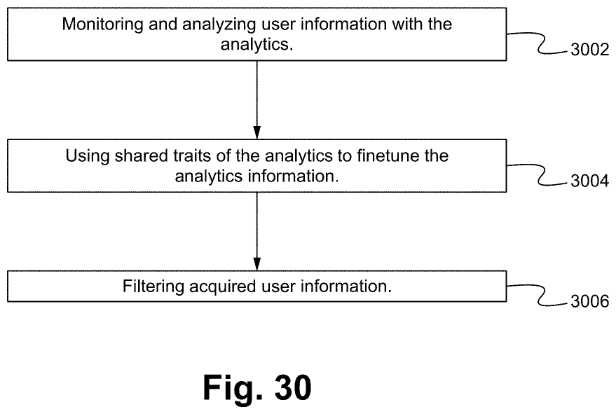

FIG. 30 illustrates a flowchart of a method of implementing analytics with shared traits according to some embodiments. In the step 3000, a user activates analytics tracking on a mobile device. For example, a user activates a new phone, and verifies that the user is the correct owner of the mobile device. The user does not necessarily perform activation of the analytics tracking; rather, in some implementations, simply activating a new phone causes the analytics to be activated. In some embodiments, the analytics are part of a background application which is part of or separate from an operating system and automatically runs.

In the step 3002, when a mobile device is sure (e.g., confidence above a threshold) that the user is the correct user (e.g., owner of the device), the analytics monitor and analyze user information such as user actions, other user features (e.g., face, voice), and/or any other user identification information. As described herein, examples of analytics include gait, tremors/microtremors, vehicle motion and facial recognition. The analysis of the user information includes specific details related to the user such as speed of gait, patterns of microtremors, driving patterns, identifying facial features, and much more. The information is stored to be later compared for user identification purposes. Some of the details are shared between the analytics modules, so the gait of a user and/or vehicle motion may affect the microtremors.

In the step 3004, the shared traits are used to fine-tune the analytics information. The shared traits allow information among related analytics to be shared among the related analytics. Additionally, feedback from each of the analytics is able to be shared among the related analytics. For example, if a user is walking with a device, the gait information is able to be shared with the microtremors analytics, so that the microtremors analytics are able to recognize that the microtremors are occurring while the user is walking. As discussed herein, the microtremors of the user at rest are likely to be different than microtremors when a user is walking which are likely to be different than microtremors when a user is running. The information acquired is able to be classified differently or other actions are able to be taken such as discarding/filtering the information. The fine-tuned data is stored appropriately such as corresponding to each related analytics module, and in some embodiments, in classifications or sub-classifications for each related analytics module. For example, in the microtremors analytics module, there are classifications of at rest, walking, running, and driving, each of which store different information based on the actions that the user is taking.

In the step 3006, acquired user information is filtered while the user utilizes the mobile device. Filtering the user information is able to be performed in multiple ways. For example, if the user information is acquired while external forces corrupt the acquired user information, then the user information is discarded. For example, if a user is talking into his phone, and a friend yells into the phone, then the voice acquired would be a mix of the user's voice and the friend's voice, which is not useful for voice recognition, so the data is not used for machine learning and is discarded. Determining whether to discard information is able to be implemented in any manner such as analyzing the acquired information and comparing it to the currently stored information, and if the difference between the information is above a threshold, then the acquired information is discarded. In another example, a user is queried about the difference (e.g., is your new gait because of an injury), and depending on the user's answer, the acquired information may be discarded. In another example, if feedback from a related analytic indicates that the acquired information is unreliable (e.g., it is determined the user is in a vehicle based on GPS feedback), then the acquired information is discarded (e.g., the microtremors from the vehicle corrupt the user's hand microtremors). The user information is also able to be filtered into classifications based on the shared details of the analytics and the feedback from the analytics. When the shared details from one analytics module affects the data of another analytics module, the data is able to be classified separately from previously stored analytics information.

The acquired user information is used to continuously improve learning about the user for the purposes of user identification. An important aspect of learning is that the correct data is used. Therefore, by filtering acquired information that is corrupt, incorrect or otherwise unhelpful, the learning process is more efficient and more accurate such that the device is able to more accurately and more confidently identify the user.

In some embodiments, the order of the steps is modified. In some embodiments, fewer or additional steps are implemented.

In an exemplary implementation, after a user purchases and activates his new mobile phone, a 5 minute identification period is implemented, where a user is directed to perform tasks such as holding the phone, walking while holding the phone, taking a scan/image of the user's face, ear, other identifying feature, talking for voice recognition, typing using the keypad, and/or perform any other identifying steps. After the identification period, the mobile device continues to monitor the user with the analytics. In some embodiments, to ensure that newly acquired data after the identification period is still for the correct user of the device, the user performs an authentication procedure as described herein (e.g., performing known tasks, facial recognition, biometric scans, and/or answering a challenge). Depending on what the user is doing, the analytics will continue to learn and store additional information, possibly generate new analytics classifications or subclassifications, and/or ignore/delete/filter acquired information that is determined to be unhelpful in learning. For example, during the initial identification period, the user walked while holding the phone, but did not run, so based on the accelerometer, GPS and/or other location/tracking devices/applications in the phone, if it is determined the user is running, the microtremors while the user is running are also able to be detected and stored in a new classification under microtremors related to “while running.” In another example, the user is mountain biking (as determined using the accelerometer, GPS and/or other location/tracking devices/applications) which causes irregular tremors which are unhelpful in learning about the user regarding microtremors in the user's hand, so this acquired information is discarded. The analytics with shared details are able to enable a device to continuously learn useful information about a user which is able to be used to properly identify the user while also avoiding learning misleading or erroneous information which may cause a misidentification of the user.

The analytics with shared traits are able to be implemented on a user device and/or a server device. For example, a mobile phone is able to include an application with analytics modules with shared traits to implement learning based on a user's actions and features. In another example, a server device receives information from a user's mobile phone, and the analytics with shared traits on the server device are able to be used to perform learning based on the received information of the user's actions and features.

A shake challenge is able to be used for identification purposes. The shake challenge involves a user device directing a user to shake the user device a specified number of times, and based on the internal components/mechanisms of the user device, the user device is able to identify the user as the user shakes the user device.

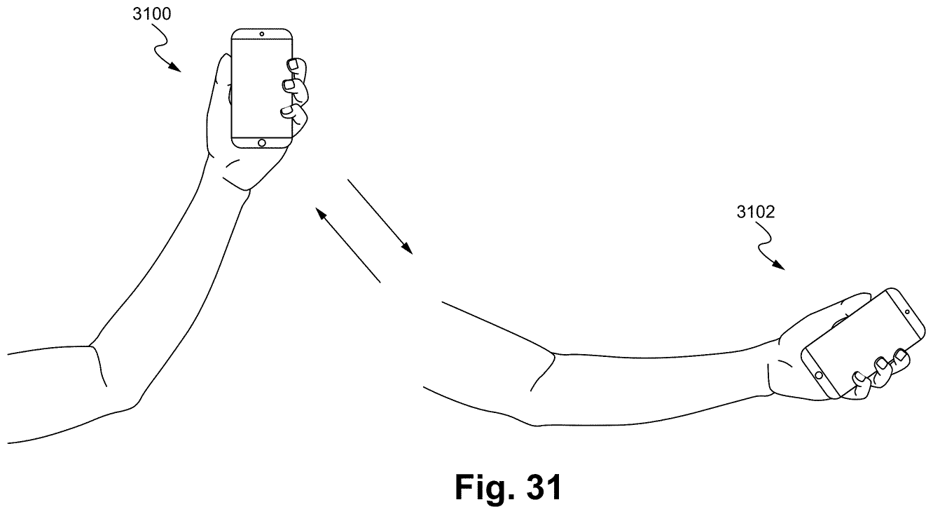

FIG. 31 illustrates a diagram of a user shaking a user device according to some embodiments. As described herein, a user is asked a challenge question or another challenge implementation if the user's trust score (or other score) is below a threshold. To prevent a malware attack, an application on the user device asks the user to shake the device (e.g., via text on the screen or an audible question). In some embodiments, a randomization element is involved in the request such as the number of times to shake the device, a specific direction to shake the device, a timed pause between each shake, and/or any other randomization such that a malicious program is not able to record a previous capture of a user's shake and trick the user device (e.g., spoofing).

When the user performs the shake, the user holds the device in his hand, and shakes the device in the manner specified (e.g., shake the device 3 times). The user device includes components such as accelerometers, gyroscopes, manometers, cameras, touch sensors, and/or other devices which are able to be used to acquire specific movement information related to the shake. For example, the components are able to detect aspects of the shake such as how hard the shake is, the speed of the shake, the direction of the shake, the rotation of the device during the shake, microtremors during the shake, where the user holds the device, and/or any other aspects of a shake. A camera of the device is able to scan the user while the shake occurs to provide an additional layer of analysis. Typically, a user shakes a device in a similar manner (or possibly several similar manners). After many shakes of the user device, the aspects and patterns are able to be detected such that a user's shake is similar to the user's fingerprint in that it is relatively unique. Although any movement is able to be implemented in accordance with the description herein, a shake involves a user moving a user device up and down, and/or forward and backward. The motion typically involves bending movements from a user's wrist, a user's elbow and/or a user's shoulder. For example, in position 3100, the user device is in an up position, and in position 3102, the user device is in a down position, and the shake movement goes from position 3100 to position 3102. In some embodiments, a full shake involves an added step of going back to position 3100.

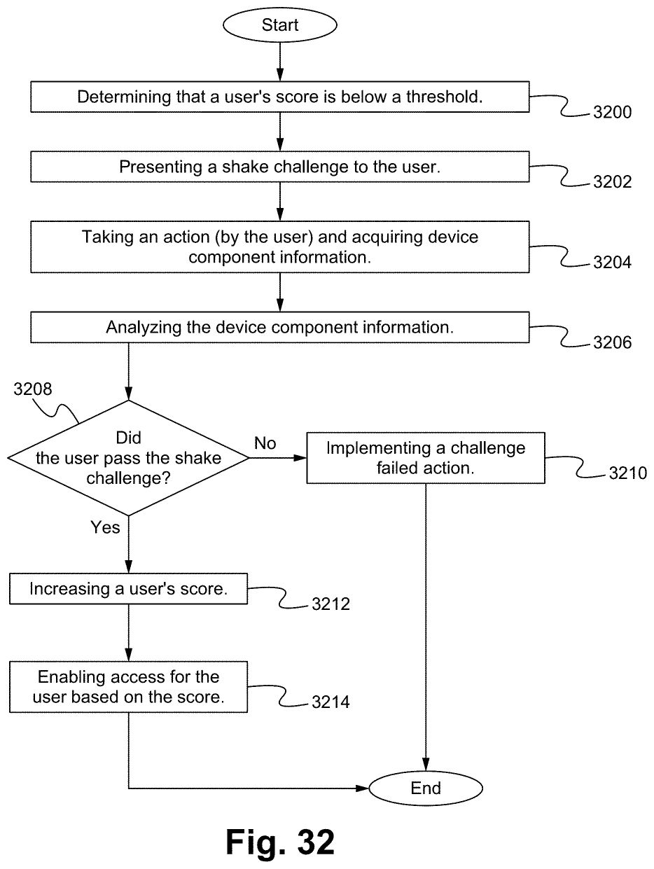

FIG. 32 illustrates a flowchart of a method of implementing a shake challenge according to some embodiments. In the step 3200, it is determined that a user's trust score (or other score) is below a threshold. For example, after a user puts his mobile phone down, and then picks up the phone, the phone is not sure that the user is actually the authorized user, so the user's trust score is below a threshold. In some embodiments, a shake challenge is implemented regardless of a user's trust score (e.g., for initial training of the device).

In the step 3202, a shake challenge is presented to the user. Other challenges are able to be presented to the user as well. Presenting the shake challenge is able to include sub-steps. A randomized aspect of the shake challenge is determined. For example, any of the following are able to be determined at random (e.g., using a random number generator): the number of times to shake the device, how a user is instructed to shake the device (e.g., audibly, via a text message), and/or any other specific details related to the shake challenge (such as the direction of the shake or a pause duration between shakes). The user is then instructed to shake the device the determined number of times. For example, a mobile device plays an audible message for the user to shake the device 3 times. In another example, a video is displayed visibly showing the number of times to shake the device.

In the step 3204, after the user has been instructed to perform the shake challenge, the user takes the actions as directed. For example, the user shakes the device 3 times. While the user shakes the device, the device utilizes components to detect and measure aspects of the shake. The components include accelerometers, gyroscopes, manometers, cameras, touch sensors, and/or other devices (and associated/corresponding applications) which are able to be used to acquire movement information related to the shake. For example, as the user shakes the device, the accelerometers and gyroscopes detect the speed of the shake, the direction/angle of the shake (e.g., straight up and down, side to side, the specific angle), the rapidity of the stop/change of direction, if there is any twisting of the device while being shaken and so on. Microtremors and rotations of the device are able to be detected as well. The manometers and touch sensors are able to be used to detect how hard the user grips the device while shaking, and the specific pressure points where the user grips the device. For example, some users may grip the device with two fingers, one in the front of the device and one in the back of the device. In another example, some users grip the device by placing four fingers on one edge of the device and a thumb on the opposite edge of the device. Some users have a very tight/hard grip, while other users have a weak/loose grip. Users are able to grip the device in any manner, and the device is able to determine the exact location of the fingers, the pressure of each finger, and any other details of the grip. In some embodiments, a camera of the device is able to scan the user (or another object) while the shake occurs to provide an additional layer of analysis. For example, the user is directed to hold the device such that the camera faces the user, so the device is able to perform facial/body recognition during the shake to provide an added layer of security. The components and the information acquired from the components are able to be used to determine the number of shakes. For example, based on acceleration, speed, direction and/or any other information acquired using the components, each motion by the user is able to be determined and how often that motion occurs is able to be determined. Furthering the example, when the user has not started shaking, the speed recorded by the accelerometers is roughly 0; then there is an amount of speed as the user starts to shake, but eventually the speed reaches roughly 0 at the end (or half-way) of his first shake, and the process repeats such that each time (or every other time) the speed reaches 0 is the completion of a shake. More complex analysis is able to be implemented to ensure that each shake is properly computed and acquired such as using time, speed, acceleration and directional information acquired by the components. In some embodiments, historical shake information is used to help determine when a shake has occurred. For example, if a user does a shorter motion for his shake, this historical information is helpful in determining that the user's current short motions are each shakes, whereas, when a user with a longer shake motion performs a short motion, it may mean that the shake has not been completed yet. Other information is able to be used to determine when a shake has been performed such as using machine learning and/or template comparison. For example, training occurs by asking and receiving many people's shake movements which enables machine learning to determine multiple different styles of shaking to be used to determine when a specific user makes a motion and whether that motion is a shake. The machine learning is able to be used to learn about a shaking motion in general, and also a specific user's specific shaking motion. The information/feedback from the components is stored by the device.

In the step 3206, the user information/feedback (e.g., motion/movement information) for the shake challenge is analyzed. For example, the user information/feedback from the current shake challenge is compared with previously stored information/feedback from previous shake challenges/training (for the user) to determine if there is a match. Furthering the example, during a training period and/or previous shake challenges, it is determined that the user typically shakes the device by holding the edges of the device while applying a range of 68-72 pounds of grip strength, and the angle of the shake is in the range of +/−5 degrees from vertical, based on the information acquired from the various components. For the current shake challenge, the user's grip strength is determined to be 71, and the angle of the shake is +3 degrees from vertical, so a match is determined. In some embodiments, determining a match is able to include determining if the current information is sufficiently close to the previously stored information. For example, the current information is able to be within a range or within a specified amount of the previously stored information. In some embodiments, multiple classes of shake results are stored since a user may not shake a device the same way every time, and if the current shake is similar to one of the previous shakes, then it is determined to be a match.

In the step 3208, it is determined if the user followed the directions of the shake challenge and if the user's current shaking motion matches previous shake motion information. For example, if the shake challenge requested 5 shakes, but the information provided to the user device is only 3 shakes, then the challenge fails. In another example, based on previous analysis, the user typically shakes with a motion at a 45 degree angle, but the currently detected shakes are roughly vertical, then the challenge fails. However, if the user performed the correct number of shakes and in a manner similar to previously stored shakes, then the user passes the challenge.

When a challenge fails, another challenge is able to be provided, the user's trust score is decreased, the user is locked out of the device, an alert is sent to another device of the user, and/or another action is taken, in the step 3210.

If the user passes the shake challenge, then the user's trust score (or other score) is increased, in the step 3212. In some embodiments, the trust score (or other score) is increased by a certain amount (e.g., 10 points), by a certain percent (e.g., 50%), and/or the trust score is increased to a specified amount (e.g., 90 points or above a threshold). If the trust score is above a threshold after the shake challenge, the user is able to perform a task permitted based on that specified threshold, in the step 3214. For example, if the user was attempting to log in to his social media account, but his trust score was below the threshold for accessing social media accounts, then after the user passes the shake challenge, his trust score is above the threshold, and he is able to log in to his social media account. In some embodiments, fewer or additional steps are implemented. In some embodiments, the order of the steps is modified.

The shake challenge is able to be implemented on a user device and/or a server device. For example, a mobile phone is able to include an application with a shake challenge module. In another example, a server device receives information (e.g., shake movement information) from a user's mobile phone, and the shake challenge application on the server device is able to be used to perform learning and analysis based on the received information of the user's actions. In some embodiments, a server device communicates a request to a user device for the user to perform the shake challenge, and the information received is able to be analyzed at either device.

In some embodiments, device behavior analytics are implemented. For example, device behaviors include: CPU usage/performance, network activity, storage, operating system processes, sensors (e.g., heat), and/or any other device component behaviors. The behaviors are monitored and reported to a machine learning model/system. The machine learning model/system is able to be on the device itself (e.g., user device such as mobile phone) or another device. A filter is able to be used to ensure the machine learning receives appropriate data. Once the machine learning model has been generated/trained, the device is able to monitor the device components in real-time to compare with the model (where the model is the baseline) to detect any anomalies. When the device is behaving in a non-standard way as compared with the model, then the device or the behaviors are considered to be suspicious. If there is suspicious behavior, the device confidence is reduced which lowers the overall trust score of the device/user.

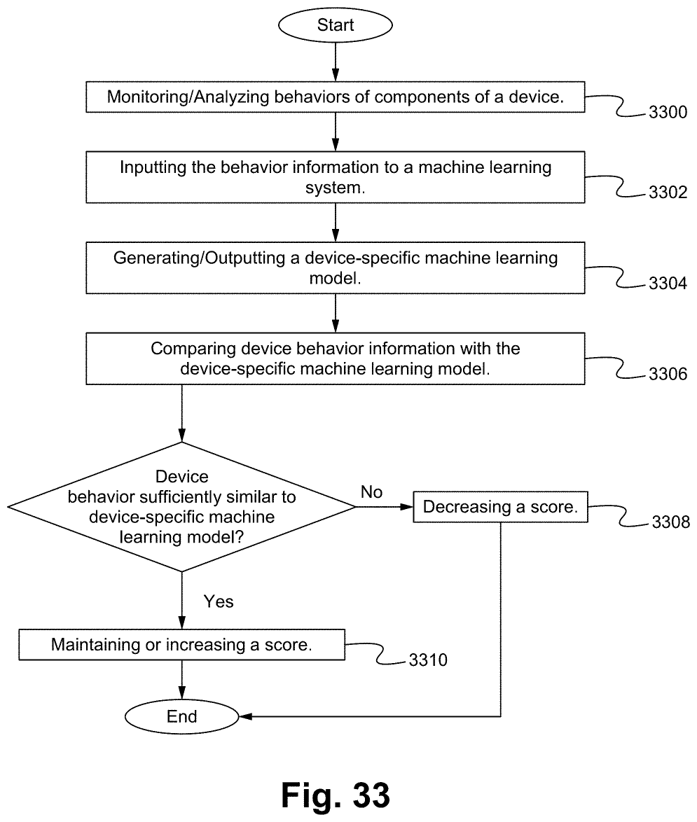

FIG. 33 illustrates a flowchart of a method of implementing device behavior analytics according to some embodiments. In the step 3300, behaviors of components of a device are monitored/analyzed by the device. Device behaviors include: CPU usage, CPU performance, network activity (uploads/downloads), storage (space remaining, change in space remaining, rate of change), operating system processes/applications, sensors (e.g., heat), and/or any other device component behaviors. For example, CPU usage includes analyzing how often the CPU is used, for how long, and what percentage of the CPU's bandwidth is used. CPU performance determines how effectively the CPU is used and if there is a process that is causing a bottleneck in one or more of the components of the CPU that is causing the CPU to slow down. Network activity is able to include uploads and downloads, the speed at which data is uploaded or downloaded, and the amount of data being uploaded or downloaded. Additionally, the sites that the device is communicating with are able to be analyzed (e.g., blacklist/whitelist). Storage analysis is able to be performed such as how much storage space is available, and is a current activity causing the available storage space to decrease (or in particular, decrease at a certain rate). Operating system processes/applications are able to be monitored and analyzed such as the amount of processing bandwidth being consumed and any changes to the system being made by the processes/applications. For example, the CPU bandwidth that a process consumes is analyzed. In another example, an application deleting stored files is monitored. Data from sensors of the device is able to be recorded and analyzed. For example, a heat/temperature sensor monitors the CPU temperature to prevent overheating. In addition to individual components being monitored and analyzed, the interaction of the components is able to be analyzed. For example, the CPU, storage and OS processes are all analyzed together, in addition to being analyzed separately.

In the step 3302, the behavior information/analysis is input to a machine learning system. In some embodiments, the behavior information/analysis is filtered, and the filtered results are input to the machine learning system. For example, if a user accidentally drops his phone, there may be a temporary spike in a pressure sensor or another detected effect; however, this is neither a typical activity of the phone use, nor is it a suspicious activity of the phone, so the data from the phone drop is ignored (e.g., not input into the machine learning system or classified as an event to ignore in the machine learning system). In some embodiments, a behavioral pattern is determined and input to the machine learning system. The machine learning system is able to be stored locally on the device or remotely (e.g., in the cloud). The machine learning system uses any artificial intelligence/machine learning to learn/train the machine learning model. The machine learning system is able to be trained initially and also continuously learn as the device functions. For example, a device's functionality may change after a new application is installed on the device. Moreover, depending on the circumstances, certain levels may be allowable while in other circumstances, those levels may be considered suspicious. For example, when a user is playing a video game on his device which is very CPU and GPU intensive, then 90+% CPU and GPU usage is allowable, and the machine learning model is able to learn that a specific application and a high CPU/GPU usage is allowable. However, when a user is not interacting with his device, and the CPU usage is at 100%, the model learns that such as situation is suspicious.

In the step 3304, a device-specific machine learning model is generated/output by the machine learning system. The device-specific machine learning model is able to be stored locally or remotely, and is able to be continuously updated as learning continues while a user utilizes the device.

In the step 3306, the device behavior information is compared with the device-specific machine learning model. The device-specific machine learning model is able to be used as a baseline to compare for analyzing the device's current behaviors/functionality. The device behavior information is able to be compared with the device-specific machine learning model in any manner. For example, a specific aspect of the device (e.g., a temperature sensor) is compared with the model's temperature data, and if the current temperature is within a range, then the current device behavior is sufficiently similar. Furthering the example, the model's temperature is 85° F. under similar circumstances (e.g., based on the same or similar applications running), and the current temperature is 87° F. which is within an allowable +/−3 degrees of the model's temperature. In another example, the model stores a range of previous temperature readings of 83-86° F., so a reading of 87° F. exceeds the stored range, and may trigger an alert and/or a decrease in a trust score. Similarly, the model stores CPU (statistical) information, network information, storage information, and other information, and the current information is able to be compared with the model to determine if the current information is within an allowable range. As described herein, multiple aspects of the current device are able to be compared with the model simultaneously. For example, the current temperature, CPU usage and bandwidth usage are all compared with the model, and although the temperature is slightly outside of an allowable range, but the CPU usage and the bandwidth usage are well below their respective thresholds, so the comparison is considered to be sufficiently similar. Depending on the implementation, various thresholds/settings are able to be configured to ensure the device behavior analytics are secure, but also properly flexible so that the device does not become unusable.

If the device behavior information is not sufficiently similar to the device-specific machine learning model (e.g., above/below a threshold or outside a range), then a score (e.g., the trust score) for the device is decreased, in the step 3308. The trust score is able to be decreased below a specific threshold or by a certain amount or percentage. In some embodiments, further challenges or tests are able to be provided/taken to increase the trust score. In some embodiments, a determination of suspicious activity triggers additional actions such as shutting down the device.

If the device behavior is sufficiently similar to the device-specific machine learning model, then the score (e.g., trust score) is unaffected or the score is increased, in the step 3310.

In some embodiments, fewer or additional steps are implemented. In some embodiments, the order of the steps is modified.

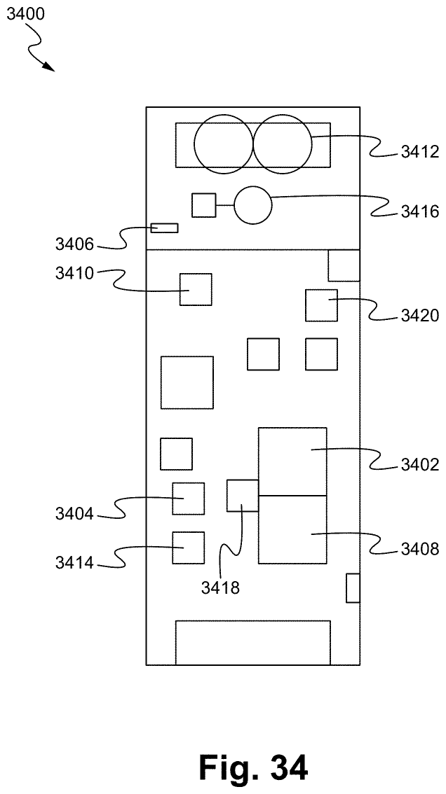

FIG. 34 illustrates a diagram of a device implementing behavior analytics according to some embodiments. Any device components or applications of a device 3400 are able to be monitored such as a CPU 3402, a GPU 3404, networking components 3406, storage 3408 (memory, RAM, disk drives, thumb drives), processes/applications (stored in the storage 3408 or accessed by the device 3400), sensors 3410, microphones/speakers 3412, audio/video/game processors/cards/controllers 3414, cameras 3416, a power source 3418, and others 3420 (e.g., GPS devices, USB controllers, optical drives/devices, input/output device). The device components' behaviors are able to be monitored including: CPU usage/performance, GPU usage/performance, network activity (uploads/downloads), WiFi usage, storage (space remaining, change in space remaining, rate of change), operating system processes/applications, sensors (e.g., heat), audio/microphone usage, audio/video/game controller usage, camera/webcam usage, power usage, GPS/location information, USB controller usage, optical drives/device usage, input/output device usage, printer usage and/or any other device component behaviors.

Many aspects of a CPU 3402 are able to be monitored such as the CPU usage and the CPU performance. The CPU usage varies depending on what processes and applications are running (e.g., in the background and/or foreground). Some applications are high CPU usage applications (e.g., gaming applications and video processing applications). Therefore, a high CPU usage by itself is not a concern. The machine learning system will learn that certain applications are high CPU usage. However, if there is a spike in CPU usage by an unknown application or for an unknown reason, this could be a potential problem. CPU performance is generally related to CPU usage, and if a CPU 3402 is overloaded for some reason, the CPU performance may drop.

A GPU 3404 is a graphics processor which is generally used for applications with high quality graphics such as gaming and other mathematical tasks. Similar to the CPU 3402, the GPU 3404 usage and performance are able to be monitored.

Networking components 3406 are able to be monitored such as available bandwidth, upload/download traffic, WiFi or cellular data, open/in-use ports, and/or any other networking information. Networking information 3406 is constantly changing depending on the applications being used, a user's current browser use (e.g., web pages visited) and many other factors. With machine learning, the system is able to learn how the applications, web pages and other device components affect network usage. For example, a video sharing web page likely uses a significant amount of network bandwidth. In contrast, if a user is playing a non-online video game, then a spike in upload data is a suspicious event that could trigger a decrease in a device's trust score.

Storage 3408 is able to be monitored. Hard drives, memory and/or any other storage devices are able to monitored to determine if any unusual storage is occurring. For example, the amount of space remaining on hard drives and memories is able to be analyzed, as well as the rate that the remaining space is increasing or decreasing. For example, if a new application is installed, then the amount of free space decreases, but once the application is fully installed, the decrease of space stops. However, if a malicious program is trying to corrupt a hard drive, then the free space may continue to decline until the hard drive or memory is full which would cause the device to function less efficiently and possibly stop working. Therefore, if the trust score of the device decreases when it is determined that there is an issue with the storage 3408, then access to the device may be affected which could halt the malicious activity. In some embodiments, access to the device is specific to a program/application/thread such that only a specific application does not have access to the device components, but other applications are still able to access the device components.

Applications stored in the storage 3408 are monitored. The applications are able to be user applications, operating system applications/programs/functions, and/or any other applications. The applications are able to be stored locally or remotely but affect the device 3410. For example, an application stored on a cloud device is able to affect the device 3410. With machine learning, the device 3410 is able to learn how the applications (individually and jointly) affect the different components of the device 3410. For example, the device 3410 learns via machine learning that a video game application utilizes a significant portion of the CPU, video card processing, and network bandwidth and causes the temperature of the device to rise 3° F. When a new application is accessed, installed or executed, the device's suspicion level is slightly elevated (and the device trust score drops accordingly), since there may be changes in other device component analysis when compared with the machine learning model. For example, if a new video processing application is installed and executed, the available storage, CPU usage and temperature are affected (less storage available, higher CPU usage and temperature) when compared with the machine learning model. In response to the change, certain actions may be restricted (e.g., access to online accounts), device functions may be throttled/blocked, and/or a challenge may be provided to the user to confirm the changes/new application. For example, the device 3400 may prompt a user to indicate if there was a known change to the device (e.g., Did you install a new app? or Did you install App A?). If the user confirms that the user installed the new application, then the device trust score is able to be restored to the level before the installation, since it has been confirmed that the change was based on intentional actions of the user. In some embodiments, the device trust score is increased, but slightly below the previous trust score to help protect against a user being tricked into installing malware or other malicious software.

Sensors 3410 monitor a device's status/environment such temperature. If a device's CPU becomes too hot, the CPU could overheat and crash. Therefore, most device's already have an automatic shutdown feature to protect against an overheated CPU. Monitoring the temperature with machine learning is also able to be used to track for suspicious activity such as the CPU's temperature increasing significantly based on visiting a certain web site or using a specific application. The rate with which the device or component temperature changes and/or the overall temperature are able to be monitored. For example, if the temperature of the CPU is rapidly increasing, then the device trust score is able to change and/or the user is able to be alerted. The device is able to take actions to halt suspicious activity without user intervention such as closing an application. With machine learning, the device is able to learn how certain applications/sites affect the temperature and/or other information related to the device, such that the device will be able to detect when an application is acting suspiciously. Applications such as graphic-intensive video games or virtual reality are likely to cause a device's temperature to increase, so the device is able to learn that such types of activities and temperature changes are acceptable. However, a fast increase in temperature when a user visits a web page of a foreign country could indicate that malicious activity is occurring which would decrease the device trust score. The analysis and comparison of the currently detected information (e.g., temperature) with the machine learning model is able to incorporate additional current information. For example, if the current temperature of the device is higher than the expected range of the machine learning model, but it is also determined that the current temperature for the user's location is 100° F., and the user with the device is outside, then this added information is used to account for the elevated temperature (e.g., extend the normal temperature range to 3° F. higher), and not affect the device trust score.

Microphones/speakers 3412 are able to be monitored including which applications are accessing/transmitting the microphone information. For example, if a user has given access to two applications to acquire/transfer microphone-received information (e.g., to make phone calls, to perform voice-based searches), but based on machine learning and monitoring, it is determined that a third application is sending voice data (e.g., microphone-received information), then the device trust score is able to be reduced and/or further actions are able to be taken (e.g., blocking the application, disabling the microphone, blocking outgoing network data).

Audio/video/game processors/cards/controllers 3414 are able to be monitored including processing load, usage, and/or performance. Game processors are generally very powerful processors that hackers are able to utilize to perform malicious tasks; therefore, monitoring gaming processor usage is a valuable tool to ensure the device 3400 is being used properly. Activity of a camera 3416 is able to be monitored including analyzing when content (e.g., images/video) is captured, what content is captured, is the content being shared and/or other activity of the camera. A camera 3416 on a mobile device is able to provide a window into a user's life, and if accessed inappropriately, personal information about a user is able to be stolen and/or shared without the user's knowledge. By ensuring the camera 3416 is only used by the user as desired, a user's privacy is able to be protected. A camera 3416 is also able to be used for other malicious purposes such as overloading the device 3400 (more specifically, the storage 3408) by continuously acquiring content. Via machine learning, the device 3400 is able to determine typical uses of the camera 3416. For example, it is determined the user takes many “selfies” and an occasional video, so when the camera starts being used to acquire and stream continuous hours of video, the device 3400 is able to recognize that there may be suspicious activity occurring. This is also an example of multiple aspects of a device 3400 being monitored and utilized to detect suspicious activity. Specifically, the camera 3416 and network activity are able to be monitored and based on the totality of their activity, the device's trust score may be affected.

A power source 3418 is able to be monitored. The power source 3418 such as a battery is able to be overloaded which could cause the battery to catch fire and/or explode. Battery aspects such as power input, how quickly the battery is draining, capacity, current power storage, and/or any other aspects are able to be monitored.

Other aspects 3420 of the device 3400 are also able to be monitored such as GPS/location, USB controllers, optical drives/devices, and input/output devices. For example, a GPS device which determines a user's location is able to be accessed maliciously to steal a user's location data. Furthering an example, if it is determined that a user sparingly turns on the device's location tracking based on machine learning, but then the device's location tracking is on often or repeatedly, then the device's trust score is able to be decreased and/or the GPS device is able to be disabled.

In an example of a malware attack, a user browses the web or downloads an application which happens to be malware that is configured to provide unintended audio, video and location sharing for a set period of time, and then erase its tracks by deleting the data on the storage and ultimately cause the mobile phone to self-destruct by overloading the battery. Before the malware was downloaded, the mobile phone had a device trust score of 95 (out of 100). The mobile phone via machine learning detects that the microphone, camera and GPS are being accessed by an unauthorized application. For example, the mobile phone knows that only Apps A, B and C have access to the microphone and camera, and Apps C, D and E have access to the GPS, and this malware was never given permission to use any of those devices/components. The mobile phone is then able to take an action after determining that an unauthorized access is occurring such as lowering the trust score of the device and/or halting access to those devices, shutting down those devices, and/or providing an alert to the user on the mobile phone or another device. Since multiple devices are being accessed inappropriately, the trust score is lowered significantly (e.g., below one or more thresholds) which causes the device to limit functionality/access on the device (e.g., shut down devices, prevent sharing of data online). If the machine learning model does not detect the unauthorized access some how, the machine learning model is also able to detect a large amount of data sharing (e.g., network bandwidth usage) which is also able to trigger an alert and lower the device trust score which causes functionality to be limited. The machine learning model is also able to detect that data is being deleted at a higher rate than typical, or specific or protected data is being deleted which is a trigger that the trust score of the device should be lowered and other actions should be taken. Lastly, if the malware was not halted yet, the machine learning model is able to detect a surge of power going to the battery, and turn off the device or take another action before the device catches fire/explodes. Each of the effects of the malware is able to be detected by the machine learning model to prevent further damage/harm.

In some embodiments, suspicious activity is able to be classified as some activities are more suspicious than others. For example, a new application being installed on a device could be a concern, but most of the time a new application is one that the user intended to install, so that would be classified in the lowest suspicion category. An application sharing large amounts of data over a network could be suspicious or relatively benign depending on the typical use of the user. Some video-based influencers share large amounts of video data regularly; whereas, other users may never share video data, so the machine learning model is able to learn based on the specific user's activities. Other activities are able to be classified as highly suspicious such as unauthorized location sharing, surges to the power source, and many more. The classification of the activity is able to affect the device trust score and actions taken.

In some embodiments, there are many actions that are able to be taken when suspicious activity is detected. For example, the device trust score is able to be affected based on the detected activity. When a mildly suspicious behavior/event is detected, the device trust score is able to be decreased slightly (e.g., by 1% or 1-3 points), whereas a medium-level suspicious behavior decreases the trust score by 5%, 5-10 points or below a top threshold, and a high-level suspicious behavior decreases the trust score by 50%, 50 points or below the lowest threshold. Therefore, if the user installs one new application, the device score may go from 95 to 94, which would not have any practical effect in terms of device functionality. However, if the user attempts to install 20 new applications, the device score may drop from 95 to 80 (with 1 point drops for each of the first 15 applications), and if the threshold for download/installation functionality is 80, the device may be paused from installing the last 5 applications. In addition to or instead of affecting device functionality, the device is able to perform additional actions automatically or with user input/assistance. For example, the device is able to prompt a user to confirm the desired changes (e.g., You have installed 15 applications recently, are you trying to install more? Y/N). The device is able to automatically shut down components or the entire device. For example, if an attack on the device's storage or power source is occurring, the entire device is able to shut down. In another example, if data is being shared over the network, then WiFi, cellular or other networking access is able to be turned off In some embodiments, multiple thresholds are implemented such that if the device trust score is above a highest threshold (e.g., 85), then there are no limitations on access/functionality, but if the device trust score is between 75 and 85, then certain access/functionality is limited (e.g., files are not allowed to be deleted, or data is not able to be uploaded/shared), and if the device trust score is 75 or lower, then access/functionality is severely or completely limited (e.g., the device is only able to perform basic functions). Any number of thresholds and limits to access/functionality are able to be implemented.

The device trust score described herein is able to be used in conjunction with the other trust scores to generate an overall user/device trust score.

Homomorphic encryption enables a user/device to perform computations on encrypted data without decrypting the data. The biometric data (or other data) described herein such as fingerprints, face scan, microtremors, gait, shake motion, and many more, is able to be encrypted and stored using homomorphic encryption such that the homomorphically encrypted data is able to be queried for specific biometric data without decrypting the data. In some embodiments, the homomorphically encrypted data becomes a user's password or token (or is used to generate the token). An exemplary query is: “does this match with the gait pattern?”. A system with the homomorphically encrypted data is able to return a response to the query such as “yes” or “no.”

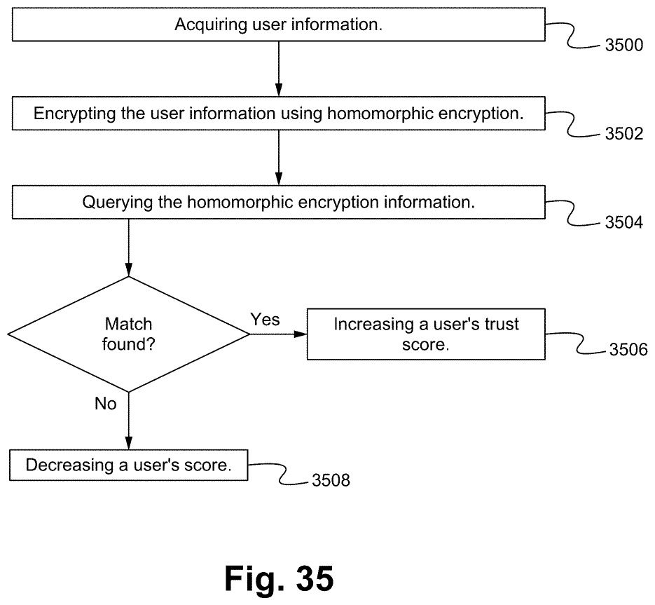

FIG. 35 illustrates a flowchart of a method of utilizing homomorphic encryption according to some embodiments. In the step 3500, user information is acquired. As described herein the user information is able to include behavior/biometric information such as microtremors, gait, a shake motion, joint vibrations, temperature, and other data specific to a user. The behavior/biometric information is able to be acquired in any manner such as the user holding a device, and the device detecting and recording data (e.g., using a pressure sensor to detect a user's grip of the device, or using accelerometers and gyroscopes to determine a user's gait). In some embodiments, instead of or in addition to acquiring behavior/biometric information, other user information is acquired. In some embodiments, the user information is stored in a database or other data structure. For example, each behavior (e.g., gait) is stored in its own class/classification. The user information is able to be continuously updated/modified depending on the user actions. For example, as the user continues to use his device, user information is acquired. Machine learning is able to be used to update the information and continuously learn about the user.

In the step 3502, the user information is encrypted using homomorphic encryption and stored. In some embodiments, the encryption is Partially Homomorphic Encryption (PHE), Somewhat Homomorphic Encryption (SHE) or Fully Homomorphic Encryption (FHE).

PHE enables sensitive data to remain confidential by only allowing select mathematical functions to be performed on encrypted values. Only one operation is able to be performed an unlimited number of times on the ciphertext (e.g., addition or multiplication). Examples of PHE include ElGamal encryption (uses multiplication) and Paillier encryption (uses addition).

SHE enables limited operations (e.g., addition or multiplication) up to a certain complexity, where the limited operations up to a specified complexity are only able to be performed a set number of times.

FHE enables using any computable functions (e.g., addition and multiplication) any number of times which enables secure multi-party computation.

In the step 3504, the homomorphic encrypted information is queried for comparison purposes. The query is able to be implemented in any manner such that the encrypted information remains encrypted during the query. For comparison, an unencrypted database is searchable/queried for a specific content item (e.g., text, image). Similarly, a homomorphic encrypted database is able to be queried (however, without decrypting the database). The encrypted querying is able to be implemented in any manner. For example, the query includes an XOR operation to determine if a match is able to be found between current (newly acquired) user information and the stored, encrypted information. More specifically, the XOR operation is used to compare current user information (or a subsection of the current user information) with the stored encrypted information. In another example, a content item to be searched for (e.g., current information) is encrypted using the same homomorphic encryption as the stored information, and then the current information and the stored information are compared using the XOR or other operation to determine if the same content item is in the homomorphic encrypted data store (e.g., database). By XORing the current information with different segments of the stored information, a match is found when the result of the XOR is 0. In another example, the homomorphic encrypted data store includes gait information which is able to include specific vector information such as speed and direction of a user and the user's arms, from when the user previously walked with the device. Then, when current gait information is acquired as the user is walking, the acquired gait information is compared with the stored homomorphic encrypted gait information. In another example, facial recognition information is encrypted and stored using homomorphic encryption. Then, the user is prompted for facial recognition information again for access, and the acquired facial information is encrypted using homomorphic encryption and then compared with the stored facial recognition information. The query/comparison is able to compare the current user information with stored, homomorphic encrypted information without decrypting the stored homomorphic encrypted information.

In the step 3506, when a match is found, a user's trust score is increased or remains the same. For example, the current behavior/biometric information is compared with the stored homomorphic encrypted behavior information, and when a match is found, then the user's trust score is increased (e.g., above a threshold) or maintained (e.g., if the user's trust score is already above a threshold or at a maximum). In some embodiments, in addition to or instead of affecting a user's trust score, access is granted to a service. For example, a user attempts to log in to his social network account, and if a match is found, then the device and/or system grants access to his social network account. The access is able to be granted in any manner such as generating/providing a token to the social networking system which permits access to the user's account.

In the step 3508, if a match is not found, then a user's trust score is decreased. For example, the current behavior/biometric information is compared with the stored homomorphic encrypted behavior information, and when a match is not found, then the user's trust score is decreased (e.g., below a threshold) and/or more behavior/biometric information is acquired/analyzed. In some embodiments, in addition to or instead of affecting a user's trust score, access is denied to a service when a match is not found.

In some embodiments, fewer or additional steps are implemented. As described herein, when a user's trust score is above a threshold, access is provided to the user on the device. In some embodiments, the order of the steps is modified.

The comparison of the current user information and the stored homomorphic encrypted information is able to be performed on the user device (e.g., mobile phone), a server/cloud device, another device and/or any combination thereof. For example, on the user device, the user device stores the encrypted information and then compares the current user information. In another example, the server device receives user information from a user device, encrypts the user information (or the user device encrypts the user information) using homomorphic encryption, stores the encrypted information, and then compares newly received user information (which is encrypted either at the server or the user device) with the stored encrypted information. The server is then able to take an action such as providing an access token, providing access to a service in another way, and/or adjusting a user's trust score based on the query/comparison of the stored encrypted information and the new/current user information.

Voice analytics are able to be used for user identity verification. A human voice changes in different environments, performing various activities or with various user moods. The voice has different tones such as warm, clear, soft, scratchy, mellow, or breathiness. These tones may relate to different user moods such as anger, calmness, stress, or excitement. Voice qualities also include: pitch, vocal fry, strength, rhythm, resonance, tempo, texture, inflections, and others. For example, a person's voice changes, often to a great extent, in different situations such as talking on a phone, giving a speech, conversing with a close friend, talking in a business meeting, walking, running, exercising, and others. These differences in voice quality and/or voice changes vary widely for individuals and add a great degree of identifiability for specific users.

User identification traditionally was done using Voice Print Analysis. This is currently a common technique, and as such it has been researched and currently is vulnerable to spoofing using various methods.

The method described herein is able to immediately identify a user using real-time machine learning of voice patterns in various situations on an ongoing basis. Requiring a user to purposely speak to a device to identify themselves is not required and is both an undesirable user experience and is a security exposure to automated software attacks or manual malicious activities.

Additionally, voice quality factors are able to be related to other monitorable human factors such as heart rate, physical movements and motion analytics such as gait and others. Moreover, a person walking or running has different vocal qualities than someone at rest. This both allows multiple factors to be related to increase security as well as guaranteeing that the user is human and not malicious software, pre-recorded voices, and so on.

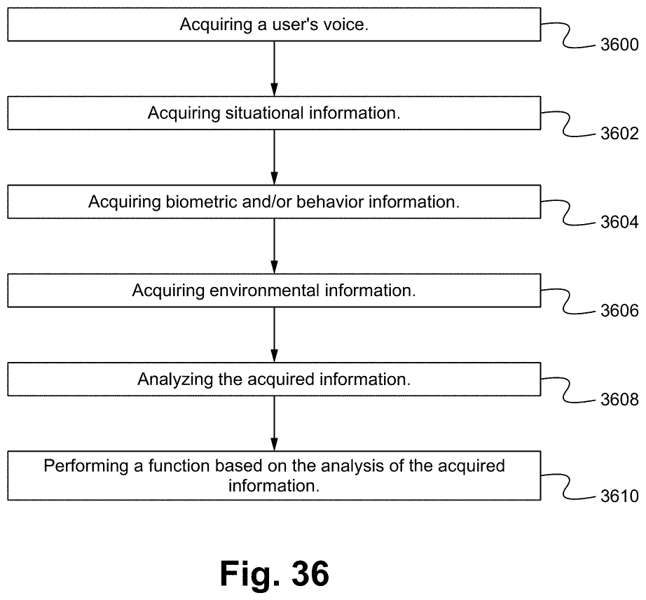

FIG. 36 illustrates a flowchart of a method of implementing user identification using voice analytics according to some embodiments. In the step 3600, a user's voice is acquired. The user's voice is able to be acquired in any manner such as via a microphone in or coupled to a device. The device is able to be any device such as a mobile phone, a wall-attached device, an IoT device, and/or any other computing device. In addition to acquiring a user's voice, situational, biometric/behavior, environmental and/or other information is able to be acquired. The additional information is able to be acquired in conjunction (e.g., at the same time) with the user's voice information.

In the step 3602, situational information is acquired. The situational information is able to be acquired in any manner such as by: using the microphone/camera of the device, accessing the user's schedule/calendar, accessing Internet data, accessing application data, and/or another manner. For example, when a user makes a phone call using the phone app on the mobile phone, the application information is able to be acquired. In another example, a user's calendar information is able to be analyzed based on the current time to determine that the user is currently speaking at a meeting or providing a speech.

In the step 3604, biometric and/or behavior information is acquired. As described herein, a user's biometric and behavior information is able to be acquired when the user utilizes the device. For example, when the user walks, the user's arm movements, microtremors, and gait information are able to be acquired, and when the user performs another activity, the specific motions and details are able to be acquired using the sensors and/or components of the device. The biometric/behavior data is able to be acquired using a wearable device such as a smart watch which is able to acquire a user's heart rate and/or other physical information.

Biometric information such as a face scan, 3D face scan, ear scan, fingerprints and/or other information is able to be acquired while a user is talking. For example, if the user's voice is detected via a microphone, a camera of a device is able to be directed at the user's face, ear, or other body part to acquire facial information for a facial scan to further confirm that the user is the authorized user.

In the step 3606, environmental information is acquired. The environmental information is able to be acquired in any manner such as by: using the microphone/camera of the device, using sensors of the device (e.g., a temperature sensor), accessing Internet data (e.g., weather web site), accessing application data, and/or another manner.

In some embodiments, some or all of the steps 3600, 3602, 3604 and 3606 occur simultaneously or nearly simultaneously. The acquired information is able to be stored in any manner to be processed/analyzed. In some embodiments, a device or devices acquire the information described herein without the user actively utilizing the device. For example, a temperature sensor is able to detect and indicate that the current temperature of the room is 90 degrees versus a different room which is 60 degrees. In another example, a device is able to acquire the temperature information in a location by accessing the information from a weather web site.

In the step 3608, the acquired information (e.g., voice information, situational information, biometric/behavior information, environmental information) is analyzed. The acquired information is able to be analyzed in any manner such as using machine learning to detect patterns for learning and for comparisons (of acquired information with stored information) to determine if the current user is the authorized user.

Analyzing the voice information includes analyzing the tone of the voice, the mood of the user and/or other voice qualities. Analyzing a user's tone of voice is able to be performed in any manner such as using machine learning to compare the voice with other voice's that have been classified by tone such as warm, clear, soft, scratchy, mellow, or breathiness. A user's voice is able to be mathematically compared using tonal patterns and/or other data. The tones may relate to different user moods such as anger, calmness, stress, or excitement. Therefore, using a relational database, machine learning and/or another organizational structure/system, the user's voice/tone is able to be correlated to a user's mood. Voice qualities are also able to be analyzed such as pitch, vocal fry, strength, rhythm, resonance, tempo, texture, inflections, and others. The voice qualities are able to be analyzed in any manner such as comparing a user's voice or aspects of the user's voice with stored voices that have been classified. For example, pitches are able to be classified as high, low, and in between or in different groupings. Pitch is able to be determined based on frequency such as high frequency above a certain amount (e.g., 880 hertz) and low frequency below a certain amount (e.g., 55 hertz). The other voice qualities are able to be analyzed and compared using other audio analysis. The voice analysis is able to determine if the user's voice is the authorized user by comparing acquired information and stored information to determine if there is a match (e.g., a pattern and/or any other audio comparison/matching).

The analysis is able to be used to determine a user's situation. For example, a person's voice changes, often to a great extent, in different situations such as talking on a phone, giving a speech, conversing with a close friend, talking in a business meeting, walking, running, exercising, and others. These differences vary widely for individuals and add a great degree of identifiability for specific users. Additionally, languages, dialects, accents, lisps, and/or any other distinctions of a voice are able to be analyzed and learned, as they are useful distinguishing factors. Specific pronunciation distinctions are able to be detected and learned. For example, if a user emphasizes a different syllable of certain words than other people, this could be a helpful distinguishing factor when analyzing the user's voice.

In some embodiments, the content and/or style of the voice information is analyzed. By continuously acquiring and learning from a user's voice, the device is able to determine/learn specific words, phrases or speaking styles that the user uses. Some examples include: a user may say the word “like” or the phase “you know” often (e.g., at least once every 5 words or after 90% of sentences); a user pauses for roughly two seconds after each sentence; a user speaks rapidly without ever pausing for more than half of a second; a user speaks with a detected cadence or rhythm; and/or a user may commonly refer to movie quotes. In another example, vocabulary levels/classes are able to be generated based on words/phrases, and a user's speech is able to be classified based on the words/phrases the user utilizes. For example, a person with an advanced degree will likely have a different vocabulary than someone with much less education, so the vocabulary used is another distinguishing factor when performing voice/language analysis. A user's vocabulary is able to classified in classifications such as levels 1-10 (e.g., level 1 is kindergarten level and level 10 is advanced degree level vocabulary) or some other classification.

Analyzing the situational information includes determining relevance of information to a current situation. For example, based on a current time/date, a user's calendar is able to be analyzed to determine if any meetings or other events are scheduled. A user's voice may be different at a business meeting when compared with a personal lunch with a friend. Similarly, for some people, giving a speech is a stressful event which would cause a user's voice to be different. The user may have an exercise schedule (in the calendar), or it is able to be determined that the user's current location is a gym based on GPS, or it is able to be determined that the user is running by analyzing the user's current speed and location. The camera of a user's device is able to detect exercise equipment and/or movements that indicate exercising. Machine learning is also able to determine that the user walks/runs/exercises at a same/similar time each day. A user's voice is likely to be different while exercising (e.g., more winded). A user's voice is able to be different based on the current situation the user is in, and the different situations and corresponding voice differences are able to be analyzed and learned. Analyzing the situational information also includes determining relationships or correspondences between acquired situational information and the acquired voice information. The relationships/correspondences are able to be learned (e.g., when a user walks, his voice is similar to when the user is at rest (or slightly winded), but when the user runs, his voice sounds more winded, has more pauses and/or any other effects). In some embodiments, the relationships/correspondences are learned by analysis of all of the users of the system, and then refined for the specific user. For example, if it is typical for most users to be winded when they talk and run (based on analysis of all the users), then it is likely that the user will be winded when he talks and runs. Once, the user has been talking and running enough times, the device learns the specific correlation between running and talking for the user.

Analyzing the biometric/behavior information utilizes information acquired using device components such as gyroscopes, sensors, cameras, and/or any other components. As described herein, the acquired information is able to indicate user actions or behaviors such as walking, running, exercising, driving, and many more. The behaviors are able to be recognized and used to determine if the behavior affects the user's voice. A user's behavior is able to affect his voice as described herein such as when the user is walking or running. For example, running causes the user's heart rate to increase or the user to be out of breath, which affect the user's voice. In some embodiments, a user's voice is classified based on the behavior (and/or other categories) such that a user's voice for no activity is classified in a different classification than a user's voice while running. Any number of classifications and sub-classifications are able to be implemented. The behavior information is able to be analyzed with the situational information. For example, situational information such as a calendar appointment may indicate that the user runs at 5 a, and if the sensors indicate that the user is making movements that correspond with running movements at 5:05 a, then there is more certainty that the user is running.

Analyzing the environmental information utilizes information acquired using sensors and/or other sources. For example, a temperature sensor of a device indicates that the ambient temperature is 100° F. A user may be more tired based on the current temperature and/or humidity which could affect the user's voice. Similarly, if a user is very cold (e.g., temperature sensor indicates 0° F.), the user's teeth may chatter a little, which affects the user's voice. Other environmental factors are able to affect a user's voice such as being in a smoky room which could cause a raspy/coughing voice, a dark room where the user whispers instead of speaking normally/loudly, a very loud room (e.g., a concert or party) where the user speaks more loudly than usual, and/or any other environmental factor. By knowing the environmental information, the device is able to account for the differences in the user's voice. For example, if the light sensor or camera of the device determines that the user is in a dark room, the device is able to analyze the user's voice as a whisper instead of comparing the user's voice with a normal voice. Furthering the example, a user's whisper is stored/learned and compared for situations when the user whispers. The different environments and the corresponding voices are able to be classified based on the environment (e.g., a classification for darkness, a classification for hot weather, and so on).

In addition to analyzing the various information separately, the information is also able to be analyzed together. For example, a user running on a cold morning in winter while talking on his phone may have a different voice than the same user running on a hot summer day while talking on his phone. The situational, biometric/behavior and environmental information are all able to be analyzed along with a user's voice to better identify the user based on the current situation, behavior, and/or environment. The analysis of the information includes processing, sorting/classifying and comparing the information. For example, newly acquired voice information (and any accompanying additional information) is compared with stored/learned information to identify the user. Furthering the example, the user attempts to log into a social networking site, and the user's voice is going to be used to gain access. The user has been talking on the device while sitting in the office, and the voice matches the stored voice information (e.g., using a voice matching algorithm and/or any other audio comparison implementation). Since the device recognizes the user as the authorized user, the device is able to access the social networking site.

In some embodiments, voice changes based on the additional information (e.g., situational, behavior, environmental) are analyzed. For example, Person A and Person B may have similar voices in terms of pitch and other voice qualities while at rest, but Person A is physically fit and is able to run and talk with minimal change, whereas person B struggles to talk while running, thus the change of voice from resting to running is able to be detected and analyzed. In another example, Person X is uncomfortable with public speaking (e.g., causes a jittery/trembling voice) while Person Y is an eloquent public speaker, so the change from rest to a business meeting or a public speech is able to be detected and compared, and if Person Y tried to use Person X′s device, the device would be able to detect the difference in the change of voice.

The analysis of the information is also able to include learning from the information. For example, machine learning is continuously implemented on the device such that any time the user speaks, the device acquires, analyzes and learns from the information. Additionally, the device learns any contextual information such as situational, behavioral, environmental, and/or other information. Based on the machine learning, the device is able to identify the user based the user's voice and any related information.

In the step 3610, a function is performed based on the analysis of the acquired information. The function is able to include providing or denying access (e.g., to the device, a web site, a social networking account, a bank account, a door, and/or another object/service). The function is able to include adjusting the user's trust score on the device. If the user's voice matches previously stored information based on the analysis, then the user's trust score is maintained or increases and/or access may be granted to a service. If the user's voice does not match the stored information, then the user's trust score is decreased and/or access may be denied to the service. In some embodiments, how close the match is, affects the adjustment of the trust score (e.g., an exact voice match increases the trust score by 10 points or above a top threshold, but a slightly similar voice only increases the trust score by 2 points or above a second level threshold). In some embodiments, performing a function includes generating a token. For example, the token is able to include authorization to access a device and/or service.

In some embodiments, fewer or additional steps are taken. For example, in some embodiments, the environmental information is not acquired or analyzed. In some embodiments, the order of the steps is modified.

As described herein, human activities are able to be identified and monitored with modern smartphone and personal device hardware. These devices have extremely accurate sensors which can detect minute movements, motions, environmental factors, locations, sound, light and various other human conditions and activities.

Each human activity will correspond to several other human measurable conditions. The relationship of heart rate and breathing rate will relate to the activity, such as breath rate and running. Similarly, the voice quality will correspondingly change due to activities.

By monitoring in a real-time machine learning model, the pattern of human physical responses corresponding with external activities are extremely identifiable and unique to each individual.

The user identity can be established immediately without requiring the user to manually identify themselves to an identity challenge. The system can guarantee that this user is a human and not any form of malware, human hacking attempt or other manual or automated attempt to misrepresent the user's identity.

The ultimate value is to reduce or eliminate identity fraud of any form.

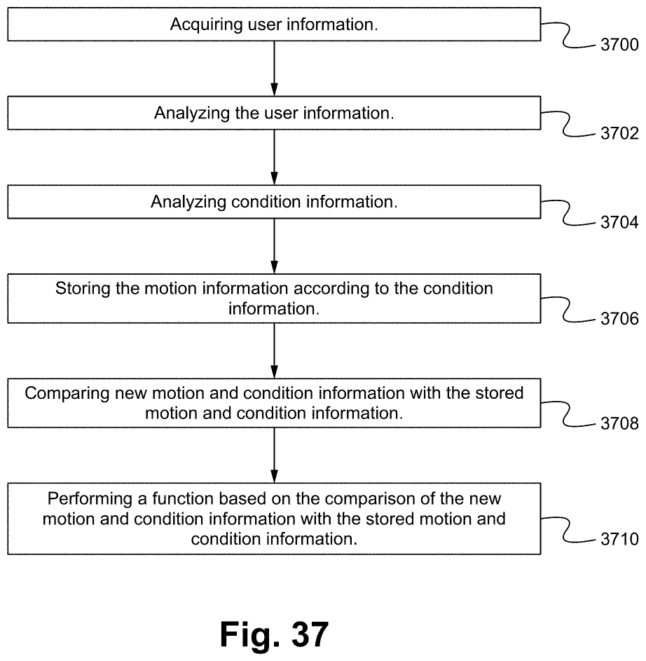

FIG. 37 illustrates a flowchart of a method of using a multitude of human activities for user identity according to some embodiments. In the step 3700, user information is acquired. The user information is acquired using the device in any manner. The user information is able to be acquired using components of the device (e.g., an accelerometer, a gyroscope, a sensor, a microphone, a camera, a GPS). For example, a mobile phone is able to be used to acquire motion information, voice information, image/video information, and/or other information using the components of the phone.

In the step 3702, the user information is analyzed/processed to determine motion information and classify the motion information. The user information is able to include different motion aspects which are able to be collected and analyzed. For example, when a user is lying down, the device is stationary, and the gyroscope may detect a certain orientation depending on where the device is being held. Furthering the example, if the device is in the user's pocket, the device is able to detect that it is parallel with the ground. Additionally, the device is able to use a heat sensor to determine that the temperature is higher than the ambient temperature outside since the device is next to the user's body in a pocket, as opposed to on a table where the temperature and orientation would likely be different. Similarly, if the user is in a car, the device may be stationary according to the accelerometers in the device since the device is not moving in relation to the car, but the GPS is able to detect that the device is moving 60 miles per hour, so it is able to be determined that the device is in a car. Additionally, other analysis is able to be performed to determine which car the device is in (e.g., does the device have access to communicate with the car; if yes, it is the phone owner's car, and if not, then it is another person's car). As described herein, body movements are able to be detected by the device and the components of the device such as determining that the user's legs are moving with the device in his pocket, so it is known that the user is walking. Multiple components are able to be used together to determine the current motion such as the accelerometers, gyroscopes and GPS to determine that the user is walking versus running based on overall speed and/or leg motion speed. Standing, lying down, sleeping, walking, riding a bicycle, driving in a car and many other actions, all have distinguishing motions.

The analyzed motion information is able to be classified. Based on rules, pattern matching or another form of machine learning each motion or a group of motions are able to be classified. For example, movement back and forth, within a range of motion, within a range of speeds is able to be classified as walking (e.g., as the device in a user's side pocket moves back and forth or based on a user's arm swinging while wearing/holding a device). In another example, certain vibrations and other movements from a user walking are able to be detected for when a user's device is in his back pocket. Another classification is able to be when the device is detected at speeds that would indicate the device is in a vehicle (e.g., above human thresholds or other patterns that indicate a vehicle). The different rules/patterns/aspects for each classification are able to input and/or learned based on training behavior and/or any other learning implementation. Although some rules/patterns/aspects have been described herein for certain actions, many other rules/patterns/aspects are able to be detected and learned.

In the step 3704, condition information such as voice/sound information (e.g., background noise), image/video information, situational information, environmental information and/or other information (e.g., location) is analyzed for further classification. The conditions correspond with motion information in order to provide further classification of the detected/analyzed motion information.

In the step 3706, a motion and condition data structure stores the analyzed motion and condition information. For example, the motion and condition structure is a matrix with motion classifications crossed with condition classifications. For example, the standing “motion” is a classification which can be affected by various conditions such as background noise, temperature, voice, and/or other conditions. In another example, a user's gait when he first wakes up is different from his gait at the office which is also different from his gate at the park where there are ducks to avoid. In other words, instead of simply having a general gait analysis, a user's gait is analyzed based on the current circumstances, so a much more refined analysis is performed which is much harder to corrupt/spoof. Stored in each cell of the matrix (or other data structure) is the motion information/pattern and/or other information. For example, a user's gait information in the early morning is stored in the cell that matches up with “gait information” and “in the morning,” while the user's gait information at the park is stored in the cell that matches up with “gait information” and “at the park.” Using a matrix or other data structure of classifications, the device is not only able to match the current user's information with stored user information, but the device is also able to avoid a being tricked/spoofed by malware, since the recognizable pattern changes often. Machine learning is able to be used to continuously update the data structure.

In the step 3708, the motion and condition data structure is used to determine whether the user is the authorized user. As described herein, motion information and condition information are acquired and compared with stored information. The comparison is able to be performed in any manner such as pattern matching and/or any other artificial intelligence analysis.

In the step 3710, a function is performed based on the user authorization analysis. For example, if the user is authorized, then access is granted to a device/service and/or the user's trust score is maintained or increased. If the user is not authorized (e.g., a match is not found), then the access is denied and/or the user's trust score is decreased.

In some embodiments, fewer or additional steps are implemented. In some embodiments, the order of the steps is modified.

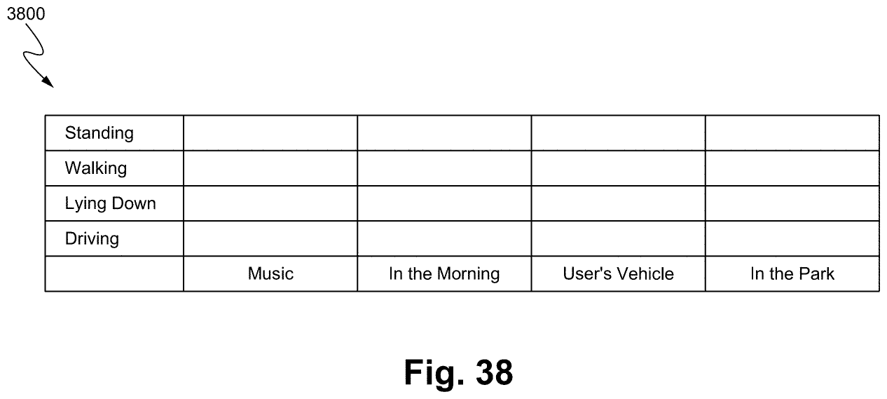

FIG. 38 illustrates a diagram of an exemplary motion and condition data structure according to some embodiments. The motion and condition data structure 3800 includes rows and columns of motions and conditions. For example, the labels of the rows are motions, and the labels of the columns are conditions. As shown in the example, motions include standing, walking, lying down, and driving. As also shown in the example, conditions include background music, in the morning, user's vehicle, and in the park. The motion and condition data structure 3800 is able to change (e.g., expand) based on acquiring new information. For example, if it is determined that the user walks differently after exercising, then the motion and condition data structure 3800 is able to be expanded to include the “after exercising” column. In another example, the motion and condition data structure 3800 is able to be expanded to include a “running” row.

In some embodiments, each motion has its own data structure with a plurality of corresponding conditions. For example, a walking motion data structure includes conditions such as with background music, at wake up, at park, and others. Other data structures are able to be generated and utilized for other motions as well. In some embodiments, there is a data structure for each condition.

In some embodiments, multiple motions are included in a single row or are cross-correlated. For example, one row specifies walking, and a second row specifies walking+exercising, as the user's motions and other information are able to be different under the two different scenarios. In some embodiments, multiple conditions are included in a single column. For example, one column specifies “in the morning,” and a second column specifies “in the morning” +“below 50 degrees,” as the motion information and other information are able to be different under the two different scenarios.

The data structures are able to store patterns or other motion information with the corresponding conditions. For example, each time the user walks, machine learning is used to learn the user's walking motions. Additionally, the location, time of day and/or other information is able to be acquired to further classify the stored motion information. Then, when the user performs a similar motion, his motion is able to be compared with the previously stored motion under the same or similar circumstances/conditions, for a very accurate comparison.

A roaming user password is able to be based on human identity analytic data. Human identity analytic data is collected with various techniques. These include: motion analytics, voice print and quality, live facial scans, breath print and quality analysis, gait analysis and many others. The analytics are able to be used to generate a matrix of data values (e.g., motion and condition information). The matrix is able to include the baseline analytic value, the quality or confidence score of the analytic, analytic class and unique code, priority, and/or other information. The matrix (or other data structure) is able to uniquely identify the user through a multitude of identity analytics. The matrix is able to be stored locally and/or remotely (e.g., in the Cloud). By storing the matrix in a central repository in the Cloud, any authorized device is able to access/communicate with the matrix for identification analytics purposes.

There are cases where some of the analytics are of low value or are invalid. This is identified within the user data. The user is able to be identified by a preponderance of valid analytics (or another threshold).

The following are examples of analytics determined to be invalid/failed: gait analysis failed because of injuries or environmental factors, facial scan fails because of facial coverings, such as surgical masks, and facial scan fails because of low-light conditions. The following are examples of analytics determined to be valid/pass: breath pattern and quality success, voice pattern and quality success, a recent shake challenge success, and other motion analytics success.

With enough or a preponderance of success analytics, the user can be identified with great accuracy.

The user analytics data is able to be used as a unique password but is extremely sensitive data and is never be exposed.

The analytics dataset is able to be processed into a 1-way hashing algorithm which, even if exposed, does not expose the actual human analytics data. This 1-way hashed data is then able to be used as a central password repository. The user analytics, processed into the 1-way hash are then able to be compared with the central version to authenticate the identity of the user.

A use-case for this technology is for stationary access and identity devices. A device (such as a smartphone or tablet) is able to be mounted on the wall. Many of the sensor and monitoring systems in the stationary device are able to uniquely identify the user by: live facial recognition, voice print and quality, gait, breath, and/or many others. The device is able to monitor and collect user characteristics. The characteristics are able to then be processed into a 1-way hash and compared to a central analytics password repository (e.g., in the Cloud). The solution supports external (not personal) devices to uniquely identify users using a multitude of factors. Exemplary uses include: entry systems, for financial transactions with virtual notary systems included automatically, and/or for a multitude of other use cases.

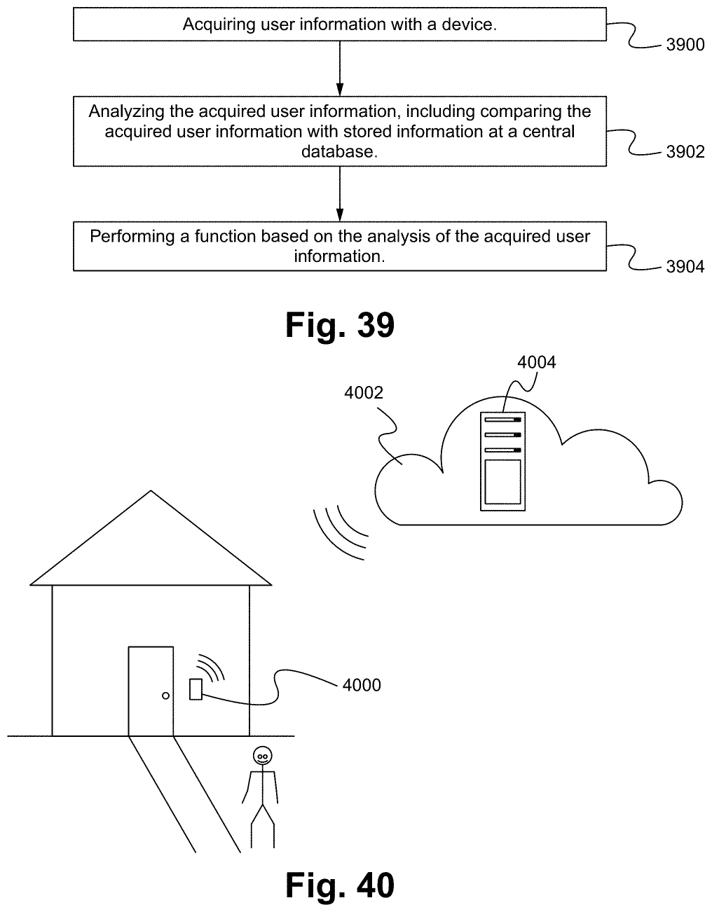

FIG. 39 illustrates a flowchart of a method of implementing a roaming user password based on human identity analytic data according to some embodiments. In the step 3900, a device (e.g., a mobile device positioned on a wall or next to a door, or a security system) acquires information of a user. The device is able to acquire the information in any manner such as acquiring video information using a camera, acquiring audio information using a microphone and/or any other sensors to acquire other information.

In the step 3902, the acquired information is analyzed/processed. Analyzing/processing the information is able to include image processing, video processing, audio processing and/or any other processing to separate and classify the different aspects of the acquired information. For example, the user's voice is classified as voice information, the user's leg and arm movements are able to be classified as gait information, and the user's facial, head, ear and/or any other biometric information is able to be classified as biometric information. Further processing is able to take place to analyze each aspect (e.g., processing the leg and arm motions to determining specific characteristics of the user's gait). Additionally, if there is condition information that accompanies the user information, the condition information is able to be acquired as well or is able to be used to further classify the user information. For example, a user may have a different gait in summer versus winter since the user's clothes may affect his gait.

Analyzing/processing the information is able to include comparing the acquired information with stored information. In some embodiments, the stored information is stored in a central repository accessible by the device (and other devices such as Internet-connected devices). In some embodiments, the stored information and the acquired information are stored such that the underlying data is unreadable (e.g., hashes of the information are stored and compared). For example, previously acquired information is stored as hashes, and currently acquired information is hashed to be compared with the stored hash information. In another example, encrypted information is able to be compared (e.g., the stored and the currently/recently acquired information are encrypted and compared. Any form of securing the data, but also allowing the data to be compared is able to be implemented.

The comparison is able to include many aspects/details of the acquired information. For example, biometric recognition, voice recognition, motion analysis, gait analysis, breath analysis and other analysis are able to be implemented. Each separate aspect of the acquired information is able to be used for comparing with the stored information, and each separate aspect is able to receive an identification score and/or a pass/fail. By performing multiple forms of analysis, the chances of tricking a system are decreased, and the confidence of accurately identifying a user is able to be increased. For example, if a device recognizes a user's face (using 3D live facial recognition), voice and gait, then the likelihood that the user is identified correctly is very high, whereas a facial recognition-only system is able to be tricked by a simple picture. However, if the device recognizes the user's face, but the voice and the gait do not match with the stored information, then the user may not be considered as identified. Any implementation is able to be used to determine whether the user is identified. For example, each aspect of user information is analyzed and receives an identification score, and the identification scores are combined to generate a total identification score, and if the total identification score is above a threshold, then the user is considered identified; otherwise, the user is not identified. In another example, where a match of each aspect is either found or not, a user is identified if more aspects are found than are not found. For example, if ear identification is a fail due to the user wearing a hat, but voice identification is a pass, gait identification is a pass, and breath identification is a pass, then there are 3 passes and 1 fail, which ultimately is a pass (or the user is considered identified). In another implementation, identification of a user occurs if there is a number above a threshold of aspects that match/pass (e.g., a threshold of 5 aspects that match). In some embodiments, the identification process includes searching for matches of a user based on the acquired user information. In some embodiments, the search continues after a user is identified to ensure no other users are identified as well, and if the user is identified as two or more people, then the user is not considered identified. Other processes/tasks are able to be implemented to clarify identification.

In the step 3904, a function is performed based on the analysis of the acquired information. The identification aspect is able to be implemented in conjunction with other security systems/features. For example, for security system such as enabling a user to unlock a door or enter an area, the security system/door lock includes a list of people who have authorization for that area (e.g., in an accessible database), so if a user is identified and is on the list of authorized people, the user will be able to enter that building. Furthering the example, if the user is identified by the device and the user is listed as having access to a building, the device is able to send a signal to unlock/open a door. In another example, if the user is not identified by the device (e.g., the user fails 2 of the 3 identification tests), then the device does not send an unlock/open signal to the door. The function performed is able to be access to a device, service and/or any other system. In addition to gaining access to a door/building, once a user is identified, services within the building are made available to the user.

In some embodiments, fewer or additional steps are implemented. In some embodiments, the order of the steps is modified. For example, the central repository of stored user information receives the user information from devices. The user information is able to be received from wall mounted devices during a training period. The user information is able to be received from user devices (e.g., a user's mobile phone is able to be used to acquire the user information described herein and send the information (or a hash of the information) to be stored in the central repository).

FIG. 40 illustrates a diagram of a system implementing a roaming user password based on human identity analytic data according to some embodiments. A device 4000 is configured to acquire user information. As described, the device 4000 is able to include a camera, a microphone, sensors, and/or other components to acquire the user information while the user approaches the device 4000 (e.g., while the user walks toward the device). The device is able to be a fixed device (e.g., embedded within or attached to a wall) or a removable device (e.g., temporarily affixed to a wall). The device 4000 is configured to communicate with a cloud device 4002 which stores previously captured user information (e.g., from the user's personal device and/or other devices) in a central repository 4004. For example, the user is able to use his mobile phone to acquire facial recognition information, voice information, breath information, gait information, and/or any other information, and the acquired information is able to be uploaded to the central repository 4004 in the cloud device 4002 for comparison purposes. Additional information is also able to be acquired and stored for the user such as condition information including schedule information, eating habits, work information, and others. The additional information is able to help in the verification of the user's identity. For example, if the user typically goes to work at 8 a every weekday, and then a person appears at the work door entrance at 4 a on a weekend, it is not likely the user. Authorization information is also able to be stored in the cloud device 4002 or accessed by the cloud device 4002 to determine authorization. For example, identifying a user may be a first step in a process of providing authorization to open a door. A second step includes determining if the identified user is authorized to enter the door/building. Furthering the example, User A may be an employee at Company X, but does not have access to the lab, so while User A is identified by the device at the door to the lab, User A is not in the database of users with access to the lab, so User A will be denied access, but will be granted access to other offices/buildings of Company X where User A is in the database of users with general access.

Although a cloud device 4002 is described as storing the central repository 4004, the central repository 4004 is able to be stored across multiple devices. Although a single device as the device 4000 is shown, many devices are able to communicate with the cloud device 4002 to access the data of the central repository 4004. For example, instead of using keyed locks/doors, each door is able to be configured with a device 4000, and if a user is identified as having authorization to open the door, the device 4000 is able to unlock/open the door. This enables a user's identity data to be their roaming password. In other words, devices identify users, and based on their identification, they are able to be granted/denied access to devices/services/buildings.

Any of the implementations described herein are able to be used with any of the other implementations described herein. In some embodiments, the implementations described herein are implemented on a single device (e.g., user device, server, cloud device, backend device) and in some embodiments, the implementations are distributed across multiple devices, or a combination thereof.

Document signing systems have little or no facility to identify the person signing the document. Often, the identity is left to the recipient of the email or electronic correspondence. The outcome of this is that documents often need to be signed with ink (wet signatures), and the physical copy is mailed in directly. In more critical document signing situations, a notary public professional, who acts as a witness, records signatures in their presence. The function of the notary public is to manually guarantee the identity of the user signing the documents.

Two systems described herein are able to guarantee the identity of the signing party, account for the logging and accountability of the document at the time of signing, and guarantee the document is unmodifiable after the fact.

A unique technology is able to identify the user with several techniques. The user is identified by insisting the user provide official government issued physical identification (ID). The ID is scanned, recorded and saved as metadata for each document signature. The ID is analyzed exhaustively for any signs of tampering or counterfeiting. The ID is compared with government ID databases to guarantee the ID was issued to the specific user.

The user performs a live facial scan at the time of document signing. The scan uses several techniques to guarantee the scan is a live person, not a photo or digital copy: the user movements are identified, and the scan is a series of photos (or a video-like dataset). The live scan is compared to the picture on the government ID and is identified as the same user. The live image is attached as metadata to the document at the time of signing.

A multitude of human identity analytics are performed. Motion analytics and many other techniques are described herein as ID trust assurance. The identity analytics summary and quality score is attached as metadata to the document at the time of signing.

Other external data is collected at the time of signing and attached as metadata: time/date of every signature on the document, GPS/location data, other environmental data including weather, barometric pressures, satellite positions, and many others.

The document signing is performed by a smart personal device, such as a smartphone device. The document resides on a remote system and is presented on a local computer screen. There is a static or dynamic scan code next to each document signature field. There are often many codes and corresponding signature fields on a single document. The phone uses a camera (or potentially other sensors) to perform a signature. Simply scanning the dynamic scan code on the screen is able to constitute a signature. Each signature collects and records the real-time identity of the user. This is to guarantee the document was signed by only one person. This guarantees the signature process was not hijacked by another and signed by an inappropriate person. Optionally, the dynamic scan code is actually a streaming graphic, which helps ensure the document scan code cannot be copied and can only be scanned by the appropriate and identified user.

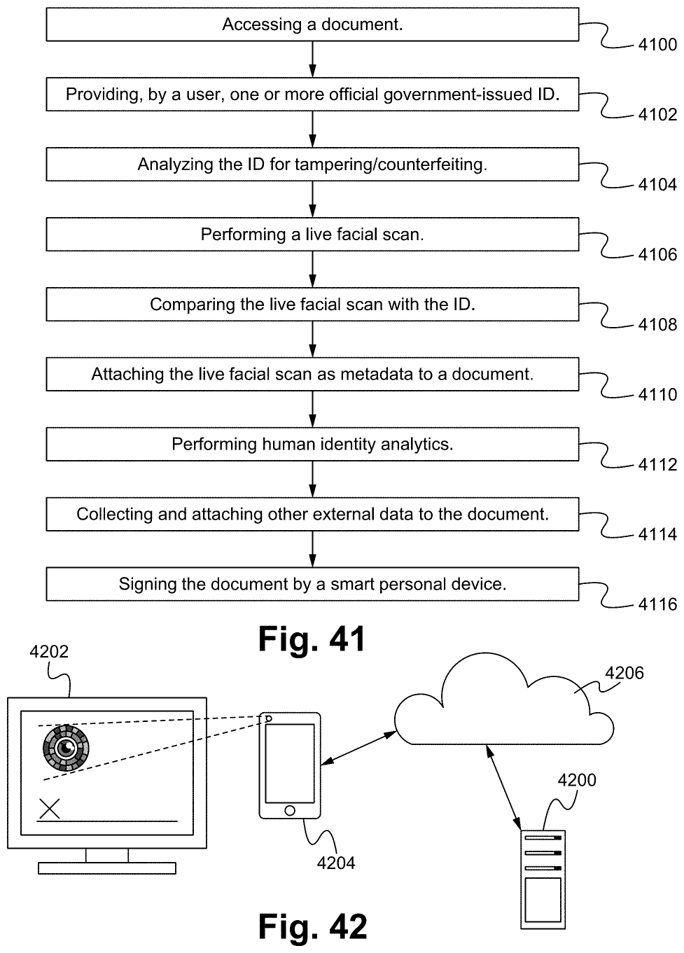

FIG. 41 illustrates a flowchart of a method of implementing document signing with the human as the password according to some embodiments. In the step 4100, a document is accessed for a user to sign. The document is able to be stored locally or remotely. For example, the document is stored in a cloud device, and is accessed by a computing device (e.g., personal computer). The document is able to be linked to/accompanied by metadata as described herein (e.g., trust score, environmental information, identification information, and more).

In the step 4102, a user provides one or more official government-issued IDs (e.g., driver license, passport). Depending on the implementation, the ID is able to be a physical version of the ID, a digital scan/copy of a physical version, or a digital version. The ID is able to be provided to a system/service by uploading the ID (or an image thereof) to a secure server. The ID is saved as metadata for each document signature. In some embodiments, other forms of ID are acceptable such as student IDs.

In the step 4104, the ID is analyzed for any signs of tampering or counterfeiting. For example, the ID is compared with government ID databases to guarantee the ID was issued to the specific user. The server is able to query government ID databases to determine if the ID or information on the ID matches stored government information.

In the step 4106, the user performs a live facial scan at the time of document signing. For example, the user holds the device with the camera facing the user so the camera is able to scan the user's face from multiple angles. The scan uses one or more techniques to guarantee the scan is a live person, not a photo or digital copy: the user movements are identified (e.g., by a comparison with previously stored movements using machine learning), and the scan is a series of photos (or a video-like dataset). In some embodiments, the user is given directions from the device in terms of which direction to look or move.

In the step 4108, the live scan is compared to the picture on the government ID and/or other identifying pictures. If the pictures match, then the user is identified as the same user in the ID. In some embodiments, although many pictures of the user are acquired to ensure the user is a live person and not a photograph, a single picture may be compared with the government ID. For example, pictures are taken with the user looking up, to the left, straight on, and to the lower right, but only the picture with the user looking straight on is compared with the government ID.

In the step 4110, the live scan (or an image of the live scan) is attached as metadata to the document at the time of signing.

In the step 4112, one or more human identity analytics are performed using a user device (e.g., mobile device). Motion analytics and many other techniques are described herein as ID trust assurance (e.g., the user's gait, biometric information, microtremors are analyzed). The identity analytics summary and quality (trust) score are attached as metadata to the document at the time of signing. For example, a description of the motions (or other user aspects) detected is attached as metadata. In some embodiments, the individual breakdown of how well each motion or biometric data matched is able to be included in the metadata. Also, for example, a trust score of 95% is attached as metadata.

In some embodiments, if the trust score (or other score) is below a threshold, the user may not be able to sign the document (e.g., the device will not sign the document). For example, if a user's trust score is 70%, but the threshold is 90%, then the user is not permitted to sign the document (e.g., scanning the scan code does nothing or signals a error/warning). In some embodiments, the threshold may depend on the type of document. For example, an unimportant document (e.g., agreeing to terms of use for website) may allow a user to sign if the user's trust score is at least 80%, but an important document (e.g., mortgage paperwork, change of name), may require a user's trust score to be 95% or higher to sign.

In the step 4114, other external data is collected at the time of signing and attached as metadata: time/date of every signature on the document, GPS/location data, other environmental data including weather, barometric pressures, satellite positions, and many others.

In the step 4116, the document signing is performed by a smart personal device, such as a smartphone device. The document resides on a remote system and is presented on a local computer screen. There is a static or dynamic scan code next to each document signature field. An example of a static scan code is a bar code, QR code or a static version of the eyeball described herein. The scan code includes document identification information such as (the document name/title and/or signature line). The phone uses a camera (or potentially other sensors) to perform a signature (e.g., the user holds the phone up to each scan code on the document for the phone to sign the document). Scanning the dynamic scan code on the screen is able to constitute a signature. In other words, the user does not need to type or sign anything using his finger; scanning the code is the signature since the device is recognized as the user. Once a scan code is scanned, the phone sends a signal to the local computing device and/or the server device storing the document to indicate that the specific signature line of the document corresponding to the scan code has been signed. In some embodiments, each scan/signature includes collecting and recording real-time identity information of the user. For example, the phone takes a picture of the user while the scan code is scanned (e.g., simultaneous pictures are taken using one camera facing a first direction and a second camera facing a different or opposite direction, and the scan is not triggered until the scan code is visible in one camera, and the user's face is visible in the other camera). This is to guarantee the document was signed by only one person. This guarantees the signature process was not hijacked by another and signed by an inappropriate person. In some embodiments, the dynamic scan code is actually a streaming graphic, which helps ensure the document scan code cannot be copied and can only be scanned by the appropriate and identified user.

In some embodiments, fewer or additional steps are implemented. For example, after the document is signed, the document is sent and/or stored in a remote location (e.g., the Cloud). In some embodiments, the order of the steps is modified. For example, the document is able to be accessed before or after the user provides government-issued ID. In some embodiments, if any of the steps fail (e.g., user does not provide a government-issued ID, user fails the live scan, user fails the human identity analytics, and so on), then the device does not sign the document. In some embodiments, the metadata or another implementation forms a shell around the document to fully provide a guarantee of the user's identity. For example, the document and the metadata are able to be encrypted and/or linked such that neither is accessible (e.g., cannot be read/opened) without the other.

FIG. 42 illustrates a diagram of a system for document signing with digital signatures with the human as the password. A server device 4200 via a network 4206 (e.g., the Internet) is configured to store and provide/share a document to be signed. Included with the document are one or more scan codes (e.g., one next to each signature line in the document). The scan codes are able to be static, dynamic or streamed. The server device 4200 (or another device) is able to be used to receive and analyzed the ID from the user. For example, the user uploads a picture of the ID to the server device 4200, and the server device 4200 compares the ID with stored ID information

A computing device 4202 accesses and displays the document including the scan codes. The computing device 4202 is able to be any computing device with a display such as a personal computer, a laptop, a tablet, or a smart phone.

A mobile device 4204 is used to scan the scan codes. For example, the mobile device 4204 is a smart phone with a camera which is able to scan the scan codes. The camera (or a second camera) is also able to take a picture of the user while the scanning occurs. Signing the document includes sending a signal to the computing device 4202 and/or the server 4200. The mobile device 4204 is also used to perform the live facial scan, and the human identity analytics. For example, the camera of the mobile device 4204 is used to perform a live facial scan of the user, and send the live facial scan (or one picture from the live facial scan) to the server 4200 (or another device), where the live facial scan is compared with the ID. In another example, the mobile device 4204 is used to perform the human identity analytics where the user holds the device, moves the device, moves with the device, and/or performs other biometric/behavior analytics as described herein. The mobile device 4204 is also able to collect and attach additional data to the document. For example, the mobile device 4204 sends time/date information, weather information, and/or other information at the time of the signature to the computing device 4202 and/or the server 4200. In some embodiments, the aspects described herein are performed on any of the devices or other devices.

In some embodiments, user identity based on human breath analytics is implemented. Every user has a unique breath pattern which can be monitored by personal or stationary devices. Analyzing a user's breath pattern is able to be performed by a smartphone device in personal possession by a user. The breath pattern is unique and can be monitored with a microphone and/or another device/sensor. Other sensors which can monitor breath include motion and heat sensors.

The breath pattern/information varies by many factors including: sound patterns, voice pattern techniques as described herein, breath pace, breath patterns (similar to heart beats), speed, depth, volume, and more.

The variations of breath will correlate with other human factors such as heartbeat pace. Breath qualities typically change based on other human conditions.

Breath pattern analytics are able to be passive, meaning this does not require any directed action for the user. Breath pattern analytics are able to be performed at the time of user transactions where the user will be close to and looking at the personal or stationary device. Breath pattern analytics are able to be performed in low-light or in pitch black conditions. Other analytics typically use light or direct physical possession (motion analytics).

Conditions with high levels of background noise could make this analytic of low effectiveness or invalid. However, there are ways of minimizing the background noise such that the breath pattern analytics are able to be used.

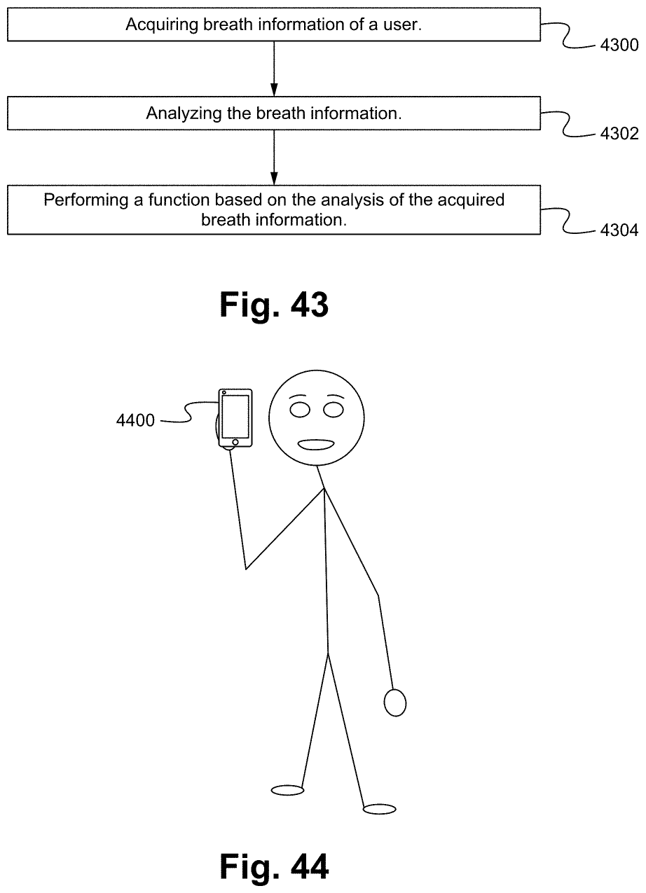

FIG. 43 illustrates a flowchart of a method of implementing breath pattern analytics according to some embodiments. In the step 4300, a device acquires breath or breathing information of a user. The device is able to be a user device such as a mobile phone, a stationary device or any other device. The breath information is able to be acquired using a microphone, a camera, and/or sensors of a device. For example, the microphone is able to record sounds of a user, and the camera is able to record breathing movements such as nostrils moving, chest/abdomen rising and falling, and a mouth opening and closing. In another example, a heat sensor is able to detect the heat of a user's breath, and when the sensor detects a warmer temperature, it is likely the user breathed out, and a cooler temperature would indicate the user breathing in, on a cold day. A hot day may involve a cooler temperature when a user breathes out, and a higher temperature when the user breathes in.

In the step 4302, the breath information is analyzed. The breath information is able to include one or more aspects/factors such as: sound patterns, voice pattern techniques as described herein, breath pace, breath patterns (similar to heart beats), speed, depth, volume, temperature, and more. For example, to detect sound patterns, a microphone of the user device detects sounds. In some embodiments, the sound is filtered or masked to eliminate any non-breath related information. Any sound processing is able to be implemented.

When users breathe, they may make distinctive sounds such as their nose whistling, their throat making a sound, or their lips causing a whistle. Similarly, an open-mouthed breath sounds different than when one breathes through his nose. The sounds are able to be detected when analyzing the acquired sound information by machine learning and comparing the sound information with previously stored information.

Sound patterns are able to be detected based on the analysis of the sounds detected. For example, after analyzing sound patterns for a length of time, specific patterns may be detected such as detecting that a user's nose typically whistles two seconds after the user takes a breath in. Any pattern detection/matching is able to be detected. In another example, it is detected that a user makes a grunting sound before breathing in.

Similarly, users have specific breathing patterns when they talk. For example, some users may take deep breaths before or after talking, while other users may only take short breaths. The length of a breath is able to be measured using specific starting points (e.g., sounds of a breath blowing out) and end points (e.g., the breath stopping). Machine learning is able to be used to detect patterns in a user's breath or breathing.

Breathing patterns are able to be detected at other times as well (e.g., when the user is not talking). Breath(ing) patterns are able to be detected by measuring a duration between each breath (e.g., start to start or end to end), the volume of each breath (e.g., in decibels), and detecting the duration and volume over a period of time (e.g., 5 s, 30 s) to determine a pattern similar to a heart beat. Detecting a breath is able to be performed by sound matching (e.g., machine learning learns what a breath sound is. Moreover, each aspect of a breath is able to be detected. For example, a breath in makes a different sound than a breath out, and each is able to be detected. Similarly, there are pauses between each breath, where the amount of time of the pause is able to be slightly different for each user.

Breath pace/speed is another distinction of users. For example, some people take long, deep breaths while others take short breaths. The time interval between each breath and/or the duration of each breath is able to be computed. For example, the time in between detecting a breath in and the next breath in is able to be calculated. In another example, the duration that a user breathes in until a breath out is detected is able to be calculated. The volume of a breath is also able to be detected. For example, the volume of a user's breath is able to be measured in decibels for comparison purposes.

Distinct/unique breaths are also able to be detected. For example, if a user every once in a while takes a unique breath (e.g., quick breath with a crackle) due to an illness, anxiety, or any other reason, the unique breath is able to help distinguish the user's identity. The unique breath sound is able to be stored, and if the user makes the same unique breath again at a later date, then the user is likely able to be identified as the same user.

Breaths are able to be detected using heat/temperature sensors which are able to indicate when a user is taking a breath or is blowing out. In one example, if a user's breath is typically around 98 degrees, then each time, a temperature sensor detects 98 degrees, a user has likely breathed, and a time between each detection is able to indicate the duration between each breath. For more accuracy (e.g., in case the ambient temperature is around 98 degrees), a motion/wind sensor is able to be used to detect the movement of the air from the breath in addition to the temperature sensor detecting the temperature of the breath.

Any of these breath characteristics are able to change based on condition information. For example, a user's breath is able to be affected by weather, exercise, stress/anxiety, altitude, location, and many other factors.

Analysis of the breath information is able to include comparing the current breath information with previously acquired information. For example, a device initially uses other analytics to identify a user and enable access based on identifying the user. During a specified time period, the device acquires and analyzes a user's breath information. Then, when an adequate amount of information has been acquired and analyzed, the stored breath information is able to be used for comparison purposes with currently acquired user breath information.

As described herein for other analytics, the breath information is able to be grouped/classified based on condition information. For example, a user's breathing is likely to be very different when at rest when compared with during or after running. Similarly, a user's breathing may be different when the user is in a cold environment versus a hot environment. The breath information is able to be classified based on the condition information by using any other analytic information to determine if any condition information is relevant for classification. Similarly, when new/current breath information is acquired, the condition information is able to be used to compare the appropriate stored breath information. A comparison of breath information of a user currently sitting at his desk with breath information while the user is running will likely result in no match; however, a comparison with breath information of the user at rest is likely a match. In some embodiments, if different condition classifications are generated, and later it is determined that the breath information is the same for the different classifications, then the classifications are able to be merged/joined or linked such that the same or very similar breath information is stored only once instead of twice. Analysis of the breath information is able to include video analysis. For example, a camera of a mobile device is able to acquire the movements of a user's nostrils, chest, abdomen, throat, mouth and/or other body parts to determine a user's breath information such as starting a breath, ending a breath, the duration of the breath, any specific characteristics of the breath, and so on.

In the step 4304, a function is performed based on the analysis of the acquired breath information. For example, a user's trust score is adjusted based on the breath analysis. If the comparison of the user's current breath information matches the stored breath information, then the user's trust score is able to be maintained or increased as described herein. If there is not a match, then the user's trust score is able to be decreased as described herein.

In some embodiments, fewer or additional steps are implemented. In some embodiments, the order of the steps is modified.

FIG. 44 illustrates a diagram of performing breath pattern analytics according to some embodiments. The user is able to hold a mobile device 4400 (e.g., a smart phone) and talk as the user typically would. The microphone, camera, and/or sensors of the mobile device 4400 are able to detect and capture the user's breath information. In some embodiments, the mobile device 4400 processes the breath information using the processor and memory of the device. Processing is able to include sound/signal processing such as using filters, masks and machine learning to determine specific breath information among other sound information. The processed information is able to be compared with stored breath information to determine if the currently acquired information is a match of previously stored information. A match would indicate that the user is the authorized user, and no match would indicate that the user is not the authorized user. In some embodiments, the breath information is sent to a remote device (e.g., a server in the Cloud) for processing (e.g., analysis and/or comparison).

Many of the analytics described herein involve ongoing analytics which are background scheduled or run continuously. For many personal devices, this causes issues with security, system performance and other management issues. In some cases, operating environments do not support background processes and require foreground applications for active user interactions.

A class of human and device analytics are described herein which run in the foreground when a transaction or authentication app is initiated and running interactively.

Prior to performing an activity, such as starting a new process or initiating a specific transaction, there are passive analytics and/or active challenges presented.

Passive analytics do not require user interactions requested by the system. One or more ad-hoc analytics are performed prior to allowing the initiation of the action. Analytics performed in this class can quickly and accurately identify the specific user. Examples of these analytics include: live face recognition—since the user is probably staring at the personal or stationary access device, the face will likely be available to the built-in device camera; voice pattern and quality analytics; breath pattern and quality analysis; external factors including location patterns, user height, environmental and weather; and micro-motion analytics.

Active challenges are able to be utilized when the system cannot passively identify the current user accurately. The system requests the user to perform an action. This action should both identify that the current user is an actual human and can only be performed by the specific user. For example, challenges include: the shake challenge, a live 3D facial scan, and a directed voice print and quality analysis.

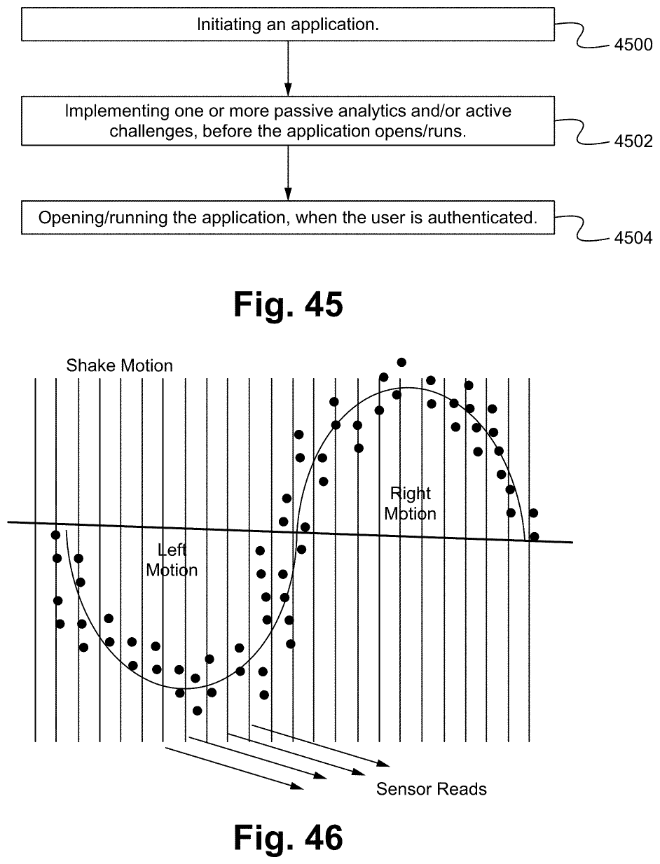

FIG. 45 illustrates a flowchart of a method of performing passive analytics or active challenges prior to starting a new process or initiating a specific transaction according to some embodiments. In the step 4500, a user initiates an application. Initiating an application is able to include selecting an online application link, single or double-clicking an application icon, voice-activation and/or any other implementation of initiating/triggering an application.

In the step 4502, before the application opens/runs, one or more passive analytics and/or active challenges are implemented. In some embodiments, the application opens/runs to the point of triggering the one or more passive analytics and/or active challenges, but the other aspects of the application are not opened until the user is authenticated. For example, if the user selects a word processing application, the application opens to allow the passive analytics/challenges to be performed, but the word processing aspects remain unavailable to the user. As described herein, the passive analytics are able to include live face recognition, voice pattern and quality analytics, breath pattern and quality analysis, and others. For example, using the camera of the device, a user's face is scanned, and when the user's face matches stored facial information, the user is authenticated. Active challenges include the shake challenge, a live 3D facial scan, a directed voice print and quality analysis, and others. For example, if the user is wearing a face mask, and the facial recognition does not authenticate the user, then the user is presented a challenge to shake the device. If the user's shaking pattern matches stored shake information, then the user is authenticated. In some embodiments, external factors are analyzed and accounted for when performing the passive analytics and active challenges. The external factors are able to include location patterns, user height, environmental and weather conditions, micro-motion analytics, and others. As described herein, the passive analytics and/or active challenges may be affected by the external factors. Categorizing the user information and acquired information enables more accurate comparisons and authentication results.

In the step 4504, when the user is authenticated based on the passive analytics and/or the active challenges, the application opens/runs. For example, if the user selects a word processing application, the word processing application opens (e.g., enables access to word processing features) when the user is authenticated. If the user is not authenticated (e.g., fails the passive analytics and active challenges), then the application will not open (e.g., the application shuts down without providing access to the word processing features). In some embodiments, authenticating the user involves affecting a trust score, and when the trust score is above a threshold, the application opens/runs. In some embodiments, the application opens/runs with limited accessibility/options when the user's trust score is above a first threshold but below a second threshold. For example, Application X has a user trust score threshold of 75 to open/run, but the features to purchase items via Application X are not accessible when the user trust score is below a second threshold of 90. When the user's trust score is above 90, the application/features would be fully accessible go the user. In some embodiments, the order of the steps is modified. In some embodiments, fewer or additional steps are implemented.

In some embodiments, the passive analytics and/or active challenge features are embedded within each application which provides additional functionality (e.g., word processing, digital payments, social networking, image editing, and many others). In some embodiments, the passive analytics and/or active challenge features are bundled/batched with each application that provides additional functionality. For example, the analytics/challenges are a separate application which are coupled with a word processing application, and when the user selects the word processing application, the analytics/challenges application opens initially, and when the user is authenticated, the word processing application is triggered to open. In some embodiments, the passive analytics and/or active challenge features are implemented as background applications, which monitor when an application is selected and are initiated when the application is selected. The application does not become fully accessible until the user is authenticated.

Most machine learning algorithms are based on collecting a large collection of data and feeding the data into a set of algorithms such as data point cluster analysis, and can identify repeating patterns in a data set.

As new data is available, this large data set is again batched and a new machine learning model is generated. Having to generate a new learning model causes a large overhead for computer resources.

A modified version of machine learning works continuously on incoming data and performs analytics as each data record arrives. The continuous calculations use very little computer overhead. This is an important aspect for small devices such as smart phones, personal computers and IoT devices.

The specific example provided herein is analysis of human motions, but the modified version of machine learning is more general and is able to be applied to other use cases. The form of continuous analytics provides mechanisms for cases such as: behavioral analytics, presenting various methodologies for calculating human id trust. Methodologies include movement analysis, location pattern anomalies, gait characteristics (step length, pace, speed, and others).

One example of a form of motion analytics is the device shake motion, where a user moves a device up and down and/or back and forth. The “shake” motion is an acceleration followed by deacceleration in one direction, and the same for the return motion. Gyroscope sensors are also involved which monitor and collect circular momentum motions. Other motions/movements are also able to be monitored, detected and recorded.

The application samples the movement sensors during the shake movements generating data points for each sensor. If the data points of a single sensor are plotted on a graph, the resultant curve resembles a standard sine-like wave as shown in FIG. 46. The patterns will consolidate around a statistical curve (or baseline in this context).

The modified machine learning system typically has a learning period, where a set of data is input, and when an adequate number of records are collected and computed, the baseline is a valid representation of standard pattern.

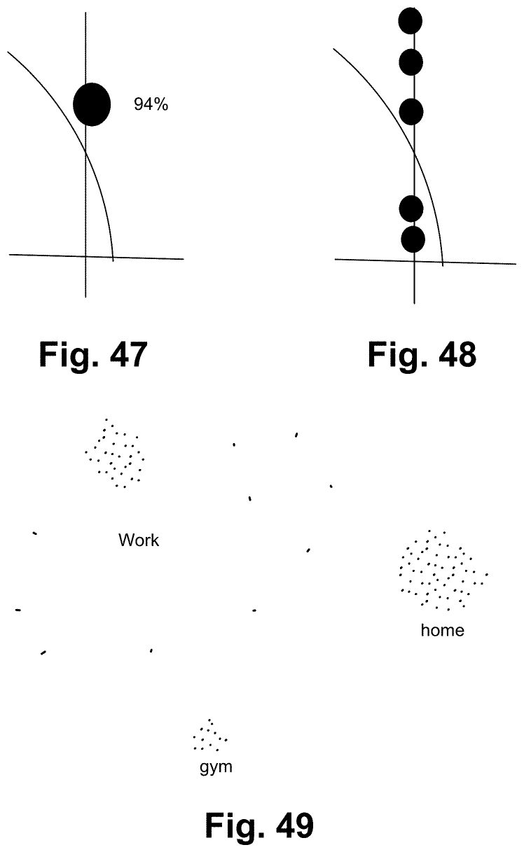

FIG. 47 illustrates a diagram of a set of data points to be used to calculate a baseline according to some embodiments. The baseline is generated by repeating the shaking motion. Each of the shake sensor data points sampled will cluster around a pattern. This generally varies during each shake. With each shake sampled, the accuracy and statistical deviation accuracies will converge.

There are able to be multiple clusters. For example, a separate shake motion might occur when the user is drinking heavily, or tired, or when traveling in a vehicle.

The baseline for each shake motion is computed separately for each of the 6 axes.

FIG. 48 illustrates a diagram of a calculated baseline according to some embodiments. In the example, the shake motions deviated from the baseline curve slightly, which resulted in a trust score of 94%.

The calculation for each axis is:

Trust=Σ_{C}¹(S)1/C
where C=total count of shake motions in the data set,
and S=the complete set of sensor readings.

To avoid the overhead of storing the large data set of motion sensor readings and recalculating using the complete set, the calculation is able to be performed for each sensor reading for each polling interval. The resultant is stored in a baseline array in the local database. The final trust score is derived using the calculated deviation from the baseline value of each axis value, then averaging the deviation of all 6 axes.

### Example:

Baseline array readings for 10 data points (e.g., of acceleration) for a single shake motion:
S [Motion Data Points] [1.0, 1.3, −0.5, 1.1, 3.1, −0.1, −0.3, 0.01, 0.5, 0.0]
Baseline Data Points [1.0, 1.3, −0.5, 1.1, 3.1, −0.1, −0.3, 0.01, 0.5, 0.0]
C [Count]=10
In this example, the accuracy is 100%.
The new baseline would remain the same but the Count value would become 11.

A new activity or data input is able to be compared to the baseline data sets to determine if the current activity matches. These matches are able to be used to identify various details, such as matching a human motion with a learned baseline; matching other patterns such as locations or other reoccurring incidents (e.g., location, speed or movement patterns); temporal patterns; voice patterns and qualities, and others.

FIG. 49 illustrates a diagram of clusters of data points of location information according to some embodiments. There are clusters of data points for locations the user frequents most often such as home, work and the gym. Other data points are able to be found scattered about based on where the user has been. The clusters are usable information in terms of helping to identify the user. For example, if it is determined that the user is located in a cluster, it is more likely that the user is the authorized user (e.g., trust score is increased by 3 points). In another example, if analysis determines that the user moves from one cluster to another cluster, then the likelihood that the user is the authorized user is even higher (e.g., trust score is increased by 5 points). The number of clusters the user visits within a specified amount of time is able to increase the trust score. Moreover, when the user is not at a cluster, the trust score is able to stay the same or decrease (e.g., by 5 points). In some embodiments, the location cluster information is able to be used in conjunction with other information to affect the trust score.

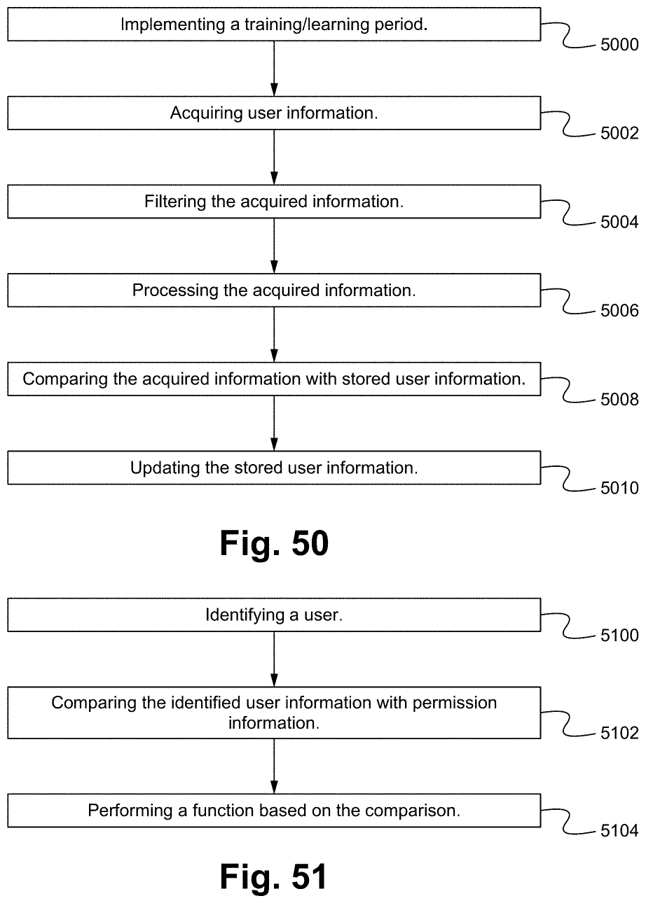

FIG. 50 illustrates a flowchart of a method of implementing a modified version of machine learning according to some embodiments. In the step 5000, a training/learning period is implemented to acquire user information. During the training/learning period, a device (e.g., mobile phone) acquires a set of data until an adequate number of records are collected and computed. The number of records is able to programmed by a developer (e.g., do the training 10 times) or until a sufficiently clear pattern has been determined. A template or other statistical analysis is able to be used to determine whether a pattern has been developed (e.g., if 5 data sets are within a specified numerical range of each other, then the pattern is considered clear. Upon collecting an adequate number of records, a baseline representation of the data is generated. Different baselines are generated for each action/motion/feature (e.g., the baseline information is separated into categories). Additionally, different baselines are able to be generated when there is conditional information (e.g., sub-categories of baseline information are generated). For example, a shake motion when is a user is at rest is able to be different from a shake motion when the user is drunk which is able to be different from a shake motion when the user is driving. Additionally, depending on the type of activity, there may be multiple sets of information gathered (e.g., for shake motion, there are 6 axes of information), and a baseline is computed for each of the 6 axes. In some embodiments, the training includes line fitting and/or clustering after some filtering is performed.

In the step 5002, after the training period, user information is acquired by the device (e.g., current user information or newly acquired user information), as described herein (e.g., acquiring user motions, user body parts, user voice, and more). The user information is able to be acquired passively or actively. For example, the user device is able to acquire the information without the user being told to perform a specific action (e.g., acquiring voice or breath information while the user is talking or gait information while the user is walking). In another example, the user device is able to direct the user to perform an action (e.g., shake challenge).

In the step 5004, the acquired user information is filtered. For example, if acquired user information does not meet specified criteria, then the user information is discarded. Furthering the example, if the user information is not within a similarity range of the previously acquired information, then the user information is discarded. Continuing with the example, if gait analysis is being performed, but the user is actually dancing, then the movement information of the dance will be discarded. Determining whether the user information is within a similarity range is able to performed by comparing data points and/or cluster information of stored information and acquired information.

In the step 5006, the acquired data is processed. In some embodiments, the acquired data is processed by an algorithm such as clustering and/or line fitting. For example, the algorithm determines clustered data points within the acquired data. In another example, the algorithm performs line fitting to construct a straight line that has the best fit to a series of data points.

In the step 5008, the currently acquired user information is compared with the stored user information. For motion, the comparison is performed for each of the 6 axes (e.g., a sine wave or other wave/pattern/signal for each of these axes). For location (e.g., GPS), there will be clusters of locations that the user frequents (e.g., home, work, gym). Other types of activities may result in different types of analytical structures. The comparison involves comparing the current data set (e.g., the data set of the currently acquired information) with the baseline information. For example, an acceleration value of a data point at a specific time is compared with a baseline data point in terms of acceleration and time, and the amount that they are different is determined. Each data point difference is combined to generate the collective difference. When the current data set is different from the baseline information, the trust score for the analytic is affected. For example, if the current data set matches the baseline information exactly, then the trust score is 100%. However, if there are subtle differences, the trust score may be 94% or another number (e.g., each subtle difference is added together to determine a total difference). If there are major differences, the trust score may be 25%, 50% or another relatively low number. To avoid the overhead of storing the large data set of motion sensor readings and recalculating using the complete set, the calculation is able to be performed for each sensor reading for each polling interval, and the resultant is stored in a baseline array in a local database. A final trust score is determined using the calculated deviation from the baseline value of each axis value, and then averaging the deviation of all 6 axes. Other calculations are able to be implemented in some embodiments.

In the step 5010, the stored user information (e.g., baseline) is updated based on the currently acquired user information. In some embodiments, the stored user information is updated each time new user information is acquired. For example, over time, a user's gait or other motion/feature may change, so the baseline information (stored information) is able to be updated continuously. In some embodiments, the stored information is only updated when the comparison of the current information with the stored/baseline information is above a threshold. For example, if the currently/newly acquired information results in a trust score of 25% (with the threshold of 80%), then the currently acquired information is not stored to affect the stored/baseline information. Other conditions are able to be applied for when the baseline information is updated. For example, if the currently acquired user information is not close (e.g., not above a threshold), but the user is authenticated in another manner, the baseline information may be updated with the currently acquired information. In some embodiments, the baseline information is only updated when a user enters a re-training mode by first authenticating the user, and then performing training again to supplement or replace the baseline information.

In some embodiments, fewer or additional steps are implemented. In some embodiments, the order of the steps is modified. For example, when the trust score of the user of the device is above a threshold, then the user is able to access a service or device (e.g., a second device).

In addition to being used for human-related machine learning, the modified machine learning is able to be utilized for non-human behavior such as detecting device status and operation. For example, battery performance, app performance, processor performance, and other aspects of a device are able to be compared with a baseline to detect anomalies. Furthering the example, as the processor runs, certain data is able to be collected and analyzed to generate a baseline such as based on the percent usage of the processor depending on which applications are running. If the same applications are detected as running, but a higher percent usage of the processor is detected, it may be based on malware utilizing processor bandwidth. An alert or other action is able to be taken when non-user-based information is detected as unmatching (or beyond a threshold away from) a baseline. Similar to the modified human-related machine learning, non-human-related machine learning involves acquiring information, generating a baseline based on the information (where the information is able to be discarded once the baseline has been generated), and then continuously adjusting the baseline based on newly acquired information. The baseline is able to then be used to detect issues such as a faulty/failing battery, malware/virus, a problematic application, and/or any other device-related issue. The comparison of the newly acquired information and the baseline is able to be used to provide a trust score of the device which is able to be used to trigger an action such as an alert.

This invention extends the functionality of the Patent Disclosure where user identities can be determined by on-premises or personal devices. Users can be identified by appearances, movement analysis, voice patterns and many other factors. Since a user can be identified by on-premises systems, the user's behaviors can be monitored for unauthorized area access, unsafe behaviors or suspicious behaviors.

Each user is able to have a level of permitted access or behaviors, and the access/behaviors are able to be related to a user's identity and an organization's policies. The technology described herein is able to be used to implement “smart alarms,” “security policy monitoring” and others.

FIG. 51 illustrates a flowchart of a method of implementing user movement and behavior tracking for security and suspicious activities according to some embodiments. In the step 5100, a user is identified. As described herein, a user is able to be identified using passive analytics and/or active challenges. The user is able to be identified using his device, a stationary device and/or another device. For example, the user's device identifies the user based on the user's gait, breath pattern, and a shake challenge. In another example, a stationary device identifies the user based on the user's gate and voice recognition. In yet another example, a user device (e.g., smart watch) detects the user's heart rate pattern, and the stationary device detects the user's gait, and based on analysis of the heart rate pattern and gait, the user is identified.

Identifying the user includes acquiring user information in any manner such as acquiring image/video information using a camera, acquiring audio information using a microphone and/or acquire other information using other sensors.

The acquired user information is analyzed/processed to identify the user. Analyzing/processing the information is able to include image processing, video processing, audio processing and/or any other processing to separate and classify the different aspects of the acquired information. Further processing is able to take place to analyze each aspect (e.g., processing the leg and arm motions to determining specific characteristics of the user's gait). Additionally, if there is condition information that accompanies the user information, the condition information is able to be acquired as well or is able to be used to further classify the user information. Analyzing/processing is also able to include machine learning or the modified machine learning described herein to perform pattern matching and/or other analysis and comparisons of information.

Analyzing/processing the acquired information is able to include comparing the acquired information with stored information (e.g., from previous training and/or other stored information). In some embodiments, the stored information is stored locally and/or in a central repository accessible by the device (and other devices such as Internet-connected devices). In some embodiments, the stored information and the acquired information are stored such that the underlying data is unreadable (e.g., hashes of the information are stored and compared). Any form of securing the data, but also allowing the data to be compared is able to be implemented.

The comparison is able to include many aspects/details of the acquired information. For example, biometric recognition, voice recognition, motion analysis, gait analysis, breath analysis and other analysis are able to be implemented. As described herein, machine learning or the modified machine learning described herein are able to be used to perform the comparison. Each separate aspect of the acquired information is able to be used for comparing with the stored information, and each separate aspect is able to receive an identification score and/or a pass/fail. By performing multiple forms of analysis, the chances of tricking a system are decreased, and the confidence of accurately identifying a user is able to be increased. Any implementation is able to be used to determine whether the user is identified.

In the step 5102, the identified user information (e.g., a unique user identifier) is compared with permission information. The permission information is able to be structured in any manner such as a hierarchical structure where those at the top of the hierarchy have more access than those at the bottom. For example, a CEO of a company has access to all locations (e.g., doors/buildings) of a company, while a cafeteria worker may only have access to the cafeteria door/building. The permission information is able to be stored locally and/or remotely. The permission information is able to be compared with the identified user information using any search/look up implementation (e.g., a database search to compare names). Upon finding a match, the stored permission information is linked to/corresponds with access information. For example, if the identified user is User A, and User A is located in the permission information (e.g., an access database), then that name is also linked to access to Doors 1, 2 and 5, but not Doors 3 and 4. The permission information is able to be stored in separate storage structures or a single storage structure. For example, each device (e.g., door, phone, building) has a corresponding storage structure with permission information. In another example, a user's name (or other identification information) includes permitted access locations (e.g., front door of home, back door of home, doors 1, 2 and 3 of work). In some embodiments, prohibited information is also stored. For example, if User A is prohibited from a school zone, then that prohibition information is able to be linked to the user's identification information.

In the step 5104, a function is performed based on the comparison. If the comparison results in a match (e.g., the identified user has access permission to a specific device/location), then an action is triggered such as unlocking the door. If the comparison does not result in a match, then an action such as triggering an alarm or no action occurs (e.g., door remains locked). Other functions performed are able to include triggering an alarm, sending a message, taking a picture/video, and/or any other function or action. In some embodiments, performing the function includes sending a signal to another device for the other device to perform an additional function. For example, if a user's mobile device identifies the user and determines that the user has permission to open a door, then the mobile device sends an unlock signal to the door or sends a permission signal which a door device receives and then unlocks the door.

In an implementation where a user is on a prohibited list, if a match is found, a function is performed/triggered such as triggering an alarm.

In some embodiments, fewer or additional steps are implemented. In some embodiments, the order of the steps is modified.

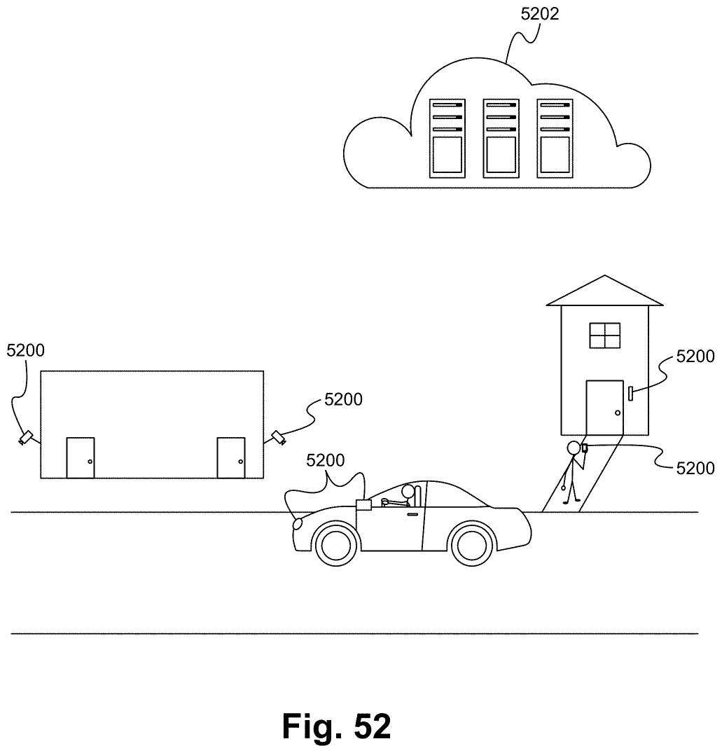

FIG. 52 illustrates a diagram of a system implementing user movement and behavior tracking for security and suspicious activities according to some embodiments. Devices 5200 are configured to acquire user information. As described, the devices 5200 are able to include a camera, a microphone, sensors, and/or other components to acquire the user information. The devices 5200 are able to include user devices (e.g., smart phone, smart watch, tablet, personal computer), stationary devices (e.g., camera doorbell, security camera, wall-mounted device), vehicular devices (e.g., autonomous vehicle), and/or any other devices capable of acquiring user information and/or conditional information. In some embodiments, one or more of the devices 5200 utilize ultrasonic waves for detection and/or identification of users. The devices 5200 are able to communicate with each other directly and/or through a network/cloud device 5202. The implementations described herein are able to be processed locally (e.g., on one of the devices 5200), remotely (e.g., in the cloud device 5202), and/or a combination thereof. Similarly, stored user information (e.g., based on training information), newly acquired user information, and/or permission information are able to be stored locally and/or remotely. The identification of the user is able to occur based on the local analysis and/or remote analysis. For example, a user is able to be identified by his gait using his mobile phone, and the mobile phone is able to communicate identification information to a security system to enable the user to open the door. In another example, a user is identified by his gait via a security camera, and the security camera is coupled to a security system which is able to allow the user to open the door. In yet another example, a user is partially identified (e.g., not with enough confidence to be considered identified) based on his gait, so a security system asks the user to perform a voice identification challenge by speaking into a microphone of the security system and a shake challenge by shaking his mobile phone to fully identify the user. The devices 5200 are able to acquire, store, transmit and/or process additional information such as condition information including schedule information, eating habits, work information, and others. The additional information is able to help in the verification of the user's identity. The cloud device 5202 is able to include multiple devices (e.g., many server and storage devices spread throughout the world).

The following are exemplary implementations of the user movement and behavior tracking for security and suspicious activities method/system.

When a user is near or touching a bank vault, but that user is unauthorized to do so, an alert is able to be triggered. The user is able to be identified using a security camera and/or a signal sent from the user's smart phone, the identification of the identified user is able to be compared with a bank list of identifications of authorized users for the vault, and the security system is able to trigger the alarm. Additionally, the security camera or other motion/touch/proximity sensors are able to detect that the user is within a specified range of the vault.

When a person enters a construction area without proper attire such as a hard hat, an alert is able to be triggered. Cameras in the construction area are able to be used to monitor for vandalism and theft, but also to perform image/video analysis to determine the attire being worn by a person in or entering the area. The image/video analysis is able to be used to perform template matching or other analysis to determine if the person is wearing a hard hat and/or other protective gear.

If a person under the influence (e.g., drunk, high) attempts to operate a vehicle (e.g., car, crane), then an alert is able to be triggered, the vehicle is able to automatically shut off or operate in autonomous mode, and/or authorities are able to be contacted. For example, based on the user's hand movements, a shake challenge, stumbling gait, and/or other detection/analysis, a device (e.g., mobile device, vehicle device) determines the person is under the influence, and the vehicle automatically shuts off.

If a person is another person's bedroom (e.g., looking in their underwear drawer), the camera on the user's television or computer is able to detect and identify the person. If the person is not identified as an owner/resident of the house (or possibly specifically even the resident of the room), then the security alarm is able to be triggered and/or a text message is able to be sent to the resident of the house/room.

A system is able to continuously monitor for authorized and unauthorized people inside and/or entering buildings. The system is able to passively or actively ensure that only authorized people are on-premises and can alert security for unauthorized access. In an implementation, a home alarm is always on, and this allows the family to come and go without disarming the alarm, as the alarm is only triggered when an unauthorized person enters or is inside the home. The door to the home is automatically locked (and the alarm is armed), but when an authorized user approaches, the door automatically unlocks (and the alarm is temporarily disarmed), but then re-locks (and re-arms) after the person enters. Cameras/devices on the inside of the house enable people to easily exit the house in a similar manner. In some embodiments, a user's mobile device communicates with a door device for authorization. A more refined implementation is able to permit authorized guests to enter the home (e.g., friends/service people) by temporarily adding a person or people to an authorized list. The person is able to be added to the list with a set period of time, and then the person's identification is able to be automatically deleted from the list after that period of time expires. Further refinement is able to be implemented such as a guest being on the list as long as the guest (e.g., child's friend) is accompanied by a person who is on the authorized list (e.g., child). The system is able to have an ongoing memory of the users detected/identified within the house.

Since the user movement and behavior tracking for security and suspicious activities method/system is able to identify users exactly, the method/system is able to provide additional features including: tracking the attendance or participation in meetings (e.g., each person attending is able to be related to projects and individual or project performance); tracking the locations of the employees; cross referencing the skills of certain employees with their success in customer sales performances (this is able to determine successful teams and project tasks); tracking data and identity data are able to be fed into a big-data data lake and used for data analysis; and analyzing employee performance within the company over a long period of time to understand the conditions certain employees perform better in.

A bedside user device advances the ability of a smart personal device to analyze a user's breath pattern using sensors embedded in the device including microphones, extremely accurate motion sensors and/or other sensors/devices. From this breath pattern, the following benefits are able to be obtained: sleep analysis, sleep pathologies, and environmental conditions.

Sleep analysis includes analyzing different aspects of a user's sleep. Sleep includes a variety of modes including light sleep, deep sleep, waking state, bodily movements including tossing about, involuntary movements like leg kicks and a number of other factors related to human sleep patterns. Sleep analysis is able to also monitor the partner of the user in order to understand how each partner's sleep patterns are impacting the other. Sleep analysis is able to include monitoring breath/breathing patterns while sleeping, snoring patterns, talking/laughing in sleep patterns, movement patterns, and/or other analysis.

Sleep pathologies are abnormal conditions which are able to occur during sleep including excessive snoring, sleep apnea and various other conditions and problems.

Environmental conditions are external factors which are also able to be monitored and recorded by the personal device including temperature, background noises, and more. Background noises are able to be further identified and categorized such as televisions playing during sleep, other people or animals present, noises (e.g., traffic noise), and activities noticeable to the sleeping user. The external factors potentially affecting sleep can be analyzed and correlated to sleep quality.

Other uses of the personal device may include monitoring user movement activities during non-sleep times. These activities include: movement performance such as walking, typing, reflex performances and other motion analytics which can provide energy and performance metrics for the user; overall amount of user movements and motions; impact of certain physical ailments on movement and sleep; and an eating schedule. The movement analytics are able to determine individual user energy and health levels when measured over time. Reduced movements or movement performance are able to be accurately measured. With these and other factors monitored and recorded, sleep quality is able to be correlated to daily energy levels and motion performance.

In addition, breath pattern analysis and behavioral qualities such as movement analysis are also able to be used to determine the effect of people using the bedside user device, which is able to allow a deeper understanding of their moods and how certain events, or substances impact their performance.

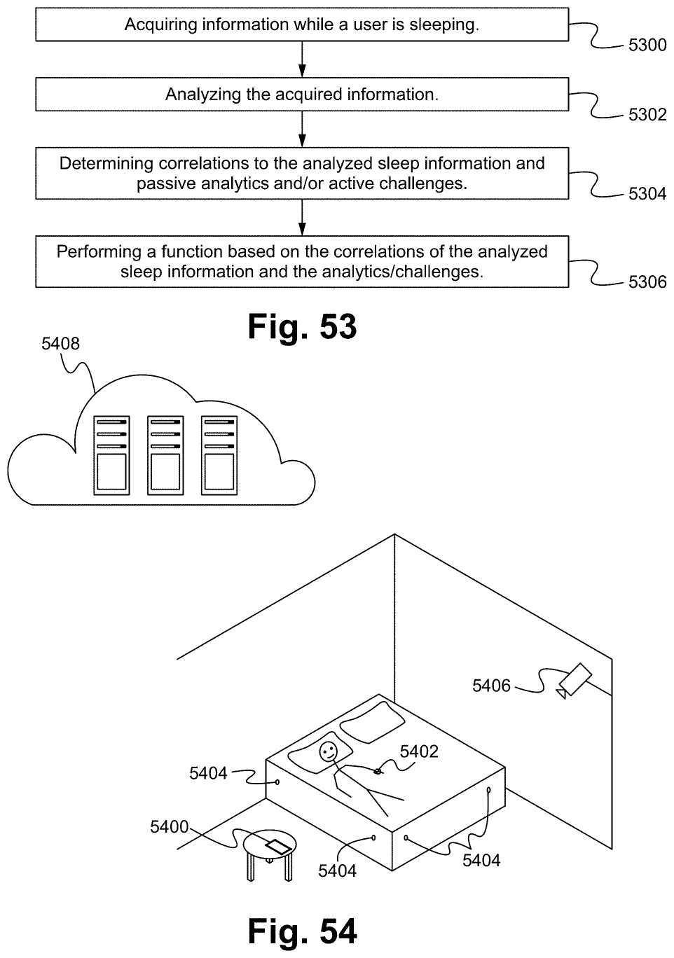

FIG. 53 illustrates a flowchart of a method of implementing a bedside user device according to some embodiments. In the step 5300, information is acquired while a user is sleeping. The information is able to be acquired by a user device (e.g., mobile phone), a stationary device, and/or another device (e.g., a device within a bed). The information is able to include audio, video, sensor information, and/or any other information. The information is able to be acquired using a microphone of the device (or other devices). For example, the microphone is able to detect breath sounds, vocal sounds, snoring, bodily movement sounds, ambient sounds (e.g., noise from traffic, televisions, animals, other people) and/or other audio. The information is able to be acquired using a camera of the device (or other devices). For example, the camera is able to positioned to monitor the user in bed and detect movements of the user. The information is able to be acquired using sensors of the device (or other devices). For example, a mobile phone is able to be positioned on a bed, and using motion sensors of the phone, movement of the user in bed is able to be detected. The sensors are able to acquire other information as well such as temperature, light (e.g., overall amount of light in the room or specific light sources), humidity, and/or other information. A wearable device is able to acquire physical information which is able to be associated with sleeping conditions (e.g., a slower heartrate and lower body temperature may be associated with light sleep). In some embodiments, one or more devices communicate information to each other or to a central/network device (e.g., a mobile device, security system cameras and bed sensors work together to acquire audio, video and sensor information of the user while sleeping). In some embodiments, a breathing device such as a CPAP device is configured to communicate with other devices (e.g., a user device) to provide details about the user (e.g., breath information).

In the step 5302, the acquired information is analyzed. Sound information is able to be analyzed to detect the types of sounds, quantities of the sounds, patterns of the sounds, and/or other sound analysis. Any audio/signal processing is able to be implemented such as template matching, decibel detection, and/or others. In some embodiments, machine learning or the modified machine learning described herein is used for the analysis. For example, each time a sound is detected, the sound is compared with a template or otherwise classified using machine learning such that a snore is classified in a snoring category, while other sounds are classified accordingly such as breathing, movement sounds, talking, moaning, grunting, screaming, television sound, another person talking, traffic, and many more. Each time a sound is made, a timestamp is able to be recorded in addition to the duration of the sound, the loudness of the sound, how often the sound occurs, and/or any other characteristics of the sound. Patterns of the sound are able to be detected. For example, a user typically snores in a relatively rhythmic manner, so a snoring pattern is able to be determined. Interruptions or anomalies to patterns are also able to be detected. Corresponding and/or causal relationships to sounds are able to be detected/determined. For example, if a snoring pattern is detected, a motorcycle sound is then detected, and an interruption of the snoring occurs two seconds after the motorcycle sound, then the relationship between the motorcycle sound and the snoring is able to be determined.

Video, motion and/or other information are able to be analyzed similarly. For example, video processing is able to be implemented to detect movement in a video and to classify the movement. Furthering the example, a user rolling over is able to be classified as rolling over or in a more generic category of a standard sleep motion, but very rapid, sudden movements of a user's appendages (as determined by the speed/distance per frame an appendage moves) may be classified as sleep tremors or a sleep disorder.

Analysis of the sleep information is able to include correlating the acquired information with other acquired information (or other information). For example, a break in a snoring pattern may correspond with movement, and the multiple aspects of information may provide more usable information for analysis.

Further sleep analysis is able to be performed. For example, sleep analysis includes analyzing different aspects of a user's sleep since sleep includes a variety of modes including light sleep, deep sleep, waking state, and/or other aspects of sleep. Therefore, a user's sleep state is able to be determined using the acquired information and/or other information. For example, based on the current time, and the sound pattern detected it may be determined that the user is currently in a light sleep. Furthering the example, based on detected information such as the ambient light level being below a threshold and the sounds of the user entering bed (e.g., creaking of the box spring), it is determined that the user went to bed at 10 p.m. At 10:15 p.m., the user's breathing pattern becomes consistent, so it is determined that the user is in a light sleep. Based on previous sleeping information, general sleeping information (e.g., based on medical studies) and currently acquired information, at 11:00 p.m. it is determined that the user is in a deep sleep. Determining how often a user enters each phase of sleep and how long the user is in each phase of sleep is able to be used to determine the quality of the user's sleep and provide further information such as recommendations, prohibitions and/or other information. Additionally, correlating external sounds with the sleep information is also able to provide further usable information. For example, if an external sound such as traffic wakes a user during the light sleep stage, the effect may be less when compared with a sound waking a user during the deep sleep stage. Thus, analyzing and learning based on the different information is able to provide a full analysis of the user's sleep.

Analyzing the acquired information is able to be used to monitor known sleep pathologies or detect unknown sleep pathologies such as excessive snoring, sleep apnea and various other conditions and problems. Machine learning is able to be used to detect sleep pathologies such as detecting a lack of a breath sound for a longer period than a normal threshold (e.g., apnea) or detecting snoring that reaches a decibel level above a threshold. By monitoring known problems, a user is able to determine if his condition is worsening. A user may be unaware that a sleep pathology is causing him problems, so detecting the pathology could be extremely helpful.

Analyzing environmental conditions or other external factors is able to provide useful additional information as to potential factors for better or poor sleep quality. Information such as temperature, background noises, light sources, and other factors are able to affect sleep quality. The information is able to be identified and categorized such as televisions playing during sleep, other people or animals present, noises (e.g., traffic noise), and activities noticeable to the sleeping user. The analysis involves tracking, classifying, and associating effects of the external factors and a user's sleep quality to improve the learning of the device. The learning is able to be shared generally but is also able to be person-specific. For example, if it is learned that a sound above 100 dB will awaken 99% of the users, then that information is useful when monitoring a user. Furthering the example, a sound of 102 dB was detected, and the user woke up 2 seconds later, those two events are able to be correlated, and a person having lower quality sleep that night can be explained. However, for the 1% of users who are deeper sleepers, a detection of a 102 dB sound, would not be a likely explanation for a poor quality of sleep.

Analyzing the acquired information is able to include determining which user/person is making which sounds and/or performing which actions. For example, if two people are sleeping, but only one is snoring, it is important to identify and attribute the snoring to the correct person. Identifying the correct person is able to be based on voice/sound analysis, video analysis and/or any other analysis. For example, a training period is able to be implemented where each user sleeps alone with the device for a set period of time for the device to learn user-specific details (e.g., User A's snore is much deeper than User B's snore; or User A snores, and User B does not). The device or devices are able to continuously learn or be used to learn as the device is used. Similarly, if a voice is detected, it is important to determine who is talking—is it the user talking in his sleep, is it the user's partner talking in her sleep, is someone on television, or is it a roommate talking while not sleeping? Voice detection is able to be performed via learning as the device continuously monitors and learns a user's voice as well as people around the user. For example, when a new voice is detected, the device is able to prompt the user to input an associated name with the voice or the device is able to assign the user a name.

Analysis of the acquired information is able to be performed to determine a sleep quality value. For example, based on the breathing information and other acquired information, it is able to be determined how many times the user enters each phase of sleep and how long each phase of sleep is, and if there are any events (e.g., interruptions) that occur. For example, based on sound analysis, it is determined that the user sleeps for eight hours, goes into and out of each sleep phase as typically occurs, and there are no interrupting events. A sleep score of 100 (from 1 to 100) may occur for such a night's sleep. However, if the device/system determines the user slept for 3 hours, never reached a deep sleep phase, and had the sleep interrupted by loud sounds 5 times, the sleep score may be 20 or another similarly low score. The sleep score is able to be used/analyzed in conjunction with analytics/challenge information as described herein.

In the step 5304, passive analytics and/or active challenges are implemented, and any correlations to the analyzed sleep information are determined. As described herein, the user device is able to be used to monitor and analyze user movement activities during non-sleep times. These activities include: movement performance such as walking, running, typing, reflex performances and other motion analytics which are able to provide energy and performance metrics for the user; overall amount of user movements and motions; impact of certain physical ailments on movement and sleep; an eating schedule; and more. The movement analytics are able to determine individual user energy and health levels when measured over time. Reduced movements or movement performance are able to be accurately measured. With these and other factors monitored and recorded, sleep quality is able to be correlated to daily energy levels and motion performance. In addition, breath pattern analysis and behavioral qualities such as movement analysis are also able to be used to determine the effect of people using the bedside user device, which is able to allow a deeper understanding of their moods and how certain events, or substances impact their performance.

In an example, a user slept poorly the previous night based on interruptions detected, the amount of deep sleep obtained and other factors. When the user types using the mobile device, it is detected that the user is much slower and makes more mistakes than usual (or the device detects the user is walking more slowly and/or is slurring his speech). Since the passive analytics are able to continuously occur, a user's entire day is able to be monitored by the device. For example, it is determined the user is less active and moves more slowly. The user's previous night or nights sleep are able to be analyzed as well, and a correlation between poor sleep and inactivity is able to be determined. In another example, based on the sleep information and/or passive analytics, an active challenge is triggered for the user to prove that his coordination and/or reflexes are normal such as following a set of directions to position the phone in certain ways and/or type specified words. If the device determines that the user slept poorly the night before, but there are no effects detected by the device during the day, then no further action may be taken (e.g., if the user is able to function well with little or poor sleep, then the device should not restrict the user's activities). In some embodiments, the correlation is able to be made over a longer period of time (e.g., 3 nights of little or poor sleep may ultimately lead to delayed reaction times, so the warning or restrictions come after the specific pattern/amount of time is detected). In some embodiments, the passive analytics and/or active challenges are based on detected/analyzed events during the user's sleep. For example, if there are zero unexpected awakening events, then no extra active challenges are implemented the next day. However, if there are 4 detected awakening events, then specific passive analytics and/or active challenges are implemented to determine if the user has been affected by poor sleep. The sleep score as described herein is able to be combined with a user's day score for a combined score. For example, if the user has a high sleep score by sleeping well, and is highly active, the user receives a high combined score (e.g., 100). In another example, the user has a low sleep score due to many sleep interruptions and a shortened night sleep, and the user has a less active day (e.g., based on learning and when compared with activity levels of other days by the user), the user receives a low combined score (e.g., 25). In a third example, the user has a low sleep score, but still has high activity levels, the user may still receive a high combined score (e.g., 90) depending on the implementation.

In the step 5306, a function is performed based on the correlations of the analyzed sleep information and the analytics/challenges. For example, if it is determined that the user's reflexes appear to be slower than usual based on a poor night of sleep (e.g., reflex time is lower than a threshold time), then an alert is triggered on the user's mobile device that he should be extra careful when driving or operating heavy machinery. In another example, a user is prohibited from driving (e.g., vehicle will not start after receiving a signal from the user's device or the vehicle goes into auto-pilot mode) due to the previous night's sleep information. In some embodiments, the function performed is based on a combination of the sleep quality information and the analytics/challenges results. For example, if the analyzed sleep information shows the user had a good night sleep, then the further analytics/challenges are not performed, and no restrictions are placed upon the user. In another example, if the analyzed sleep information shows the user had a poor night sleep, but the analytics/challenges do not indicate any reduced capacity/ability, then no restrictions are placed upon the user. In yet another example, if the user had a poor night sleep, and the user's reaction time is detected to have decreased, then alerts/restrictions occur. In some embodiments, the user's combined sleep score determines the functions performed. For example, if a user's combined sleep score is below a threshold (e.g., 30), then the user is prohibited from driving. In some embodiments, the function performed is not based on correlations of the analytics/challenges, but rather on detected events/quality of the sleep. For example, a report is generated each morning of the user's sleep. Furthering the example, in the report, it is stated that the user woke up 4 times, 3 of which are attributed to noises above 60 dB, and the other time is attributed to a cat jumping on the user. In some embodiments, fewer or additional steps are implemented. In some embodiments, the order of the steps is modified.

FIG. 54 illustrates a diagram of a bedside user device and system according to some embodiments. A user device (e.g., mobile phone) 5400, a wearable device (e.g., smart watch) 5402, sensors 5404, and/or security cameras 5406 are able to be used to acquire user information; particularly, user information while the user sleeps. The user device 5400 is able to be positioned near the user's bed, on the user's bed or elsewhere. Fewer or additional types of devices are able to be included in the system. For example, a system is able to be without a security camera 5406, or instead of a security camera, a camera on a television is used for video monitoring. The devices are able to communicate with each other and/or a network device 5408. The processing/analysis is able to be performed locally (e.g., on the user device 5400) or remotely (e.g., on the network device 5408). Via synchronized processing, the devices are able to be used in combination for refined analysis. For example, if the user device 5400 detects the sound of sheets moving, and the security camera 5406 detects the user moving with the same timestamp, then the system is able to be more confident that the sound detected is the sheets moving based on the user moving. Each of the devices is able to have one or more receiving/transmitting components. For example, the user device 5400 is able to include a microphone for acquiring sound, a camera device for acquiring images/videos, and one or more sensors for detecting movement.

The user device 5400 is able to be carried and/or used as described herein to perform additional passive analytics and/or active challenges and to provide further analysis/results of the sleep analysis. The other devices are able to be utilized to perform passive analytics and/or active challenges.

In some embodiments, the user information acquired while the user is sleeping is able to affect the trust score of the user of the device. For example, a user's breathing pattern is able to be stored on the device to then be later used for real-time comparison of the user's current breathing pattern to keep the trust score of the device at a level higher than if the user puts his phone down. Furthering the example, if the user puts his phone down (e.g., during the day), the trust score drops to a specified amount (e.g., 50) or below a threshold. However, if the user puts his phone down to sleep, but the phone detects the user's breathing pattern (or other information while sleeping such as snoring volume or pattern, motion pattern and others) and a match is found based on previous breathing pattern/sleep information, then the trust score may be increased from 50 (for putting the phone down) to 70 or another set amount such that the device is more confident that the person who picks up the device is the user (e.g., in the morning). In some embodiments, further analysis is performed such that if the device is still receiving breathing or other information indicating that the user is still sleeping, and the device is picked up, then the trust score on the device drops to the standard pick up trust score (e.g., 50), a lower trust score or even locks the device, as it is highly likely that the person who picked up the device is not the user. Further behavioral analytics are able to be utilized such as the user's sleeping habits to determine the trust score. For example, if the user typically goes to sleep at 10 pm and regularly wakes up at 6 am without touching the phone, then the trust score of a person picking up the device at 6:05 am is able to be at an amount consistent with trusting that the person is the authorized user. However, if a person picks up the device at 1:00 am, the trust score would be a lower amount (e.g., 30) since this is not a consistent behavior. As described, since each individual has different behaviors, the trust scores are able to be affected differently depending on past behavior. If a different person typically wakes up 3 times at night and reads on his device each time he wakes up, then the trust score may not be lower (e.g., 70) at those odd hours, since it actually matches with the user's prior behavior.

Health and mood monitoring is able to be performed using passive analytics and/or active challenges by correlating responses with environmental factors and how they affect conditions in a positive or negative way. For example, how do employees (or their performance) react to different environments.

A user device is able to be used to monitor human external conditions such as: medications, therapies, diets, and others. The device is also able to provide health condition feedback as the device correlates effects with the external conditions. The device is able to provide plans and monitor the user (e.g., behaviors) while the plans are executed. The device is also able to provide feedback to the user (e.g., suggestions for improvements to the user's performance). The device is able to continuously gather data including various input and behavioral output which are then able to be analyzed via machine learning to determine if any correlations or patterns are determined. The acquired data and analysis are able to be shared (e.g., to a network device) for further analysis. For example, 1 billion people across the world share the information acquired by the device, and a central device (e.g., one or more supercomputers) processes the data by grouping/categorizing the data and determining any correspondences in the data. For example, one person drinking a diet drink for a month and gaining 5 pounds is not particularly useful in providing a usable correlation; however, if 1 million people drink the same diet drink for a month and gain 5 pounds on average, then this correlation may be useful and is able to be reported. Since a large amount of data is acquired for each user, additional correlations may be found as well which may refine a correlation or correct an errant correlation. For example, if it is also determined that the 1 million people who drink the same diet drink also eat on average 10,000 calories per day and do not exercise, then the correlation between the diet drink and the weight gain is not as strong or may even be incorrect, and the correct correlation is the calories and/or lack of exercise. The device is able to detect when someone is walking slower than usual, breathing differently, has more accidents, performs poorly on tests, and/or any other behavioral performance changes.

Factors that are able to be analyzed to determine correlations include: voice tone, quality and speed, bodily movement analysis, breath analysis, device usage, grip strength, body temperature, speech analysis, text/typing analysis (e.g., detecting words such as “ouch,” “pain”), energy levels, quality/performance of the factors, physical exercise, mental exercise, physical/mental therapy (e.g., meditation), external factors (e.g., diet, medication, weather, noise, stressful versus serene environments), and others.

A user's mood is able to be determined based on the factors.

The health and mood monitoring is able to be implemented with an immediate aspect and a long-term aspect. The immediate aspect is able to perform analysis of user information for real-time results including making immediate recommendations such as alerting a user to not drive or preventing a user from driving. The long-term aspect is able to perform the analysis over a longer period of time (e.g., days, weeks, months, years) and involves collecting large amounts of data from multiple inputs and multiple sources; determines correlations between the input; and potentially provides feedback to the users (e.g., adjust eating behaviors, exercise more).

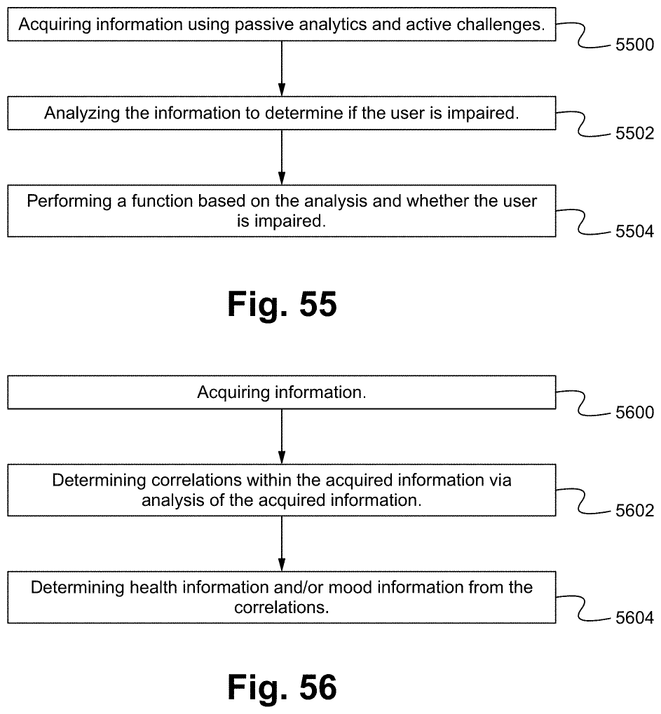

FIG. 55 illustrates a flowchart of a method of implementing an immediate health and mood monitoring system according to some embodiments. In the step 5500, information is acquired. The information is able to be user information and/or external information.

The user information is able to include user attributes such as voice tone, voice quality and voice speed, bodily movement analysis, breath analysis, device usage, grip strength, body temperature, speech information, text/typing information (e.g., detecting words such as “ouch,” “pain”), energy levels, quality/performance of the factors, physical exercise, mental exercise, physical/mental therapy (e.g., meditation), facial/body expressions, and others. The user information is able to be acquired as or from audio, video, image, sensor, and/or other information. The user information is able to be acquired using the device to implement the passive analytics and/or active challenges described herein.

The external information is able to include external factors such as diet, medication, weather, noise, stressful versus serene environments, and others. The external information is able to be acquired in any manner such as by a user manually inputting in information (e.g., typing or selecting the meal the user ate), retrieving information from a receipt (e.g., shopping receipt indicates which items a user purchased or a restaurant receipt includes the meal a user ate), extracting information from a picture (e.g., a user takes a picture of the user's meal, and the device is able to analyze the picture to parse out each item and calculate dietary information such as calories, protein, vitamins, minerals and more), and in other ways. The external information is able to be acquired by: sensors (e.g., a thermometer), searching for and acquiring information (e.g., search via a search engine to retrieve data), a microphone/camera, and/or any other manner.

The passive analytics and/or external information are also able to be used to determine that a user is currently participating in an activity that may result in an impaired state at a later time. For example, a wearable device is able to acquire arm movement information that indicates repeated drinking motions. External information such as GPS information that the user's current location is at a bar would further suggest the possibility of the user drinking alcohol. This information is able to be coupled with information acquired at a later date such as modified user movement information to provide a more accurate analysis/determination.

Various devices are able to be used to acquire the user information and/or the external information. For example, a mobile phone, a wearable device (e.g., smart watch), a stationary device, and/or other devices are able to acquire the user information and/or the external information, as described herein.

In the step 5502, the acquired information is analyzed. As described herein, sound processing, image/video processing, sensor processing (e.g., motion analysis), and/or other processing is able to be implemented. Machine learning is also able to be implemented to perform the analysis. For example, pattern matching is able to be implemented by repeatedly processing information and learning to detect patterns.

As described herein, training/stored information is able to be classified and then used in the analysis of the current user information. For example, during a training period, a user indicates that he is intoxicated/under the influence of a foreign substance, and the motion information acquired during that period is able to be used for pattern detection/matching to determine if a user is currently intoxicated. The stored information is able to be acquired at any time or continuously acquired. For example, if the user is using the device regularly, and then the device detects that the user's movements are different from usual, the device is able to query the user if he is intoxicated. If the user responds in the affirmative, then that acquired data is able to be stored and used for later pattern matching. In some embodiments, the device performs the classification automatically without a user response. For example, the based on previously stored information and/or learned information from other users, an intoxicated user typically has a gait that is 10%-25% slower than the user's average gait, and the gait is not in a straight line, so when the device detects a user's gait that matches this learned pattern, the device determines that the user is likely intoxicated.

In some embodiments, a device attempts to find a match with the currently acquired user information from the analytics and/or challenges with a modified version of the stored information, or the stored information with a filter/modifier applied to it. For example, if a user's average acceleration and/or velocity information for a shake challenge is X, then to check for or determine that the user is intoxicated, the analysis compares the currently acquired acceleration/velocity information is within the range of X*0.75 through X*0.9 (e.g., 10% -25% slower). The modifier is able to be determined in any manner such as by implementing learning with a large number of users to determine the modifier. Similarly, the angle information of the shake or ability to move the device in a consistent direction is able to be analyzed and determined.

As described, there are at least two ways to determine if the user is impaired in some way or has a condition. User information is able to be acquired, classified, and stored when the user's abilities are impaired. For example, when a user is very tired and has slower reflexes, the user's gait, shake challenge information, repeated yawns, drooping eyes, and/or any other effects are able to be acquired and stored in separate classifications and possibly sub-classifications. Then, newly acquired user information is able to be directly compared with the information in the separate classifications for a match (e.g., user's current gait matches with the user's gait when intoxicated instead of when the user is not intoxicated). When user information has not been stored in classifications yet or in some embodiments, the currently acquired user information is able to be compared with modified/filtered information for comparison purposes to determine if the user is not functioning in a typical manner.

The user information is able to include a health score/rating. For example, the health score is able to be from 0-100, where 0 means asleep or severely impaired and 100 means fully awake and not impaired at all. A user is able to start at a baseline of 100 or another value, and then based on the passive analytics and active challenges, the user's health score is able to increase or decrease. For example, if a user gets a poor night sleep, the user's health score in the morning may be 50, but after the user exercises the health score is up to 60, but then at night after the user has had 2 drinks, the health score drops to 25. If a threshold for the user to be able to drive (or perform some other activity) is 30, then an action is able to be taken to prevent the user from driving. In some embodiments, a user is able to have multiple scores which are able to be analyzed separately and/or affect an overall score. For example, a user may have a reflex score which is affected by sleep quality, substances that affect reflexes, and/or other activities, and the user may have a health score which is affected by the user ailments, exercise and food/drinks ingested. Different activities affect the scores differently (although some activities may affect both scores). The scores are able to be compared separately or in combination with one or more thresholds for providing alerts and/or taking actions.

A distinction is able to be made from when a different user is using the device and when the authorized user is using the device but is impaired. For example, passive analytics and/or an active challenge that are not affected by sleep/alcohol/other impairments are able to be used to confirm the user's identity, and passive analytics and/or an active challenge that are affected by sleep/alcohol/other impairments are able to be used to confirm that the user is impaired.

A user's mood is able to be determined by facial expressions, body expressions, voice tones, and other information which are able to be analyzed. The various information is able to be classified and compared to stored classified information. For example, a smile is classified or correlated with a happy mood; whereas, a frown or tears are classified with a sad mood. The user information is able to be continuously acquired and analyzed which is able to continuously affect the user's mood. In some embodiments, the user has one or more mood scores/ratings. An overall mood rating is able to be a grouping of separate mood ratings. For example, one aspect of a mood rating is happy versus sad, where 100 indicates very happy, and 0 indicates very sad. A second aspect of a mood rating is calm versus anxious where 100 indicates very calm, and 0 indicates very anxious. Since there are many opposite moods/emotions, it may make sense to classify them separately. For example, a user who is very sad but is also very calm would receive a combined score of 50, while a user who is very happy but is also very anxious would also receive a score of 50. Therefore, analysis of each mood separately may be more effective or at least in a more complex way than combining the numbers. The mood information and/or score are also able to be used to provide alerts/recommendations and/or take actions. The mood information and/or score are able to be analyzed in combination with the other acquired information. For example, an alert may be triggered for an angry user who is intoxicated, whereas a happy, intoxicated may trigger a different alert or no alert (except if a vehicle is involved).

External information is able to be analyzed in any manner depending on the external information. The external information is able to be correlated with any of the other acquired information. For example, GPS location information and temperature information are able to be correlated with a user's motion information and/or sensor information. Furthering the example, if a user's mobile device detects excessive perspiration, this could be a sign of a medical condition or it could be based on an activity or the temperature. By correlating the current temperature of 85 degrees and GPS information which indicates that the user is running, it is able to be determined that the user's perspiration is based on user activity, not a medical condition.

The analysis is able to be performed on a user device and/or a network device. The analysis is able to use multiple sources of input for machine learning. For example, by analyzing many users' information, general patterns are able to be learned, and the general patterns are able to be refined using the user's specific information. For example, by analyzing many users' movement information, general trends/patterns are able to be determined for when a user is intoxicated such as a user's arm movement of lifting a glass to his mouth a repeated number of times, a user's gait being slower and less stable (e.g., more side-to-side movement), a user's breathing and heartrate slowing, delays in eye movement reaction time, slower/slurred speech and others. The learning information is able to be refined by analyzing the specific user information. For example, the specific user's heart rate is already slow, and alcohol does not affect his heart rate, but the other effects are felt by the user, so different aspects may be included/excluded when searching/matching for analysis. Also, general numbers are able to be determined based on the overall information and then narrowed for a specific user. For example, in general, a users' gaits slow by a range of 10%-25% when they are intoxicated. However, User A's gait slows by 20%-25% consistently when he is intoxicated, so that narrower range is able to be used when analyzing that user's information, which will help avoid mischaracterizing a user's actions.

In the step 5504, a function is performed based on the analysis. The function or action performed is able to include sending an alert, a prohibition of an activity, and/or any other function/action. For example, if it is determined that the user's reflexes appear to be slower than usual, then an alert is triggered on the user's mobile device that he should be extra careful when driving or operating heavy machinery. In another example, a user is prohibited from driving (e.g., vehicle will not start after receiving a signal from the user's device or the vehicle goes into auto-pilot mode) due to the analysis of the user information. If a medical emergency is detected, a 911 call is able to be triggered. In some embodiments, fewer or additional steps are implemented. In some embodiments, the order of the steps is modified.

Any of the implementations described herein are able to be used with any of the other implementations described herein. In some embodiments, the implementations described herein are implemented on a single device (e.g., user device, server, cloud device, backend device) and in some embodiments, the implementations are distributed across multiple devices, or a combination thereof.

FIG. 56 illustrates a flowchart of a method of implementing a long-term health and mood monitoring system according to some embodiments. In the step 5600, information is acquired. The information is able to be user information and/or external information.

The user information is able to include user attributes such as voice tone, voice quality and voice speed, bodily movement analysis, breath analysis, device usage, grip strength, body temperature, speech information, text/typing information (e.g., detecting words such as “ouch,” “pain”), energy levels, quality/performance of the factors, physical exercise, mental exercise, physical/mental therapy (e.g., meditation), facial/body expressions, and others. The user information is able to be acquired as or from audio, video, image, sensor, and/or other information. The user information is able to be acquired using the device to implement the passive analytics and/or active challenges described herein.

The external information is able to include external factors such as diet, medication, weather, noise, stressful versus serene environments, light information, and others. The external information is able to be acquired in any manner such as by a user manually inputting in information (e.g., typing or selecting the meal the user ate), retrieving information from a receipt (e.g., shopping receipt indicates which items a user purchased or a restaurant receipt includes the meal a user ate), extracting information from a picture (e.g., a user takes a picture of the user's meal, and the device is able to analyze the picture to parse out each item and calculate dietary information such as calories, protein, vitamins, minerals and more), and in other ways. The external information is able to be acquired by: sensors (e.g., a thermometer), searching for and acquiring information (e.g., search via a search engine to retrieve data), a microphone/camera, and/or any other manner.

Various devices are able to be used to acquire the user information and/or the external information. For example, a mobile phone, a wearable device (e.g., smart watch), a stationary device, and/or other devices are able to acquire the user information and/or the external information, as described herein.

The information is able to be acquired over long periods of time such as days, weeks, months or years. The information is able to be acquired for a single user and/or many users. For example, a social network of users' information is able to be acquired.

In the step 5602, correlations are determined within the acquired information via analysis of the acquired information.

As described herein, sound processing, image/video processing, sensor processing (e.g., motion analysis), and/or other processing is able to be implemented. The analysis is able to be used to separate information into specific pieces of information to be compared and/or classified. For example, if a device captures information while a user is running, the information captured is able to include GPS information, time information, breath information, grunting noises, gait, arm motions, perspiration information, body temperature, heart rate, and many other separate pieces of information which are able to be classified. Machine learning is also able to be implemented to perform the analysis. For example, pattern matching is able to be implemented by repeatedly processing information and learning to detect patterns.

As information is acquired, the information is stored to further machine learning about the user. For example, information is able to be classified in categories or sub-categories based on the analysis. Furthering the example, food/diet information is able to be acquired and stored in a food category and/or a sub-category depending on the type of food (e.g., healthy, junk), based on the time of day (e.g., breakfast, lunch, dinner) or any other distinction/classification.

Acquired information is able to be classified as a cause or an effect; or symptom or cure; trigger or mood; or other binary classification. Other opposing categories are able to be implemented, or multiple categories are able to be implemented (e.g., trigger, symptom, cure). In some instances, the same information is able to be classified in multiple categories. The acquired information is able to be classified in a temporary classification and then moved into a more permanent classification depending on further analysis. For example, a food is able to be classified as a potential allergen (e.g., cause), and then based on further analysis, it is determined that the food is not causing the symptoms, so the food is removed from the “cause” classification. Acquired information is able to be unclassified initially and then classified as a cause or an effect after further analysis.

Analyzing the information is able to include determining if there are any relationships (e.g., cause and effect) that repeatedly occur. For example, if it is detected that every time a user drinks a cup of coffee, the user's hands have excessive microtremors, then a correlation has been determined. For health, a timing relationship is able to be analyzed. Furthering the example, some effects occur within a specific amount of time such as an allergic reaction to food or another digestive/physical reaction to the food. Therefore, when searching for some correlations (e.g., ones that have a timing factor), the analysis is able to reduce the amount of information to be compared. For example, in trying to determine a correlation with a user's repeated stomach aches, the analysis is limited to foods that the user ate within the last 72 hours before each stomach ache started instead of all of the foods that the user ate instead of all of the food that the user ate over his lifetime. Other optimizations are able to be implemented when analyzing the information to determine correlations. Additionally, optimizations are able to be learned. Moreover, some optimizations are able to apply to certain information for one type of correlation but not for another type of correlation. For example, for relatively immediate effects such as a stomach ache, limited information (e.g., 3 days of food consumed by the user) is able to be used, but for long-term effects such as weight gain, a much larger body of information is able to used (e.g., food consumed by the user over his lifetime along with other information).

Analyzing the information is able to include determining if any common effects, symptoms, illnesses, and/or others occur. For example, if a user or many users have a symptom in common (e.g., headache or breathing difficulties), then causes/triggers of that symptom are able to be searched for to determine if there is a number of matches above a threshold. Additionally, cures of that symptom are able to be searched for. Searching for aspects (e.g., causes) in common is able to be implemented in any manner such as using machine learning and/or pattern matching to locate matching instances/aspects, and determining if the number of matches is enough to indicate a pattern or correlation.

In some embodiments, effects are searched for, and then causes are searched for based on the effects. For example, a symptom, an illness, and/or any other effect is able to be searched for. The search is able to be limited to a specific user or to many users. For example, a headache symptom is searched for in a system's entire community (e.g., millions of users). Once the instances of headache symptoms are found, then the acquired information for each user is searched that predates each instance of headache. The search is able to be narrowed (e.g., by time proximity), although a second path of a non-narrowed search is also able to be implemented in parallel or afterward. For example, a headache is typically based on events that occurred within the past 24 to 72 hours such as loud noises, not sleeping well, not drinking enough water, stress, bright lights, physical contact with the user's head, and/or other typical causes. Therefore, a search of the user information for that time period for matches is able to occur. Additionally, some user headaches are able to be based on long-term issues such as a tumor, hormones, and/or another cause. The second path of searching is able to include searching for matches/patterns over a longer period of time such as frequent cellular phone use which could lead to a brain tumor.

The search/analysis of the predating information attempts to find causes in common. A cause in common or any aspect in common is able to be based on a number or a percentage of causes/aspects when compared with a threshold. For example, if a headache is found in 100 users, and in 10 of those users, a cause in common is drinking alcohol, then the alcohol is a cause in common if the threshold is 10% or a lower threshold. In some embodiments, multiple thresholds are able to be implemented to further classify causes/aspects. For example, a cause that occurs in less than 2% of the time related to an effect is considered rare, a cause that occurs in 2% to 10% is considered uncommon, and a cause that occurs above 10% is considered common. In another example, if many of the users had headaches in the morning, and it is determined that they performed a lifting of arm to mouth motion (e.g., drinking something) and also had slower movements combined with poor balance based on passive analytics, it is able to be determined that those headaches were likely caused by drinking too much alcohol. However, another set of users had headaches the following day after viewing a screen (e.g., TV, computer) in a dark setting the night before. This grouping of information is able to be used to determine the cause of those users' headaches was likely the blue light from the screens.

In some embodiments, if multiple causes are determined to be correlated or associated with one or more effects, further analysis is able to be performed to determine if one of the causes is a more major/dominant cause and/or if any causes are incidental. For example, if multiple correlations are found in one set of data (e.g., a single person), then a larger set of data is analyzed which provides a clarification of causes (e.g., one cause was not found as often or was found fewer times than a threshold in the larger set of data, so it is able to be removed as a cause for the user).

In some embodiments, multiple symptoms are detected and analyzed to distinguish between causes that have overlapping symptoms since some illnesses have similar symptoms with some distinctions. For example, Covid-19 has symptoms similar to a cold, but if the user loses taste/smell as one of the first symptoms, it is likely that the user has Covid-19 and not a cold. Therefore, the symptoms in common are able to be used to narrow the possible cause down to multiple causes, and then any distinctive symptoms are able to narrow the possible cause down even further (possibly to one cause).

In an example of mood analysis, user moods are able to be searched for, and then a cause in common for the user mood is able to be determined. For example, a sad user mood is able to be detected based on sound information of a user crying, image information with a user frowning and/or with tears, and/or text information where a user types that he is “sad” or a synonym. Analysis of that user information and/or other user information preceding the detection of the sad mood is able to occur. For example, the device searches for causes of the sad mood. Causes are able to include an injury/health issue, family/relationship/pet issues, work-related issues, a crime, and many others. In some embodiments, the analysis/matching is also able to include matching what has previously been learned. For example, if the current user is sad based on a detected health issue, the device is able to compare the user information with stored known issues which cause sadness and determine that the health issue is likely causing the user's sad mood.

In the step 5604, health information and mood information are able to be determined based on the correlations/analysis. Based on the cause and effect or other analysis, a diagnosis and treatment are able to be determined and suggested. For example, if a headache is detected in a user, the suggested treatment for the user may depend highly on the cause of the headache. If the headache is based on drinking too much alcohol, then a suggestion may include drinking extra water and resting. If the headache is based on a physical injury, then Tylenol and an icepack may be suggested. If the headache is determined to be a repeated symptom with no clear immediate cause, then a suggestion to see the doctor and have medical imaging performed may occur. For mood analysis, suggestions on improving and/or changing a user's mood may be implemented. For example, if a user is sad due to being lonely, then images of the user's family, friends and/or pet are able to be provided to cheer up the user, or a phone call to a family member may be recommended. In some embodiments, fewer or additional steps are implemented. In some embodiments, the order of the steps is modified.

The analysis is able to be performed on a user device and/or a network device. The analysis is able to use multiple sources of input for machine learning. For example, by analyzing many users' information, general patterns are able to be learned, and the general patterns are able to be refined using a user's specific information. For example, by analyzing many users' movement information, general trends/patterns are able to be determined. The learning information is able to be refined by analyzing the specific user information. Also, general numbers are able to be determined based on the overall information and then narrowed for a specific user.

Clinical drug trial data is able to be enriched using activity and behavioral analytics captured with personal devices and applications. Users are able to participate in medical trials (e.g., drug, herbal, vaccine trials), and the behavioral analytics and/or other activity information of the user are able to be used to determine medical aspects related to the medical trials such as effectiveness, side effects and/or other aspects of the medication.

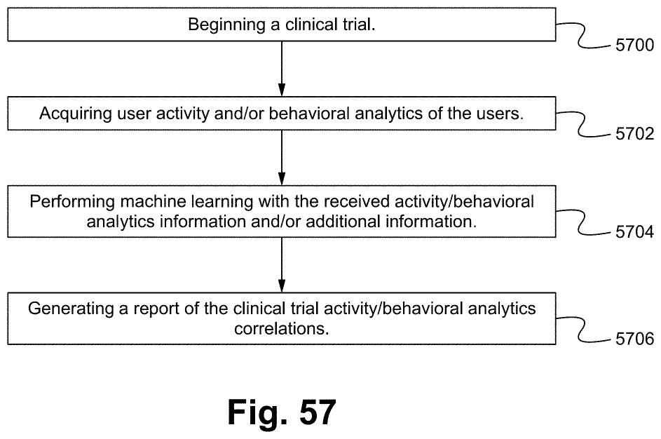

FIG. 57 illustrates a flowchart of a method of utilizing activity and behavioral analytics to enrich clinical trial data according to some embodiments. In the step 5700, a clinical trial begins. The clinical trial is able to be a drug trial, a vaccine trial, a biological product trial or any other trial. The clinical trial involves participants receiving specific interventions according to a research plan or protocol developed by investigators. The interventions are able to be medical products, such as drugs or devices; procedures; or changes to participants' behavior, such as diet. Clinical trials are able to compare a new medical approach to a standard one that is already available, to a placebo that contains no active ingredients, or to no intervention. Some clinical trials compare interventions that are already available to each other (e.g., medication A vs. medication B). When a new product or approach is being studied, it is not usually known whether it will be helpful, harmful, or no different than available alternatives (including no intervention). The investigators try to determine the safety and efficacy of the intervention by measuring certain outcomes in the participants.

In the step 5702, while the clinical trial is ongoing, devices acquire user activity and/or behavioral analytics of the users. For example, each user's mobile phone includes an application which is able to perform the behavioral analytics as described herein. Furthering the example, behavioral data such as habits, diets, exercise, sleep patterns, and smoking/drug use is able to be acquired. In addition to a user's mobile phone, other devices are able to be used to capture activity and behavioral information of the user such as a motion system embedded in a user's bed, a wall mounted device, a wearable device, an exercise machine, and/or any other device.

The devices are able to acquire, analyze, and/or transmit the activity/behavioral information. For example, a mobile device is able to acquire a user's gait and send the gait information to a server device for analysis. In another example, the mobile device performs the analysis and then sends the analyzed information to the server. As described herein, in addition to acquiring the activity/behavioral information, related information is able to be acquired, analyzed and/or transmitted such as condition information, environmental information, mood/medical information, and/or other information.

The information acquired is able to include a wide variety of information including specific details of movements/behaviors by the user, medical information (e.g., vitals, medication taken), date/timestamps, temperature, lighting, weather, GPS, and/or any of the other information described herein.

In the step 5704, one or more server devices perform machine learning with the received activity/behavioral analytics information and/or additional information. The machine learning is able to include updating learned information about each user, detecting matches and/or trends, and/or performing other comparisons with the acquired information. The server devices utilize pattern matching and/or other machine learning analysis to determine if any trends or other commonalities are detected. For example, if a clinical trial includes 1000 participants for a new blood pressure medication, and the behavioral analytics information indicates that a side effect of the medication is that the users sleep an average of 2 hours more than before taking the medication, the server devices are able to detect the delta of sleep before the clinical trial started and during the clinical trial. By utilizing an application that performed the behavioral analytics before the clinical trial began, a device is able to have knowledge of the users' activities before the clinical trial began which is able to be used to find deltas of behavior. Instead of a user having to answer a questionnaire or note side effects, the machine learning is able to automatically detect the side effects. Moreover, the machine learning is able to detect correlations that are undetected by the user. For example, a user may not notice that his reflexes are delayed for several hours after taking the blood pressure medication, but the behavioral information is able to be analyzed and the delayed reflexes are able to be detected. Furthermore, the information is able to be more accurate than when a user provides responses to a questionnaire. Users are able to provide false information, but if the user's behavior and other information are acquired automatically, it is much less likely that the user is able to fake, for example, slurred speech or slower reflexes for an extended period of time.

In another example, a sleep aid being studied is provided to a group of participants, and some participants' activity information indicates they had more energy while taking the sleep aid, and other participants' activity information indicated no change in energy. Further analysis correlates that the participants who also exercise daily had the increased energy from the sleep aid, and participants who did not exercise often, received no benefit. The analyzed information is able to be used in conjunction with or instead of a participant questionnaire or any other participant reporting implementation. For example, if a user reports that she has increased energy on days after taking a sleep aid, the analyzed information is able to confirm or reject that user report based on how active the user actually was. Furthering the example, the device/application is able to detect the number of steps the user takes, record the pace of a run or other exercise, and/or any other analysis to determine how active a user is.

The activity/behavior analytics are able to be correlated with the study information. For example, behavior analytics detect a change in microtremors in 50% of participants hands approximately 30 to 50 minutes after taking medication X. Furthering the example, behavior analytics detect no change in microtremors in 100% of the participants taking a placebo. A side effect of microtremors may be clearly established by the behavior analytics. A significant benefit of the behavioral analytics is that the participants are not needed to be actively involved and are generally not able to fake or trick the system to provide false input. Moreover, performing a multitude of analytics, further refinement of data analysis is possible. Continuing the example, it is determined that 50% of the participants took their medication with coffee, which may cause microtremors. But then even further analysis includes previous behavioral analytics before the clinical study began, and 25% of the participants who took their medication with coffee did not previously have microtremors after drinking coffee. Then, further analysis is able to be implemented to determine if the combination of the medication and coffee is an issue or if there are other factors involved. By acquiring and analyzing massive amounts of behavioral, environmental, conditional and other data, from a large population size (e.g., 100, 1000, 1 million people, depending on the implementation), various correlations are able to be determined with significant precision and accuracy.

The acquired information is able to be grouped/classified in any manner. For example, behavior information is able to be classified as a symptom or a cause. As described herein, the information is able to be classified in any manner such as by comparing it with previously analyzed information (e.g., learning) or via relational analysis. For example, based on previous analysis, microtremors within 5 minutes of ingesting a Class A medication occur in 50% of people, and the current medication is a Class A medication and was taken 4 minutes ago, thus the microtremors are likely a symptom/side-effect from the medication. The information is able to be re-classified if further learning determines that another classification is more appropriate. In some embodiments, there are multiple sub-classifications to provide a very fine-tuned analysis. The sub-classifications are able to be structured in any manner such as hierarchical tree, a table, a chart or any other structure. Different aspects/information are able to linked. For example, a timestamp is able to be linked with a behavior which is able to be linked with a medication. Moreover, information from separate users is able to be grouped and/or linked.

The information is able to be combined, averaged, distinguished and compared with one or more thresholds. For example, the time for an effect to occur after a medication is taken may vary among the participants, but a range and/or an average are able to be determined. Deltas or other differences between acquired/analyzed information are able to be determined. The raw, averaged or delta information is able to be compared with one or more thresholds for analysis. For example, if a blood pressure medication is being studied, and 25% of the participants have an average heart rate increase of 2%, that information may not be relevant enough for it to be classified as a side effect since the increase is below a threshold (e.g., 10%). The thresholds are able to be used for classification and/or other aspects of machine learning.

Based on detecting/determining one or more correlations in a number of people above a threshold, a causation is able to be established. For example, there may be a correlation between a medication and weight gain, but with further analysis, it is determined that many people, even people who regularly exercise and eat well, experienced weight gain while taking the medication. Therefore, it is able to be determined that the medication is the cause or likely cause with a confidence level of the weight gain. The confidence level is able to be affected by the number or percentage of people who fit certain criteria for establishing the correlation.

In the step 5706, the server devices generate a report of the clinical trial activity/behavioral analytics correlations and/or causations. The report is able to be generated in any manner and include any relevant information such as indicating what percentage of participants have which effects and/or any cross-correlation of other aspects (e.g., secondary medications). The report is able to be very detailed and include all of the related information (e.g., timestamps, effects, identification information, conditional information, and more); the report is able to be general and simply provide side effect information and percentages; or the report is able to be somewhere in between.

In some embodiments, fewer or additional steps are implemented. In some embodiments, the order of the steps is modified.

The present invention has been described in terms of specific embodiments incorporating details to facilitate the understanding of principles of construction and operation of the invention. Such reference herein to specific embodiments and details thereof is not intended to limit the scope of the claims appended hereto. It will be readily apparent to one skilled in the art that other various modifications may be made in the embodiment chosen for illustration without departing from the spirit and scope of the invention as defined by the claims.

What is claimed is:

1. A method programmed in a non-transitory memory of a device comprising:
acquiring user information including medical information and behavioral information from a plurality of participants during a clinical trial;
analyzing the user information to generate behavioral analytics;
determining one or more correlations within the behavioral analytics and the medical information; and
establishing one or more causations based on the one or more correlations determined in a threshold of participants.

2. The method of claim 1 further comprising acquiring the user information before the clinical trial starts.

3. The method of claim 2 further comprising comparing the user information acquired before the clinical trial with the user information during the clinical trial to determine one or more deltas between the user information.

4. The method of claim 1 wherein the clinical trial comprises a clinical drug trial, a vaccine trial, or a biological product trial.

5. The method of claim 1 wherein the user information is acquired by a mobile device, a wall-mounted device, an embedded device, a wearable device and/or an exercise machine.

6. The method of claim 1 further comprising acquiring and analyzing condition information, situational information, and/or environmental information.

7. The method of claim 1 wherein determining the one or more correlations within the behavioral analytics and the medical information includes using machine learning to detect one or more patterns.

8. The method of claim 1 further comprising generating a report based on the one or more correlations.

9. The method of claim 1 wherein the behavioral information includes at least one of habits, diet, exercise, sleep patterns, and smoking.

10. A device comprising:
a non-transitory memory for storing an application, the application configured for:
acquiring user information including medical information and behavioral information from a plurality of participants during a clinical trial;
analyzing the user information to generate behavioral analytics;
determining one or more correlations within the behavioral analytics and the medical information; and
establishing one or more causations based on the one or more correlations determined in a threshold of participants; and
a processor configured for processing the application.

11. The device of claim 10 wherein the application is further configured for acquiring the user information before the clinical trial starts.

12. The device of claim 11 wherein the application is further configured for comparing the user information acquired before the clinical trial with the user information during the clinical trial to determine one or more deltas between the user information.

13. The device of claim 10 wherein the clinical trial comprises a clinical drug trial, a vaccine trial, or a biological product trial.

14. The device of claim 10 wherein the user information is acquired by a mobile device, a wall-mounted device, an embedded device, a wearable device and/or an exercise machine.

15. The device of claim 10 wherein the application is further configured for acquiring and analyzing condition information, situational information, and/or environmental information.

16. The device of claim 10 wherein determining the one or more correlations within the behavioral analytics and the medical information includes using machine learning to detect one or more patterns.

17. The device of claim 10 wherein the application is further configured for generating a report based on the one or more correlations.

18. The device of claim 10 wherein the behavioral information includes at least one of habits, diet, exercise, sleep patterns, and smoking.

19. A system comprising:
a first device configured for:
analyzing user information to generate behavioral analytics; and
determining one or more correlations within the behavioral analytics and medical information;
establishing one or more causations based on the one or more correlations determined in a threshold of participants; and
a second device configured for:
acquiring user information including medical information and behavioral information from a plurality of participants during a clinical trial.

20. The system of claim 19 wherein the second device is further configured for acquiring the user information before the clinical trial starts.

21. The system of claim 20 wherein the first device is further configured for comparing the user information acquired before the clinical trial with the user information during the clinical trial to determine one or more deltas between the user information.

22. The system of claim 19 wherein the clinical trial comprises a clinical drug trial, a vaccine trial, or a biological product trial.

23. The system of claim 19 wherein the second device comprises a mobile device, a wall-mounted device, an embedded device, a wearable device and/or an exercise machine.

24. The system of claim 19 wherein the first device is further configured for receiving and analyzing condition information, situational information, and/or environmental information.

25. The system of claim 19 wherein determining the one or more correlations within the behavioral analytics and the medical information includes using machine learning to detect one or more patterns.

26. The system of claim 19 wherein the first device is further configured for generating a report based on the one or more correlations.

27. The system of claim 19 wherein the behavioral information includes at least one of habits, diet, exercise, sleep patterns, and smoking.
